SEQUENCE LISTING (10 sequences)

SEQ ID NO: 1 - DNA, 1782 bp, Homo sapiens
tttggccctcgaggccaagaattcggcacgagggggggaggtgcgagcgtggacctgggacgggtctgggcggctctcggtggttggcacgggttcgcacacccattcaagcggcaggacgcacttgtcttagcagttctcgctgaccgcgctagctgcggcttctacgctccggcactctgagttcatcagcaaacgccctggcgtctgtcctcaccatgcctagcctttgggaccgcttctcgtcgtcgtccacctcctcttcgccctcgtccttgccccgaactcccaccccagatcggccgccgcgctcagcctgggggtcggcgacccgggaggaggggtttgaccgctccacgagcctggagagctcggactgcgagtccctggacagcagcaacagtggcttcgggccggaggaagacacggcttacctggatggggtgtcgttgcccgacttcgagctgctcagtgaccctgaggatgaacacttgtgtgccaacctgatgcagctgctgcaggagagcctggcccaggcgcggctgggctctcgacgccctgcgcgcctgctgatgcctagccagttggtaagccaggtgggcaaagaactactgcgcctggcctacagcgagccgtgcggcctgcggggggcgctgctggacgtctgcgtggagcagggcaagagctgccacagcgtgggccagctggcactcgaccccagcctggtgcccaccttccagctgaccctcgtgctgcgcctggactcacgactctggcccaagatccaggggctgtttagctccgccaactctcccttcctccctggcttcagccagtccctgacgctgagcactggcttccgagtcatcaagaagaagctgtacagctcggaacagctgctcattgaggagtgttgaacttcaacctgagggggccgacagtgccctccaagacagagacgactgaacttttggggtggagactagaggcaggagctgagggactgattcctgtggttggaaaactgaggcagccacctaaggtggaggtgggggaatagtgtttcccaggaagctcattgagttgtgtgcgggtggctgtgcattggggacacatacccctcagtactgtagcatgaaacaaaggcttaggggccaacaaggcttccagctggatgtgtgtgtagcatgtaccttattatttttgttactgacagttaacagtggtgtgacatccagagagcagctgggctgctcccgccccagcccggcccagggtgaaggaagaggcacgtgctcctcagagcagccggagggaggggggaggtcggaggtcgtggaggtggtttgtgtatcttactggtctgaagggaccaagtgtgtttgttgtttgttttgtatcttgtttttctgatcggagcatcactactgacctgttgtaggcagctatcttacagacgcatgaatgtaagagtaggaaggggtgggtgtcagggatcacttgggatctttgacacttgaaaaattacacctggcagctgcgtttaagccttcccccatcgtgtactgcagagttgagctggcaggggaggggctgagagggtgggggctggaacccctccccgggaggagtgccatctgggtcttccatctagaactgtttacatgaagataagatactcactgttcatgaatacacttgatgttcaagtattaagacctatgcaatattttttacttttctaataaacatgtttgttaaaacaaaaaaaaaaaaaaaaaa

SEQ ID NO: 2 - protein, 232 aa, Homo sapiens
MPSLWDRFSSSSTSSSPSSLPRTPTPDRPPRSAWGSATREEGFDRSTSLESSDCESLDSSNSGFGPEEDTAYLDGVSLPDFELLSDPEDEHLCANLMQLLQESLAQARLGSRRPARLLMPSQLVSQVGKELLRLAYSEPCGLRGALLDVCVEQGKSCHSVGQLALDPSLVPTFQLTLVLRLDSRLWPKIQGLFSSANSPFLPGFSQSLTLSTGFRVIKKKLYSSEQLLIEEC

SEQ ID NO: 3 - DNA, 3495 bp, Homo sapiens
atggcccaacaagcaaatgtcggggagcttcttgccatgctggactcccccatgctgggtgtgcgggacgacgtgacagctgtctttaaagagaacctcaattctgaccgtggccctatgcttgtaaacaccttggtggattattacctggaaaccagctctcagccggcattgcacatcctgaccaccttgcaagagccacatgacaagcacctcttggacaggattaacgaatatgtgggcaaagccgccactcgtttatccatcctctcgttactgggtcatgtcataagactgcagccatcttggaagcataagctctctcaagcacctcttttgccttctttactaaaatgtctcaagatggacactgacgtcgttgtcctcacaacaggcgtcttggtgttgataaccatgctaccaatgattccacagtctgggaaacagcatcttcttgatttctttgacatttttggccgtctgtcatcatggtgcctgaagaaaccaggccacgtggcggaagtctatctcgtccatctccatgccagtgtgtacgcactctttcatcgcctttatggaatgtacccttgcaacttcgtctcctttttgcgttctcattacagtatgaaagaaaacctggagacttttgaagaagtggtcaagccaatgatggagcatgtgcgaattcatccggaattagtgactggatccaaggaccatgaactggaccctcgaaggtggaagagattagaaactcatgatgttgtgatcgagtgtgccaaaatctctctggatcccacagaagcctcatatgaagatggctattctgtgtctcaccaaatctcagcccgctttcctcatcgttcagccgatgtcaccaccagcccttatgctgacacacagaatagctatgggtgtgctacttctaccccttactccacgtctcggctgatgttgttaaatatgccagggcagctacctcagactctgagttccccatcgacacggctgataactgaaccaccacaagctactctttggagcccatctatggtttgtggtatgaccactcctccaacttctcctggaaatgtcccacctgatctgtcacacccttacagtaaagtctttggtacaactgcaggtggaaaaggaactcctctgggaaccccagcaacctctcctcctccagccccactctgtcattcggatgactacgtgcacatttcactcccccaggccacagtcacaccccccaggaaggaagagagaatggattctgcaagaccatgtctacacagacaacaccatcttctgaatgacagaggatcagaagagccacctggcagcaaaggttctgtcactctaagtgatcttccagggtttttaggtgatctggcctctgaagaagatagtattgaaaaagataaagaagaagctgcaatatctagagaactttctgagatcaccacagcagaggcagagcctgtggttcctcgaggaggctttgactctcccttttaccgagacagtctcccaggttctcagcggaagacccactcggcagcctccagttctcagggcgccagcgtgaaccctgagcctttacactcctccctggacaagcttgggcctgacacaccaaagcaagcctttactcccatagacctgccctgcggcagtgctgatgaaagccctgcgggagacagggaatgccagacttctttggagaccagtatcttcactcccagtccttgtaaaattccacctccgacgagagtgggctttggaagcgggcagcctcccccgtatgatcatctttttgaggtggcattgccaaagacagcccatcattttgtcatcaggaagactgaggagctgttaaagaaagcaaaaggaaacacagaggaagatggtgtgccctctacctccccaatggaagtgctggacagactgatacagcagggagcagacgcgcacagcaaggagctgaacaagttgcctttacccagcaagtctgtcgactggacccactttggaggctctcctccttcagatgagatccgcaccctccgagaccagttgcttttactgcacaaccagttactctatgagcgttttaagaggcagcagcatgccctccggaacaggcggctcctccgcaaggtgatcaaagcagcagctctggaggaacataatgctgccatgaaagatcagttgaagttacaagagaaggacatccagatgtggaaggttagtctgcagaaagaacaagctagatacaatcagctccaggagcagcgtgacactatggtaaccaagctccacagccagatcagacagctgcagcatgaccgagaggaattctacaaccagagccaggaattacagacgaagctggaggactgcaggaacatgattgcggagctgcggatagaactgaagaaggccaacaacaaggtgtgtcacactgagctgctgctcagtcaggtttcccaaaagctctcaaacagtgagtcggtccagcagcagatggagttcttgaacaggcagctgttggttcttggggaggtcaacgagctctatttggaacaactgcagaacaagcactcagataccacaaaggaagtagaaatgatgaaagccgcctatcggaaagagctagaaaaaaacagaagccatgttctccagcagactcagaggcttgatacctcccaaaaacggattttggaactggaatctcacctggccaagaaagaccaccttcttttggaacagaagaaatatctagaggatgtcaaactccaggcaagaggacagctgcaggccgcagagagcaggtatgaggctcagaaaaggataacccaggtgtttgaattggagatcttagatttatatggcaggttggagaaagatggcctcctgaaaaaacttgaagaagaaaaagcagaagcagctgaagcagcagaagaaaggcttgactgttgtaatgacgggtgctcagattccatggtagggcacaatgaagaggcatctggccacaacggtgagaccaagacccccaggcccagcagcgcccggggcagtagtggaagcagaggtggtggaggcagcagcagcagcagcagcgagctttctaccccagagaaacccccacaccagagggcaggcccattcagcagtcggtgggagacgactatgggagaagcgtctgccagcatccccaccactgtgggctcacttcccagttcaaaaagcttcctgggtatgaaggctcgagagttatttcgtaataagagcgagagccagtgtgatgaggacggcatgaccagtagcctttctgagagcctaaagacagaactgggcaaagacttgggtgtggaagccaagattcccctgaacctagatggccctcacccgtctcccccgaccccggacagtgttggacagctacatatcatggactacaatgagactcatcatgaacacagctaa

SEQ ID NO: 4 - protein, 1164 aa, Homo sapiens
MAQQANVGELLAMLDSPMLGVRDDVTAVFKENLNSDRGPMLVNTLVDYYLETSSQPALHILTTLQEPHDKHLLDRINEYVGKAATRLSILSLLGHVIRLQPSWKHKLSQAPLLPSLLKCLKMDTDVVVLTTGVLVLITMLPMIPQSGKQHLLDFFDIFGRLSSWCLKKPGHVAEVYLVHLHASVYALFHRLYGMYPCNFVSFLRSHYSMKENLETFEEVVKPMMEHVRIHPELVTGSKDHELDPRRWKRLETHDVVIECAKISLDPTEASYEDGYSVSHQISARFPHRSADVTTSPYADTQNSYGCATSTPYSTSRLMLLNMPGQLPQTLSSPSTRLITEPPQATLWSPSMVCGMTTPPTSPGNVPPDLSHPYSKVFGTTAGGKGTPLGTPATSPPPAPLCHSDDYVHISLPQATVTPPRKEERMDSARPCLHRQHHLLNDRGSEEPPGSKGSVTLSDLPGFLGDLASEEDSIEKDKEEAAISRELSEITTAEAEPVVPRGGFDSPFYRDSLPGSQRKTHSAASSSQGASVNPEPLHSSLDKLGPDTPKQAFTPIDLPCGSADESPAGDRECQTSLETSIFTPSPCKIPPPTRVGFGSGQPPPYDHLFEVALPKTAHHFVIRKTEELLKKAKGNTEEDGVPSTSPMEVLDRLIQQGADAHSKELNKLPLPSKSVDWTHFGGSPPSDEIRTLRDQLLLLHNQLLYERFKRQQHALRNRRLLRKVIKAAALEEHNAAMKDQLKLQEKDIQMWKVSLQKEQARYNQLQEQRDTMVTKLHSQIRQLQHDREEFYNQSQELQTKLEDCRNMIAELRIELKKANNKVCHTELLLSQVSQKLSNSESVQQQMEFLNRQLLVLGEVNELYLEQLQNKHSDTTKEVEMMKAAYRKELEKNRSHVLQQTQRLDTSQKRILELESHLAKKDHLLLEQKKYLEDVKLQARGQLQAAESRYEAQKRITQVFELEILDLYGRLEKDGLLKKLEEEKAEAAEAAEERLDCCNDGCSDSMVGHNEEASGHNGETKTPRPSSARGSSGSRGGGGSSSSSSELSTPEKPPHQRAGPFSSRWETTMGEASASIPTTVGSLPSSKSFLGMKARELFRNKSESQCDEDGMTSSLSESLKTELGKDLGVEAKIPLNLDGPHPSPPTPDSVGQLHIMDYNETHHEHS

SEQ ID NO: 5 - DNA, 5424 bp, Homo sapiens
atggccaaaccaacaagcaaagattcaggcttgaaggagaagtttaagattctgttgggactgggaacaccgaggccaaatcccaggtctgcagagggtaaacagacggagtttatcatcaccgcggaaatactgagagaactgagcatggaatgtggcctcaacaatcgcatccggatgatagggcagatttgtgaagtcgcaaaaaccaagaaatttgaagagcacgcagtggaagcactctggaaggcggtcgcggatctgttgcagccggagcggacgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgtttccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcaacagctccagaccttggacagcccggagctcaggaccatcgtccatgacctgttgaccacggtggaggagctgtgtgaccagaacgagttccacgggtctcaggagagatactttgaactggtggagagatgtgcggaccagaggcctgagtcctccctcctgaacctgatctcctatagagcgcagtccatccacccggccaaggacggctggattcagaacctgcaggcgctgatggagagattcttcaggagcgagtcccgaggcgccgtgcgcatcaaggtgctggacgtgctgtcctttgtgctgctcatcaacaggcagttctatgaggaggagctgattaactcagtggtcatctcgcagctctcccacatccccgaggataaagaccaccaggtccgaaagctggccacccagttgctggtggacctggcagagggctgccacacacaccacttcaacagcctgctggacatcatcgagaaggtgatggcccgctccctctccccacccccggagctggaagaaagggatgtggccgcatactcggcctccttggaggatgtgaagacagccgtcctggggcttctggtcatccttcagaccaagctgtacaccctgcctgcaagccacgccacgcgtgtgtatgagatgctggtcagccacattcagctccactacaagcacagctacaccctgccaatcgcgagcagcatccggctgcaggcctttgacttcctgtttctgctgcgggccgactcactgcaccgcctgggcctgcccaacaaggatggagtcgtgcggttcagcccctactgcgtctgcgactacatggagccagagagaggctctgagaagaagaccagcggccccctttctcctcccacagggcctcctggcccggcgcctgcaggccccgccgtgcggctggggtccgtgccctactccctgctcttccgcgtcctgctgcagtgcttgaagcaggagtctgactggaaggtgctgaagctggttctgggcaggctgcctgagtccctgcgctataaagtgctcatctttacttccccttgcagtgtggaccagctgtgctctgctctctgctccatgctttcaggcccaaagacactggagcggctccgaggcgccccagaaggcttctccagaactgacttgcacctggccgtggttccagtgctgacagcattaatctcttaccataactacctggacaaaaccaaacagcgcgagatggtctactgcctggagcagggcctcatccaccgctgtgccagacagtgcgtcgtggccttgtccatctgcagcgtggagatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtcttttgatgacacccccgagaaggacagcttcagggcccggagtactagtctcaacgagagacccaagagtctgaggatagccagaccccccaaacaaggcttgaataactctccacccgtgaaagaattcaaggagagctctgcagccgaggccttccggtgccgcagcatcagtgtgtctgaacatgtggtccgcagcaggatacagacgtccctcaccagtgccagcttggggtctgcagatgagaactccgtggcccaggctgacgatagcctgaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggcctggactcgggggagctgcagtccggcccggagtcgagctccagccccggggtgcatgtgagacagaccaaggaggcgccggccaagctggagtcccaggctgggcagcaggtgtcccgtggggcccgggatcgggtccgttccatgtcggggggccatggtcttcgagttggcgccctggacgtgccggcctcccagttcctgggcagtgccacttctccaggaccacggactgcaccagccgcgaaacctgagaaggcctcagctggcacccgggttcctgtgcaggagaagacgaacctggcggcctatgtgcccctgctgacccagggctgggcggagatcctggtccggaggcccacagggaacaccagctggctgatgagcctggagaacccgctcagccctttctcctcggacatcaacaacatgcccctgcaggagctgtctaacgccctcatggcggctgagcgcttcaaggagcaccgggacacagccctgtacaagtcactgtcggtgccggcagccagcacggccaaaccccctcctctgcctcgctccaacacagtggcctctttctcctccctgtaccagtccagctgccaaggacagctgcacaggagcgtttcctgggcagactccgccgtggtcatggaggagggaagtccgggcgaggttcctgtgctggtggagcccccagggttggaggacgttgaggcagcgctaggcatggacaggcgcacggatgcctacagcaggtcgtcctcagtctccagccaggaggagaagtcgctccacgcggaggagctggttggcaggggcatccccatcgagcgagtcgtctcctcggagggtggccggccctctgtggacctctccttccagccctcgcagcccctgagcaagtccagctcctctcccgagctgcagactctgcaggacatcctcggggaccctggggacaaggccgacgtgggccggctgagccctgaggttaaggcccggtcacagtcagggaccctggacggggaaagtgctgcctggtcggcctcgggcgaagacagtcggggccagcccgagggtcccttgccttccagctccccccgctcgcccagtggcctccggccccgaggttacaccatctccgactcggccccatcacgcaggggcaagagagtagagagggacgccttaaagagcagagccacagcctccaatgcagagaaagtgccaggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagcctacctctggtgcaccctccgtcccatagcaaagcccctgcacagactccagccgagcccacacctggctatgaggtgggccagcggaagcgcctcatctcctcggtggaggacttcaccgagtttgtgtga

SEQ ID NO: 6 - protein, 1807 aa, Homo sapiens
MAKPTSKDSGLKEKFKILLGLGTPRPNPRSAEGKQTEFIITAEILRELSMECGLNNRIRMIGQICEVAKTKKFEEHAVEALWKAVADLLQPERTLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVFPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMERFFRSESRGAVRIKVLDVLSFVLLINRQFYEEELINSVVISQLSHIPEDKDHQVRKLATQLLVDLAEGCHTHHFNSLLDIIEKVMARSLSPPPELEERDVAAYSASLEDVKTAVLGLLVILQTKLYTLPASHATRVYEMLVSHIQLHYKHSYTLPIASSIRLQAFDFLFLLRADSLHRLGLPNKDGVVRFSPYCVCDYMEPERGSEKKTSGPLSPPTGPPGPAPAGPAVRLGSVPYSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCARQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSFDDTPEKDSFRARSTSLNERPKSLRIARPPKQGLNNSPPVKEFKESSAAEAFRCRSISVSEHVVRSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTVASFSSLYQSSCQGQLHRSVSWADSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 7 - DNA, 1356 bp, Homo sapiens
atgcgtgagtgcatctccatccacgttggccaggctggtgtccagattggcaatgcctgctgggagctctactgcctggaacacggcatccagcccgatggccagatgccaagtgacaagaccattgggggaggagatgactccttcaacaccttcttcagtgagacgggcgctggcaagcacgtcccccgggctgtgtttgtagacttggaacccacagtcattgatgaagttcgcactggcacctaccgccagctcttccaccctgagcagctcatcacaggcaaggaagatgctgccaataactatgcccgagggcactacaccattggcaaggagatcattgaccttgtgttggaccgaattcgcaagctggctgaccagtgcaccggtcttcagggcttcttggttttccacagctttggtgggggaactggttctgggttcacctccctgctcatggaacgtctctcagttgattatggcaagaagtccaagctggagttctccatttacccagcaccccaggtttccacagctgtagttgagccctacaactccatcctcaccacccacaccaccctggagcactctgattgtgccttcatggtagacaatgaggccatctatgacatctgtcgtagaaacctcgatatcgagcgcccaacctacactaaccttaaccgccttattagccagattgtgtcctccatcactgcttccctgagatttgatggagccctgaatgttgacctgacagaattccagaccaacctggtgccctacccccgcatccacttccctctggccacatatgcccctgtcatctctgctgagaaagcctaccatgaacagctttctgtagcagagatcaccaatgcttgctttgagccagccaaccagatggtgaaatgtgaccctcgccatggtaaatacatggcttgctgcctgttgtaccgtggtgacgtggttcccaaagatgtcaatgctgccattgccaccatcaaaaccaagcgcagcatccagtttgtggattggtgccccactggcttcaaggttggcatcaactaccagcctcccactgtggtgcctggtggagacctggccaaggtacagagagctgtgtgcatgctgagcaacaccacagccattgctgaggcctgggctcgcctggaccacaagtttgacctgatgtatgccaagcgtgcctttgttcactggtacgtgggtgaggggatggaggaaggcgagttttcagaggcccgtgaagatatggctgcccttgagaaggattatgaggaggttggtgtggattctgttgaaggagagggtgaggaagaaggagaggaatactaa

SEQ ID NO: 8 - protein, 451 aa, Homo sapiens [sequence omitted]

SEQ ID NO: 9 - DNA, 2607 bp, Homo sapiens
agccggcgcagggtggccggggaggggtgagcagggtgccgctggctgctggggtctgcaggtcaccgagtccccaggagaggggactcctaagaagccacctgcctgtgtttacccggcagcgagcgcgcaggcccccgcgaactcctggcagcgctcaggaaaggccgttgcgcctcgcgaaggaaacagagccgttgaccatggttgcaactggcagtttgagcagcaagaacccggccagcatttcagaattgctggactgtggctatcacccagagagcctgctaagtgattttgactactgggattatgttgttcctgaacccaacctcaacgaggtaatatttgaggaatcaacttgccagaatttggttaaaatgctggagaactgtctgtccaaatcaaagcaaactaaacttggttgctcaaaggtccttgtccctgagaaactgacccagagaattgctcaagatgtcctgcggctttcctcaacggagccctgcggcttgcgaggttgtgttatgcacgtgaacttggaaattgaaaatgtatgtaaaaagctggataggattgtgtgtgattctagcgtcgtacctacttttgagcttacacttgtgtttaagcaggagaactgctcatggactagcttcagggactttttctttagtagaggtcgcttctcctctggtttcaggagaactctgatcctcagctcaggatttcgacttgttaagaaaaaactttactcactgattggaacaacagtgattgaagggtcctaaaaagggaaaatatataaagattatttcatgattgggtagtaaaactattcagctagtcagctaaagtcatttgtagtttgccccacctgccctaaataagaaaccccaaatgtagtctcttttctttctgtgtttcacattcatagcaactgcagctaacaggctgattttctggcctttggagaagtgattcaaaatagtgtagattttctgcatagatcccatttttgtacagaattgaatgggatggaataggtaagcaaaagtagaagcccatttgagttttacatttgattccacaatttggtttcaggtaggcttggtaatagactatataaaccagatttgcctattttgattttcatatggcttttttttctctaagttttcagaggattttttaaatcacagaatcatactaaatgatatttagcctatcaaaacttccaaaagcccacaccaccagttcctgactcaaatttgaagggtttttagacaggaaggtaggattaagtaggtgagtttaattaaagcttaaccctaggtaagagtaaatgagaaatattacggcaataatggaactgcttcactgtttcttggtgacttcctcactctaatgttttaaagaggcaacaaaagcttgtggtgccatttcagtaaccacggtgttgttttagatgcctttataagctcagtttcccctgttcttaagtgttgaatactgtctttaaactagaaaaatgcaaaatattgaactgatatttttgtgtgtagttgattactcttccattgagtgaatgatgaatacctgtgaggataggaaattagttctgagatctagtccctctctgattcacttagtaatctatcctcttttcagtattacatgtgcttaatctcagatgaaccatttcaccatggcagtgttatctcatctctgggcttttctgggaattgaagtatctctccttaaccccaattgtcaagggtagtagctgtatactaccactttgaattattgaaacgggtcaatttacgaagtctgcattggctatggagatatggtttatagtacagcctagagaatgaaactcaccgtccagataaccatgcatgcacccagattttttccaccttggatacctgtcactagggaataataaaggccttattttttgtcttattccaactaagtagatcattatctctttccttttttatgttaatgagagaatttagcctccactcaacaatgttcaattcagcaaggctttcatatccttgctgtgggtcgtggataaggagcttattcaggtttcctgccctagctattagctccacttcacatgctggagaccggcgtagggacagatgtattcatcctggtgttactgaaaaacaggtgtgatcctgttactgatactataagtgacctaaaatgtcactgttcaaattagccagtgttctaacaaactaaactcttcaaatgcttggaaagatactacaaagccaatctttatagaattgggccaagataaatcaatgttgttttgcatgtctattgttaagctccaaaggttcactgtgtttctgccgctgtcctggagttgtcaccactgactgggcaaggcttcttgggcatcgatgtagaactgttgtcctttttccactaacagttatctttgactctcttgcctgttatgcttacaaaatggtgatggcttatggaaggctgttaaattaatattcctgttaaaggaaattaaagtttgtctatttttgacaataaaacattatatatttttaaaaaaaaaaaaaaaaa

SEQ ID NO: 10 - protein, 193 aa, Homo sapiens
MVATGSLSSKNPASISELLDCGYHPESLLSDFDYWDYVVPEPNLNEVIFEESTCQNLVKMLENCLSKSKQTKLGCSKVLVPEKLTQRIAQDVLRLSSTEPCGLRGCVMHVNLEIENVCKKLDRIVCDSSVVPTFELTLVFKQENCSWTSFRDFFFSRGRFSSGFRRTLILSSGFRLVKKKLYSLIGTTVIEGS

(19) United States
(12) Patent Application Publication
(10) Pub. No.: US 2008/0014599 A1
(43) Pub. Date: Jan. 17, 2008

(54) Screening systems utilizing RTP801L

(76) Inventors: Roni Wechsler, Shoham (IL); Igor Mett, Rehovot (IL)

Correspondence Address: COOPER & DUNHAM, LLP, 1185 AVENUE OF THE AMERICAS, NEW YORK, NY, 10036, US

(21) Appl. No.: 11/823,823

(22) Filed: Jun. 27, 2007

Related U.S. Application Data
(60) Provisional application No. 60/855,101, filed on Oct. 26, 2006.
(60) Provisional application No. 60/817,258, filed on Jun. 28, 2006.

(51) Int. Cl. G01N 33/567

(52) U.S. Cl. 435/7.21; 436/501

(57) ABSTRACT

RTP801L represents a unique gene target for hypoxia-inducible factor-1 (HIF-1) that may regulate hypoxia-induced pathogenesis; down-regulation of the mTOR pathway activity by hypoxia requires de novo mRNA synthesis and correlates with increased expression of RTP801L.

The present invention relates to screening systems utilizing RTP801L and/or RTP801L interactors and/or RTP801L biological activity, and to the potential drugs and methods of treatment identified by such screening systems.

This application claims priority of U.S. Provisional patent applications No. 60/817,258, filed Jun. 28, 2006 and No. 60/855,101, filed 26-Oct. 26, 2006, both of which are hereby incorporated by reference in their entirety.

Throughout this application, various publications, including United States patents, are referenced by author and year and patents by number. The disclosures of these publications and patents and patent applications in their entireties are hereby incorporated by reference into this application in order to more fully describe the state of the art to which this invention pertains.

### FIELD OF THE INVENTION

The present invention relates to novel screening systems utilizing RTP801L, and to the use of molecules identified by such screening systems to treat neurodegenerative diseases, respiratory disorders of all types (including pulmonary disorders), eye diseases and conditions, microvascular disorders, angiogenesis- and apoptosis-related conditions, neurodegenerative diseases and hearing impairments.

### BACKGROUND OF THE INVENTION

Current modes of therapy for the prevention and/or treatment of apoptosis-related and neurodegenerative diseases, ischemic conditions, COPD, macular degeneration, microvascular diseases and ototoxic conditions are unsatisfactory and there is a need therefore to develop novel compounds for this purpose. The present invention is focused on processes for identifying such compounds. All the diseases and indications disclosed herein, as well as other diseases and conditions disclosed in PCT Application Publication No. WO06/023544A2, assigned to the assignee of the present invention, may also be treated by the novel compounds of this invention.

RTP801L

Gene RTP801 was first reported by the assignee of the instant application. U.S. Pat. Nos. 6,455,674, 6,555,667, and 6,740,738, all assigned to the assignee of the instant application, disclose and claim per se the RTP801 polynucleotide and polypeptide, and antibodies directed toward the polypeptide. RTP801 represents a unique gene target for hypoxia-inducible factor-1 (HIF-1) that may regulate hypoxia-induced pathogenesis independent of growth factors such as VEGF. Further discoveries relating to gene RTP801, as discovered by the assignee of the instant application, were reported in: Tzipora Shoshani, et al. Identification of a Novel Hypoxia-Inducible Factor 1-Responsive Gene, RTP801, Involved in Apoptosis. MOLECULAR AND CELLULAR BIOLOGY, April 2002, p. 2283-2293; this paper, co-authored by the inventor of the present invention, details the discovery of the RTP801 gene. Gene RTP801L, so named because of its resemblance to RTP801, was also first reported by the assignee of the instant application, and given Pubmed accession No. NM_{—}145244.

It has been demonstrated that RTP801/REDD1 and RTP801L/REDD2 potently inhibit signaling through mTOR, by working downstream of AKT and upstream of TSC2 to inhibit mammalian target of rapamycin (mTOR) functions. mTOR is a serine/threonine kinase that plays an essential role in cell growth control. mTOR stimulates cell growth by phosphorylating p70 ribosomal S6 kinase (S6K) and eukaryote initiation factor 4E-binding protein 1 (4EBP1). The mTOR pathway is regulated by a wide variety of cellular signals, including mitogenic growth factors, nutrients, cellular energy levels, and stress conditions. (Corradetti et al, The stress-inducted proteins RTP801 and RTP801L are negative regulators of the mammalian target of rapamycin pathway. J Biol Chem. Mar. 18, 2005 18;280(11):9769-72. Epub Jan. 4, 2005.)

Also reported under the name “SMHS1”, RTP801L was found to be upregulated in rat soleus muscle atrophied by restriction of activity. (Pisani et al., SMHS1 is involved in oxidative/glycolytic-energy metabolism balance of muscle fibers. Biochem Biophys Res Commun Jan. 28, 2005;326(4):788-93.). While the RTP801L amino acid sequence shares 65% similarity with RTP801—which is a cellular stress response protein regulated by HIF-1, RTP801L expression was demonstrated to be independent of HIF-1. RTP801L was found to be mainly expressed in skeletal muscle, and comparisons of its expression in atrophied versus hypertrophied muscles and in oxidative versus glycolytic muscles suggested that RTP801L contributes to the muscle energy metabolism phenotypes.

Further, the RTP801L gene was found to be was strongly up-regulated as THP-1 macrophages are converted to foam cells. Treatment of HMDM with desferrioxamine, a molecule that mimics the effect of hypoxia, increased expression of RTP801L in a concentration-dependent fashion. Transfection of U-937 and HMEC cells with a RTP801L expression vector increased the sensitivity of the cells for oxLDL-induced cytotoxicity, by inducing a shift from apoptosis toward necrosis. In contrast, suppression of mRNA expression using siRNA approach resulted in increased resistance to oxLDL treatment. Thus, it has been demonstrated that stimulation of RTP801L expression in macrophages increases oxLDL-induced cell death, suggesting that RTP801L gene might play an important role in arterial pathology. (Cuaz-perolin et al., REDD2 gene is upregulated by modified LDL or hypoxia and mediates human macrophage cell death. Arterioscler Thromb Vase Biol. 2004 October;24(10):1830-5. Epub Aug. 12, 2004).

Additionally, Sofer et al (Regulation of mTOR and cell growth in response to energy stress by REDD1.; Mol Cell Biol. 2005 July;25(14):5834-45.) have shown that RTP801 and RTP801L have non-overlapping expression patterns in adult tissues, and that RTP801L mRNA is absent in immortalized MEFs ± Glucose and 2DG, thus demonstrating that RTP801 may function independently of RTP801L.

While RTP801 and RTP801L share sequence homology of about 65% at the amino acid level, indicating a possible similarity of function, and while the assignee of the present invention has found that both RTP801 and RTP801L interact with TSC2 and affect the mTOR pathway, the inventors of the present invention have found that the embryological expression pattern of the two polypeptides differs, and that, contrary to RTP801, RTP801L is not induced by hypoxia in all conditions which induce RTP801 expression; it is, however, induced in MEFs as a result of H2O2 treatment (hypoxia treatment), and the induction follows kinetics similar to those of RTP801 expression induction under the same conditions. Additionally, the inventors of the present invention have found that RTP801 polypeptide is more abundantly expressed than RTP801L. Thus, RTP801L may be used as a target in the treatment of conditions for which RTP801 is a target, and may have the added benefit of a similar—yet different—target.

Without being bound by theory, RTP801L may be a factor acting in fine-tuning of cell response to energy disbalance. As such, it is a target suitable for treatment of any disease where cells should be rescued from apoptosis due to stressful conditions (e.g. diseases accompanied by death of normal cells) or where cells, which are adapted to stressful conditions due to changes in RTP801L expression (e.g. cancer cells), should be killed. In the latter case, RTP801L may be viewed as a survival factor for cancer cells and its inhibitors may treat cancer as a monotherapy or as sensitising drugs in combination with chemotherapy or radiotherapy. The assignee of the present invention has previously discovered gene RTP801 (see above) and molecules effective in inhibiting gene RTP801 (see co-assigned PCT publication No. WO06/023544A2 and PCT Application No. PCT/US2007/01468, hereby incorporated by reference in their entirety). Although RTP801L shares sequence and functional homology with RTP801, the assignee of the present invention has discovered that inhibition of RTP801 does not cause simultaneous inhibition of RTP801L, and vice versa. Therefore, RTP801L is an excellent target for inhibition in the conditions disclosed herein, and its inhibition is gene-specific. Tandem therapies which inhibit both RTP801 and RTP801L can have additional advantages and are discussed herein below.

The following patent applications and publications give aspects of background information:

Patent application/publication Nos EP1580263, WO2003029271, WO2001096391, WO2003087768, WO2004048938, WO2005044981, WO2003025138, WO2002068579, EP1104808 and CA2343602 all disclose a nucleic acid or polypeptide which is homologous to RTP801L.

Tzipora Shoshani, et al. Identification of a Novel Hypoxia-Inducible Factor 1-Responsive Gene, RTP801, Involved in Apoptosis. MOLECULAR AND CELLULAR BIOLOGY, April 2002, p. 2283-2293. This paper, co-authored by the inventor of the present invention, details the discovery of the RTP801 gene.

Anat Brafman, et al. Inhibition of Oxygen-Induced Retinopathy in RTP801 check!!—Deficient Mice. Invest Ophthalmol Vis Sci. 2004 October; 45 (10): 3796-805; also co-authored by the inventor of the present invention, this paper demonstrates that in RTP801 knock out mice, hyperoxia does not cause degeneration of the retinal capillary network.

Leif W. Ellisen, et al. REDD1, a Developmentally Regulated Transcriptional Target of p63 and p53, Links p63 to Regulation of Reactive Oxygen Species. Molecular Cell, Vol. 10, 995-1005, November, 2002;this paper demonstrates that overexpression of RTP801 (referred to therein as REDD1) leads to increased production of reactive oxygen species.

Richard DR, Berra E, and Pouyssegur J. Non-hypoxic pathway mediates the induction of hypoxia-inducible factor 1 alpha in vascular smooth muscle cells. J Biol. Chem. Sep. 1, 2000, ;275(35): 26765-71 this paper demonstrates that HIF-1-dependent transcription may be induced by excessive production of reactive oxygen species.

Rangasami T, et al., Genetic ablation of Nrf2 enhances susceptibility to cigarette smoke-induced emphysema in mice. Submitted to Journal of Clinical Investigation. This work relates to mice with a compromised antoxidant defence (due to a germline inactivation of RTP801).

Corradetti et al, The stress-inducted proteins RTP801 and RTP801L are negative regulators of the mammalian target of rapamycin pathway. J Biol Chem. Mar. 18, 2005;280(11):9769-72. Epub Jan. 4, 2005.

Pisani et al., SMHS1 is involved in oxidative/glycolytic-energy metabolism balance of muscle fibers. Biochem Biophys Res Commun Jan. 28, 2005;326(4):788-93.). Cuaz-perolin et al., REDD2 gene is upregulated by modified LDL or hypoxia and mediates human macrophage cell death. Arterioscler Thromb Vasc Biol. 2004 October;24(10):1830-5. [Epub Aug. 12, 2004.).

Sofer et al., Regulation of mTOR and cell growth in response to energy stress by REDD1. Mol Cell Biol. 2005 July;25(14):5834-45.

The mTOR Pathway

Tuberous sclerosis is an autosomal-dominant disorder caused by the mutation of one of the two tumor suppressor genes: TSC1 or TSC2, (TSC=Tuberous Sclerosis Complex) encoding protein products, hamartin, and tuberin, respectively. Both proteins form intracellular complexes exerting inhibitory activity on mammalian target of rapamycin (mTOR) kinase. It has been demonstrated that signal transduction from tuberin to mTOR is mediated by a G protein, Ras homologue enriched in brain (Rheb). In normal cells, tuberin i5 having GTPase-activating protein properties toward Rheb controls signals of nutrient depletion, hypoxia, or stress, not allowing activation of mTOR and subsequent protein translation and cell proliferation. However, when environmental conditions change, tuberin is phosphorylated and it forms a complex with hamartin is degraded, and downstream targets of mTOR, S6K, and eEF2K, can be activated. (Jozwiak J, Jozwiak S, Grzela T, Lazarczyk M: Positive and negative regulation of TSC2 activity and its effects on downstream effectors of the mTOR pathway. Neuromolecular Med. 2005;7(4):287-96.).

mTOR is a central regulator of protein synthesis the activity of which is modulated by a variety of signals. Energy depletion and hypoxia result in mTOR inhibition through a process involving the activation of AMP-activated protein kinase (AMPK) by LKB1 and subsequent phosphorylation of TSC2. It has been shown that mTOR inhibition by hypoxia requires the TSC1/TSC2 tumor suppressor complex and RTP801. Disruption of the TSC1/TSC2 complex through loss of TSC1 or TSC2 blocks the effects of hypoxia on mTOR, as measured by changes in the mTOR targets S6K and 4E-BP1, and results in abnormal accumulation of Hypoxia-inducible factor (HIF). In contrast to energy depletion, mTOR inhibition by hypoxia does not require AMPK or LKB1. Down-regulation of mTOR activity by hypoxia requires de novo mRNA synthesis and correlates with increased expression of RTP801. Disruption of RTP801 abrogates the hypoxia-induced inhibition of mTOR, and RTP801 overexpression is sufficient to down-regulate S6K phosphorylation in a TSC1/TSC2-dependent manner. (Brugarolas J, Lei K, Hurley R L, Manning B D, Reiling J H, Hafen E, Witters L A, Ellisen L W, Kaelin W G Jr.: Regulation of mTOR function in response to hypoxia by REDD1 and the TSC1/TSC2 tumor suppressor complex. Genes Dev. Dec. 1, 2004;18(23):2893-904.)

Additionally, it has recently been demonstrated that RTP801 potently inhibit signaling through mTOR, working downstream of AKT and upstream of TSC2 to inhibit mTOR functions. (Corradetti Minn., et al.,. J Biol Chem. Mar. 18, 2005;280(11):9769-72.).

### SUMMARY OF THE INVENTION

The present invention relates to screening systems aimed at identifying molecules which can inhibit or enhance the activity of RTP801L, thereby identifying molecules which may be used for the treatment of various diseases and conditions. Thus, in some embodiments the present invention comprises processes for-identifying a test compound useful for imodulating the activity of an RTP801L polypeptide

The present invention further provides novel methods and compositions for treating apoptotic or neurodegenerative diseases, as well as microvascular disorders, macular degeneration, respiratory disorders, and spinal cord injury or disease.

### BRIEF DESCRIPTION OF THE FIGURES

FIG. 1 details the coding sequence of the RTP801 gene (SEQ ID NO:1);

FIG. 2 details the amino acid sequence of the RTP801 polypeptide (SEQ ID NO:2);

FIG. 3 details the coding sequence of the TSC1 gene (SEQ ID NO:3);

FIG. 4 details the amino acid sequence of the TSC1 polypeptide (SEQ ID NO:4);

FIG. 5 details the coding sequence of the TSC2 gene (SEQ ID NO:5);

FIG. 6 details the amino acid sequence of the TSC2 polypeptide (SEQ ID NO:6);

FIG. 7 details the coding sequence of the alpha-tubulin gene (SEQ ID NO:7);

FIG. 8 details the amino acid sequence of the alpha-tubulin polypeptide (SEQ ID NO:8);

FIG. 9 demonstrates that ZO-1 and cingulin are up-regulated upon hypoxia treatment in RTP801 knock-down cells;

FIG. 10 discovery of alpha/beta tubulin and cytokeratin-9 as proteins that co-IP with FLAG-hRTP801—demonstrates that alpha/beta tubulin and cytokeratin-9 co-immunoprecipitate with RTP801;

FIG. 11 shows co-immunoprecipitation of exogenous TSC2 with alpha tubulin and RTP801;

FIG. 12 hRTP801 co-IP with tubulin independently of exogenous TSC2—demonstrates that RTP801 co-immunoprecipitates with tubulin independently of exogenous TSC2;

FIG. 13 binding in vitro of 6× His-hRTP801 and 6× His-hRTP801 C-fragment (but not 6× His hRTP801 N-fragment) to TSC2 (“pull-down” from extract)—shows binding in vitro of RTP801 and RTP801 C-fragment (but not RTP801 N-fragment) to TSC2;

FIG. 14 binding in vitro of GST-hRTP801 (but not of free GST to TSC2 and to tubulin). A. Input extracts used for experiment B. Pull down result—demonstrates binding in vitro of GST-hRTP801 (but not of free GST) to TSC2 and to tubulin;

FIG. 15 monoclonal anti-hRTP801 C-fragment (termed mAb “B”) abolishes binding in vitro of GST-hRTP801 to TSC2 whereas monoclonal anti-hRTP801 N-fragment (termed mAb “A”) has no effect. A. Specificity of mAbs as judged by ELISA. B. Effect of pre-incubation with mAbs “A” or “B” on binding of GST-hRTP801 to TSC2.—shows that monoclonal anti-hRTP801 C-fragment abolishes binding in vitro of GST-hRTP801 to TSC2 whereas monoclonal anti-hRTP801 N-fragment has no effect;

FIG. 16 demonstrates that binding of TSC2 to RTP801 occurs within the C-fragment while binding of alpha tubulin to hRTP801 requires both C- and N-fragments;

FIG. 17 shows that TSC2 “N” fragment (a.a. 2-935) is sufficient for interaction with FLAG-hRTP801;

FIG. 18 schematic description of suggested ELISA-based assay for discovery of small molecules that can inhibit hRTP801/TSC2 complex—depicts a schematic description of an exemplary ELISA-based assay for discovery of small molecules that can inhibit the RTP801/TSC2 complex;

FIG. 19 shows that binding of HA-tagged TSC2 to GST-hRTP801 can be detected using an ELISA-based assay;

FIG. 20 binding of purified tubulin to GST-hRTP801, GST-hRTP C-frag. and GST-hRTP801 N-frag. but not to free GST. A. Purified tubulin binds to both full hRTP801 and to its C-frag. B. Purified tubulin binds the hRTP801 N-frag.—demonstrates binding of purified tubulin of purified tubulin to RTP801;

FIG. 21 shows that full length RTP801 co-immunoprecipitated with FLAG-hRTP801, indicating self association of hRTP801;

FIG. 22 shows results obtained using various RTP801 fragments;

FIG. 23 depicts HTRF results relating to self association of hRTP801;

FIG. 24 shows the RTP801 region that binds TSC2;

FIG. 25 shows the TSC2 region that binds hRTP801;

FIG. 26 depicts an additional exemplary assay;

FIG. 27 shows reciprocal co-immunoprecipitation of exogenous RTP801 with endogenous Tyr-tubulin;

FIG. 28 shows co-immunoprecipitation of endogenous Tyr-tubulin with endogenous RTP801;

FIG. 29 depicts results indicating that RTP801 has preference for Tyr-tubulin as compared with de-tyrosinated tubulin (Glu-tubulin);

FIG. 30 presents the results of co-immunoprecepitation in a 96-well format;

FIG. 31 shows that endogenous TSC2 co-immunoprecipitated with endogenous Tyr-alpha-tubulin;

FIG. 32 demonstrates that co-immunoprecipitation of endogenous TSC2 with tubulin was significantly reduced in the presence of overexpressed exogenous RTP801;

FIG. 33 shows reduced motility of RTP801 KO mouse embryo fibroblasts;

FIG. 34 co-immunoprecipitation of FLAG-hRTP801 and FLAG-hRTP801-L with endogenous alpha tubulin and TSC2—shows that RTP801 and RTP801-L co-immunoprecipitate with endogenous alpha tubulin and TSC2;

FIG. 35 coding sequence of RTP801Like (Ddit4L) (GI:34222182), orf=nucleotides 204-785—details the coding sequence of the RTP801L gene (SEQ ID NO:9); and

FIG. 36 amino acid sequence of RTP801Like (gi:21687001)—details the amino acid sequence of the RTP801L polypeptide (SEQ ID NO:10).

### DETAILED DESCRIPTION OF THE INVENTION

The present invention relates to screening systems for identifying molecules which inhibit or enhance the activity of RTP801L, inter alia in its capacity to modulate apoptotic and/or neurotoxic conditions, as well as its capacity to affect the mTOR pathway. The inventors of the present invention have discovered that RTP801L self associates (forms homodimers or oligomers) and also binds to TSC1 and TSC2, said binding potentially affecting the mTOR pathway. The object of the present invention is therefore to identify molecules which may modulate this binding and/or the activity or self-association of RTP801L, thereby affecting to inhibition or enhancement of any of the mTOR pathway participants, resulting in molecules which may be used to treat diseases or conditions which relate to apoptosis, ischemia or anoxia, or any other disadvantageous conditions relating to the mTOR pathway or mTOR pathway malfunction.

Further, the inventors of the present invention have discovered that RTP801L binds to alpha-tubulin, particularly to tyrosinated tubulin, said binding potentially affecting RTP801L activity in any processes which relate to cellular integrity such as, inter alia, apoptosis or anoxia. Any of the diseases and conditions mentioned herein may be treated using pharmaceutical compositions comprising the molecules identified by the methods of the present invention.

RTP801L binds RTP801L (self-association/homodimerization) and/or TSC1 and/or TSC2 and/or RTP801 and may therefore, without being bound by theory, inhibit the mTOR pathway or mTOR signalling by causing or enhancing association of the TSC complex, possibly by affecting the phosphorylation state of one or more of the complex members. Without being bound by theory, it would therefore be beneficial to enhance RTP801L activity in cases where mTOR pathway inhibition is desired and inhibit RTP801L activity in cases where mTOR pathway up-regulation is desired. RTP801L can be considered as the “glue” that strengthens the TSC complex, which in turn causes down-regulation in mTOR signaling.

As stated above, RTP801L can self associate. RTP801 can also self-associate, and the self association of RTP801 has been mapped by the inventors of the present invention to a region between a.a 161-195. This region is conserved between RTP801 and RTP801L, and RTP801L self association is probably of functional significance similarly to that of RTP801 (a deletion mutant in RTP801 that lacks this region and cannot self associate, is also non-functional. In addition, a 70 a.a fragment that contains this self-association region is functionally competent).

For further information concerning the mTOR pathway and the various interactors involved in said pathway, see: Jozwiak J, Jozwiak S, Grzela T, Lazarczyk M: Positive and negative regulation of TSC2 activity and its effects on downstream effectors of the mTOR pathway. Neuromolecular Med. 2005;7(4):287-96.; Brugarolas J, Lei K, Hurley R L, Manning B D, Reiling J H, Hafen E, Witters L A, Ellisen L W, Kaelin W G Jr.: Regulation of mTOR function in response to hypoxia by REDD1 and the TSC1/TSC2 tumor suppressor complex. Genes Dev. Dec. 1, 2004;18(23):2893-904.; Sofer A, Lei K, Johannessen C M, Ellisen L W.: Regulation of mTOR and cell growth in response to energy stress by REDD1. Mol Cell Biol. 2005 July;25(14):5834-45.; Corradetti Minn., Inoki K, Guan K L: The stress-inducted proteins RTP801 and RTP801L are negative regulators of the mammalian target of rapamycin pathway. J Biol Chem. Mar. 18, 2005;280(11):9769-72.

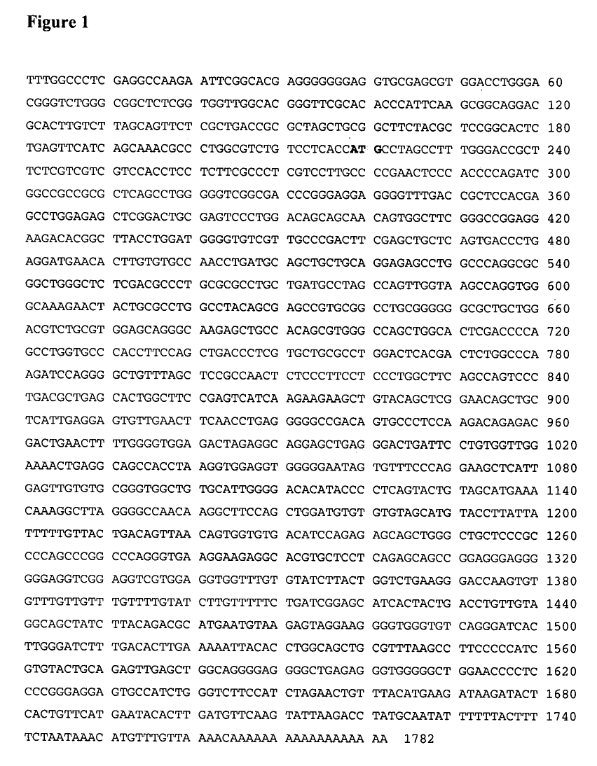

“RTP801 gene” refers to the RTP801 coding sequence open reading frame, as shown in FIG. 1 (SEQ ID NO:1), or any homologous sequence thereof preferably having at least 70% identity, more preferable 80% identity, even more preferably 90% or 95% identity. This encompasses any sequences derived from SEQ ID NO:1 which have undergone mutations, alterations or modifications as described herein. Thus, in a preferred embodiment RTP801 is encoded by a nucleic acid sequence according to SEQ. ID. NO. 1. It is also within the present invention that the nucleic acids according to the present invention are only complementary and identical, respectively, to a part of the nucleic acid coding for RTP801 as, preferably, the first stretch and first strand is typically shorter than the nucleic acid according to the present invention. It is also to be acknowledged that based on the amino acid sequence of RTP801 any nucleic acid sequence coding for such amino acid sequence can be perceived by the one skilled in the art based on the genetic code.

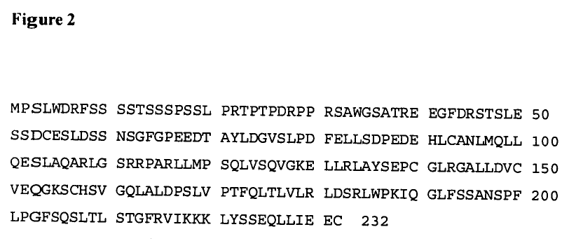

“RTP801 polypeptide” refers to the polypeptide of the RTP801 gene, and is understood to include, for the purposes of the instant invention, the terms “RTP779”, “REDD1”, “Ddit4”, “FLJ20500”, “Dig2”, and “PRF1”, derived from any organism, optionally man, splice variants and fragments thereof retaining biological activity (such as the functional fragments disclosed herein), and homologs thereof, preferably having at least 70%, more preferably at least 80%, even more preferably at least 90% or 95% homology thereto. In addition, this term is understood to encompass polypeptides resulting from minor alterations in the RTP801 coding sequence, such as, inter alia, point mutations, substitutions, deletions and insertions which may cause a difference in a few amino acids between the resultant polypeptide and the naturally occurring RTP801. Polypeptides encoded by nucleic acid sequences which bind to the RTP801 coding sequence or genomic sequence under conditions of highly stringent hybridization, which are well-known in the art (for example Ausubel et al., Current Protocols in Molecular Biology, John Wiley and Sons, Baltimore, Md. (1988), updated in 1995 and 1998), are also encompassed by this term. Chemically modified RTP801 or chemically modified fragments of RTP801 are also included in the term, so long as the biological activity is retained. RTP801 preferably has or comprises an amino acid sequence according to SEQ. ID. NO. 2. It is acknowledged that there might be differences in the amino acid sequence among various tissues of an organism and among different organisms of one species or among different species to which the nucleic acid according to the present invention can be applied in various embodiments of the present invention. However, based on the technical teaching provided herein, the respective sequence can be taken into consideration accordingly when designing any of the nucleic acids according to the present invention. Particular fragments of RTP801 include amino acids 1-50, 51-100,101-150, 151-200 and 201-232 of the sequence shown in FIG. 2. Further particular fragments of RTP801 include amino acids 25-74, 75-124, 125-174, 175-224 and 225-232 of the sequence shown in FIG. 2. The inventors of the present invention have discovered that RTP801 binds itself (see Example 5), and this can also be used in the screening methods of the present invention, enabling search for molecules or agents which can inhibit or enhance binding of RTP801 to itself, as described herein. IZTP801 as used herein is a protein described, among others, in WO 99/09046. RTP801 has been described as a transcriptional target of HIF-1 by Shoshani T et al. (Shoshani et al., 2002, Mol Cell Biol, 22, 2283-93). Furthermore the study by Ellisen et al. (Ellisen et al., Mol Cell, 10, 995-1005) has identified RTP801 as a p53-dependent DNA damage response gene and as a p63-dependent gene involved in epithelial differentiation. Also, RTP801 mirrors the tissue-specific pattern of the p53 family member p63, is effective similar to or in addition to TP 63, is an inhibitor to in vitro differentiation, and is involved in the regulation of reactive oxygen species. Apart from that, RTP801 is responsive to hypoxia-responsive transcription factor hypoxia-inducible factor 1 (HIF-1) and is typically up-regulated during hypoxia both in vitro and in vivo in an animal model of ischemic stroke. RTP801 appears to function in the regulation of reactive oxygen species (ROS) and ROS levels and reduced sensitivity to oxidative stress are both increased following ectopic expression RTP801 (Ellisen et al. 2002, supra; Soshani et al. 2002, supra). Preferably, RTP801 is a biologically active RTP801 protein which preferably exhibits at least one of those characteristics, preferable two or more and most preferably each and any of these characteristics. For the purposes of the present invention, RTP801 activity can also be defined as the ability of RTP801 to form a complex with a polypeptide, such as, inter alia, itself, TSC1, TSC2 or alpha-tubulin. Without being bound by theory, any polypeptide RTP801 forms a complex with may be involved in exerting the activity RTP801 has on various signal transduction pathways. Thus, a compound that disturbs the complex formation of RTP801 and a polypeptide such as inter alia, RTP801, TSC1, TSC2 or alpha-tubulin, is a compound which modulates the activity of RTP801.

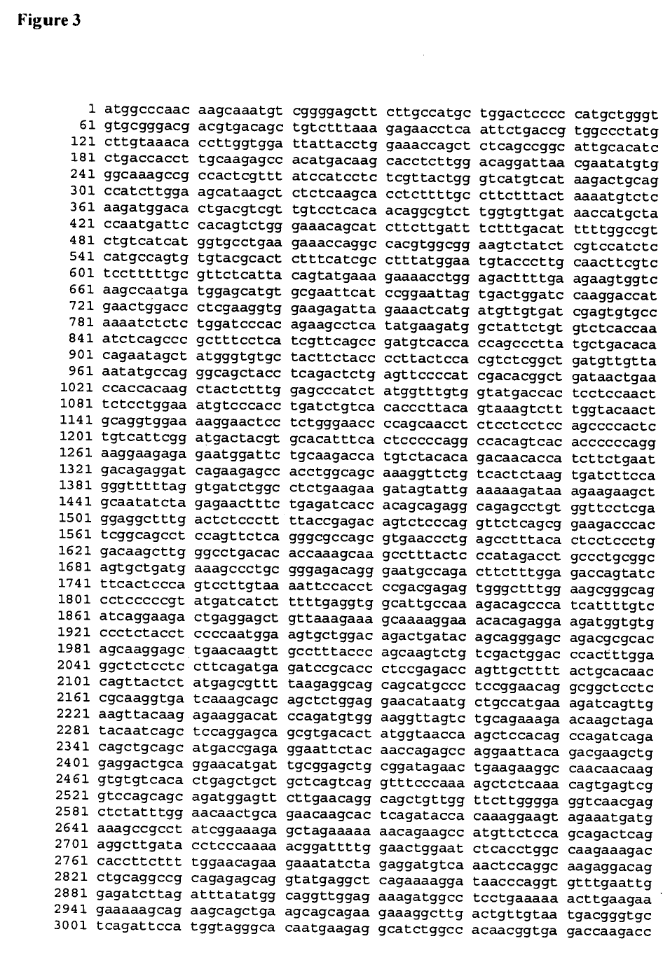

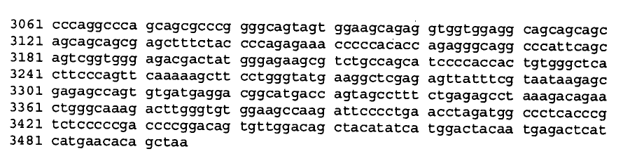

“TSC1 gene” refers to the TSC1 coding sequence open reading frame, as shown in FIG. 3 (SEQ ID NO:3), or any homologous sequence thereof preferably having at least 70% identity, more preferable 80% identity, even more preferably 90% or 95% identity. This encompasses any sequences derived from SEQ ID NO:3 which have undergone mutations, alterations or modifications as described herein.

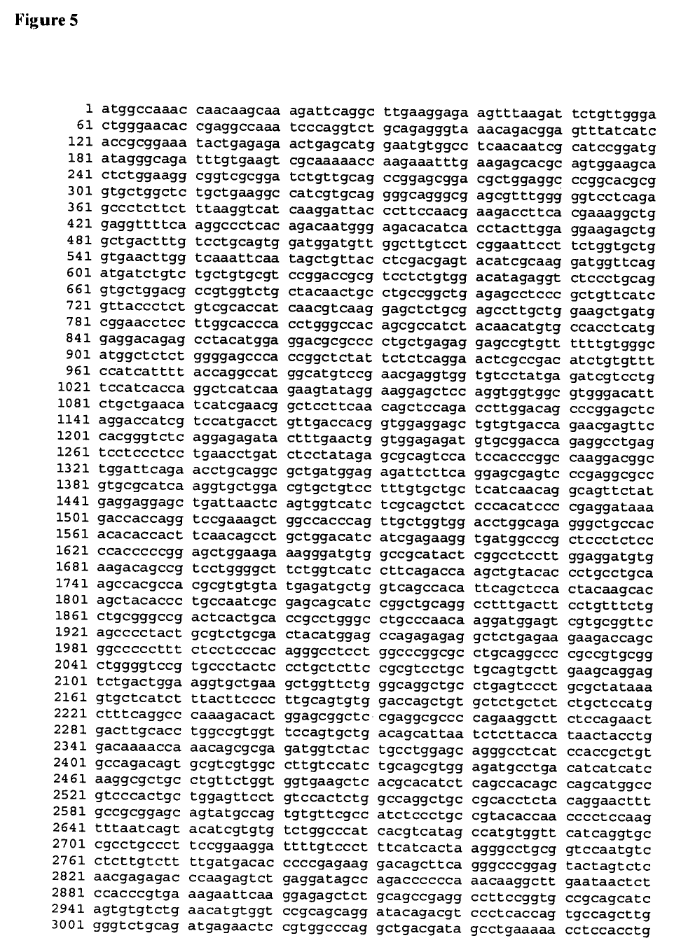

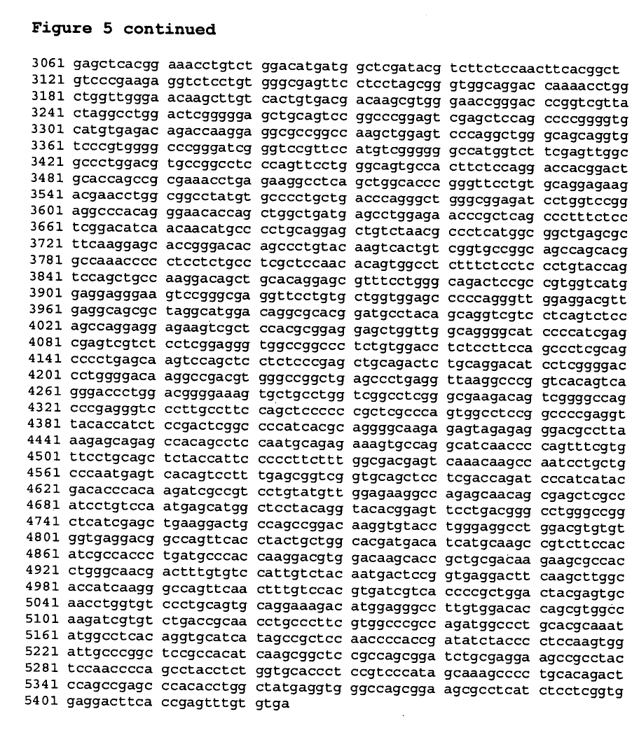

“TSC2 gene” refers to the TSC2 coding sequence open reading frame, as shown in FIG. 5 (SEQ ID NO:5), or any homologous sequence thereof preferably having at least 70% identity, more preferable 80% identity, even more preferably 90% or 95% identity. This encompasses any sequences derived from SEQ ID NO:5 which have undergone mutations, alterations or modifications as described herein.

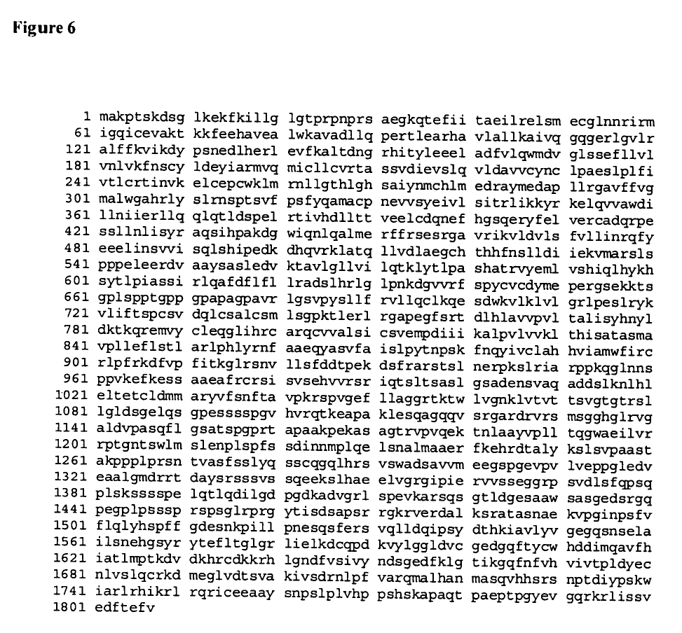

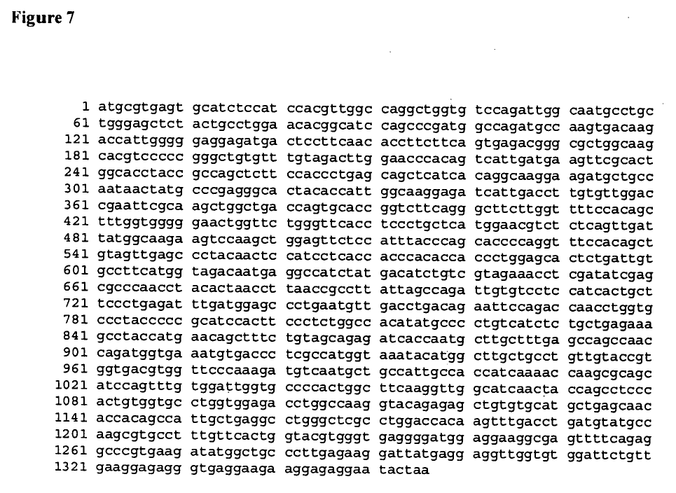

“Alpha-tubulin gene” refers to the alpha-tubulin coding sequence open reading frame, as shown in FIG. 7 (SEQ ID NO:7), or any homologous sequence thereof preferably having at least 70% identity, more preferable 80% identity, even more preferably 90% or 95% identity. This encompasses any sequences derived from SEQ ID NO:7 which have undergone mutations, alterations or modifications as described herein.

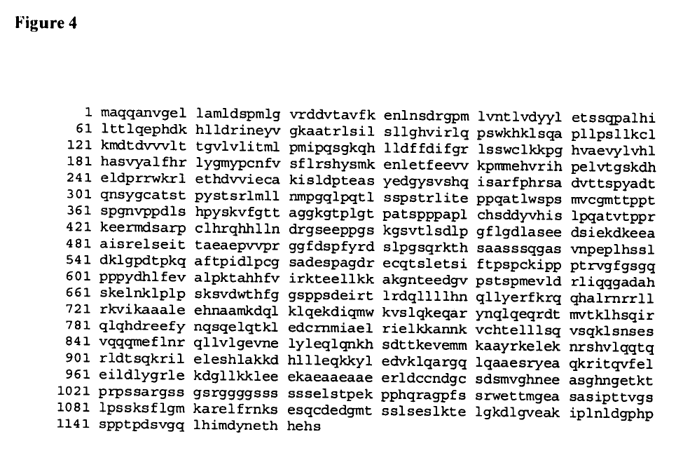

“TSC1 polypeptide” refers to the polypeptide of the TSC1 gene, also known as hamartin, derived from any organism, optionally man, splice variants and fragments thereof retaining biological activity, and homologs thereof, preferably having at least 70%, more preferably at least 80%, even more preferably at least 90% or 95% homology thereto. In addition, this term is understood to encompass polypeptides resulting from minor alterations in the TSC1 coding sequence, such as, inter alia, point mutations, substitutions, deletions and insertions which may cause a difference in a few amino acids between the resultant polypeptide and the naturally occurring TSC1. Polypeptides encoded by nucleic acid sequences which bind to the TSC1 coding sequence or genomic sequence under conditions of highly stringent hybridization, which are well-known in the art (for example Ausubel et al., Current Protocols in Molecular Biology, John Wiley and Sons, Baltimore, Md. (1988), updated in 1995 and 1998), are also encompassed by this term. Chemically modified TSC1 or fragments of TSC1, which may or may not be chemically modified, are also included in the term, so long as they are still capable of binding RTP801L. TSC1 preferably has or comprises an amino acid sequence according to SEQ. ID. NO. 4. It is acknowledged that there might be differences in the amino acid sequence among various tissues of an organism and among different organisms of one species or among different species to which the nucleic acid according to the present invention can be applied in various embodiments of the present invention. However, based on the technical teaching provided herein, the respective sequence can be taken into consideration accordingly when designing any of the nucleic acids according to the present invention. Particular fragments of TSC1 include amino acids 1-50, 51-100,101-150, 151-200 and 201-250, 251-300, 301-350, 351-400, 401-450, 451-500, 501-550, 551-600, 601-650, 651-700, 701-750, 751-800, 801-850, 851-900, 901-950, 951-1000, 1001-1050, 1051-1100 and 1101-1164 of the sequence shown in FIG. 4. Further particular fragments of TSC1 include amino acids 25-74, 75-124, 125-174, 175-224, 225-274, 275-324, 325-374, 375-424, 425-474, 475-524, 525-574, 575-624, 625-674, 675-724, 725-774, 775-824, 825-874, 875-924, 925-974, 975-1024, 1025-1074, 1075-1124 and 1125-1164 of the sequence shown in FIG. 4.

“TSC2 polypeptide” refers to the polypeptide of the TSC2 gene, also known as tuberin, derived from any organism, optionally man, splice variants and fragments thereof retaining biological activity, and homologs thereof, preferably having at least 70%, more preferably at least 80%, even more preferably at least 90% or 95% homology thereto. In addition, this term is understood to encompass polypeptides resulting from minor alterations in the TSC2 coding sequence, such as, inter alia, point mutations, substitutions, deletions and insertions which may cause a difference in a few amino acids between the resultant polypeptide and the naturally occurring TSC2. Polypeptides encoded by nucleic acid sequences which bind to the TSC2 coding sequence or genomic sequence under conditions of highly stringent hybridization, which are well-known in the art (for example Ausubel et al., Current Protocols in Molecular Biology, John Wiley and Sons, Baltimore, Maryland (1988), updated in 1995 and 1998), are also encompassed by this term. Chemically modified TSC2 or fragments of TSC2, which may or may not be chemically modified, are also included in the term, so long as they are still capable of binding RTP801L. TSC2 preferably has or comprises an amino acid sequence according to SEQ. ID. NO. 6. It is acknowledged that there might be differences in the amino acid sequence among various tissues of an organism and among different organisms of one species or among different species to which the nucleic acid according to the present invention can be applied in various embodiments of the present invention. However, based on the technical teaching provided herein, the respective sequence can be taken into consideration accordingly when designing any of the nucleic acids according to the present invention. Particular fragments of TSC2 include amino acids 1-50, 51-100,101-150, 151-200 and 201-250, 251-300, 301-350, 351-400, 401-450, 451-500, 501-550, 551-600, 601-650, 651-700, 701-750, 751-800, 801-850, 851-900, 901-950, 951-1000, 1001-1050, 1051-1100 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750 and 1751-1807 of the sequence shown in FIG. 6. Further particular fragments of TSC2 include amino acids 25-74, 75-124, 125-174, 175-224, 225-274, 275-324, 325-374, 375-424, 425-474, 475-524, 525-574, 575-624, 625-674, 675-724, 725-774, 775-824, 825-874, 875-924, 925-974, 975-1024, 1025-1074, 1075-1124, 1125-1174, 1175-1224, 1225-1274, 1275-1324, 1325-1374, 1375-1424, 1425-1474, 1475-1524, 1525-1574, 1575-1624, 1625-1674, 1675-1724, 1725-1774 and 1775-1807 of the sequence shown in FIG. 6.

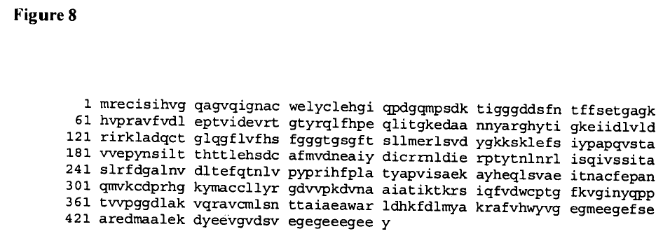

“Alpha-tubulin polypeptide” refers to the polypeptide of the alpha-tubulin gene derived from any organism, optionally man, splice variants and fragments thereof retaining biological activity, and homologs thereof, preferably having at least 70%, more preferably at least 80%, even more preferably at least 90% or 95% homology thereto. In addition, this term is understood to encompass polypeptides resulting from minor alterations in the alpha-tubulin coding sequence, such as, inter alia, point mutations, substitutions, deletions and insertions which may cause a difference in a few amino acids between the resultant polypeptide and the naturally occurring alpha-tubulin. Polypeptides encoded by nucleic acid sequences which bind to the alpha-tubulin coding sequence or genomic sequence under conditions of highly stringent hybridization, which are well-known in the art (for example Ausubel et al., Current Protocols in Molecular Biology, John Wiley and Sons, Baltimore, Md. (1988), updated in 1995 and 1998), are also encompassed by this term. Chemically modified alpha-tubulin or fragments of alpha-tubulin, which may or may not be chemically modified, are also included in the term, so long as they are still capable of binding RTP801L. alpha-tubulin preferably has or comprises an amino acid sequence according to SEQ. ID. NO. 8. It is acknowledged that there might be differences in the amino acid sequence among various tissues of an organism and among different organisms of one species or among different species to which the nucleic acid according to the present invention can be applied in various embodiments of the present invention. However, based on the technical teaching provided herein, the respective sequence can be taken into consideration accordingly when designing any of the nucleic acids according to the present invention. Particular fragments of alpha-tubulin include amino acids 1-50, 51-100,101-150, 151-200, 201-250, 251-300, 301-350, 351-400 and 401-451 of the sequence shown in FIG. 8. Further particular fragments of alpha-tubulin include amino acids 25-74, 75-124, 125-174, 175-224, 225-274, 275-324, 325-374, 375-424 and 425-451 of the sequence shown in FIG. 8.

RT801L, also referred to as “REDD2”, is related to RTP801. RTP801L is homologous to RTP801, and reacts in a similar manner to oxidative stress; thus, RTP801L possesses some similar functions with RTP801.

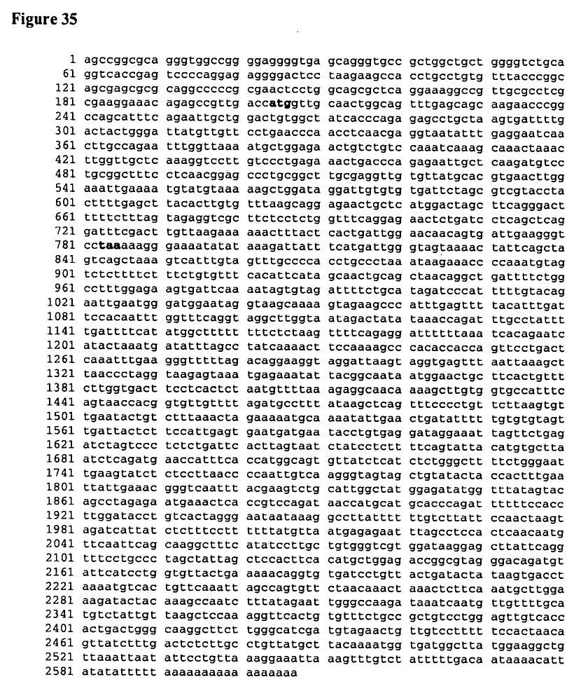

“RTP801L gene” refers to the RTP801L coding sequence open reading frame, as shown in FIG. 35 (SEQ ID NO:9), or any homologous sequence thereof preferably having at least 70% identity (see comment below), more preferable 80% identity, even more preferably 90% or 95% identity. This encompasses any sequences derived from SEQ ID NO:9 which have undergone mutations, alterations or modifications as described herein. Thus, in a preferred embodiment RTP801L is encoded by a nucleic acid sequence according to SEQ. ID. NO. 9. It is also within the present invention that the nucleic acids according to the resent invention are only complementary and identical, respectively, to a part of the nucleic acid coding for RTP801L as, preferably, the first stretch and first strand is typically shorter than the nucleic acid according to the present invention. It is also to be acknowledged that based on the amino acid sequence of RTP801L any nucleic acid sequence coding for such amino acid sequence can be perceived by the one skilled in the art based on the genetic code. However, due to the assumed mode of action of the nucleic acids according to the present invention, it is most preferred that the nucleic acid coding for RTP801L, preferably the mRNA thereof, is the one present in the organism, tissue and/or cell, respectively, where the expression of RTP801L is to be reduced.

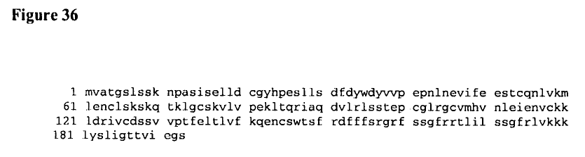

“RTP801L polypeptide” refers to the polypeptide of the RTP801L gene, and is understood to include, for the purposes of the instant invention, the terms “RTP777”, “REDD2”, and “SMHS1”, derived from any organism, optionally man, splice variants and fragments thereof retaining biological activity, and homologs thereof, preferably having at least 70%, more preferably at least 80%, even more preferably at least 90% or 95% homology thereto. In addition, this term is understood to encompass polypeptides resulting from minor alterations in the RTP801L coding sequence, such as, inter alia, point mutations, substitutions, deletions and insertions which may cause a difference in a few amino acids between the resultant polypeptide and the naturally occurring RTP801L. Polypeptides encoded by nucleic acid sequences which bind to the RTP801L coding sequence or genomic sequence under conditions of highly stringent hybridization, which are well-known in the art (for example Ausubel et al., Current Protocols in Molecular Biology, John Wiley and Sons, Baltimore, Md. (1988), updated in 1995 and 1998), are also encompassed by this term. Chemically modified RTP801L or chemically modified fragments of RTP801L are also included in the term, so long as the biological activity is retained. RTP801L preferably has or comprises an amino acid sequence according to SEQ. ID. NO. 10. It is acknowledged that there might be differences in the amino acid sequence among various tissues of an organism and among different organisms of one species or among different species to which the nucleic acid according to the present invention can be applied in various embodiments of the present invention. However, based on the technical teaching provided herein, the respective sequence can be taken into consideration accordingly when designing any of the nucleic acids according to the present invention. Particular fragments of RTP801L include amino acids 1-50, 51-100,101-150 and 151-193 of the sequence shown in FIG. 36. Further particular fragments of RTP801L include amino acids 25-74, 75-124, 125-174 and 175-193 of the sequence shown in FIG. 36.

Without being bound by theory, RTP801L may be involved in fine-tuning of cell response to energy misbalance. As such, it is a target suitable for treatment of any disease where cells should be rescued from apoptosis due to stressful conditions (e.g. diseases accompanied by death of normal cells) or where cells, which are adapted to stressful conditions due to changes in RTP801L expression (e.g. cancer cells), should be killed. In the latter case, RTP801L may be viewed as a survival factor for cancer cells and its inhibitors may treat cancer as a monotherapy or as sensitising drugs in combination with chemotherapy or radiotherapy.

The inventors of the present invention have discovered that alpha-tubulin binds RTP801L, and thus, alpha-tubulin can be employed in screening systems aimed at identifying RTP801L modulators. Detection of the activity of RTP801L modulators can be accomplished by assaying for an RTP801L—alpha-tubulin complex, or by tubulin polymerization assays.

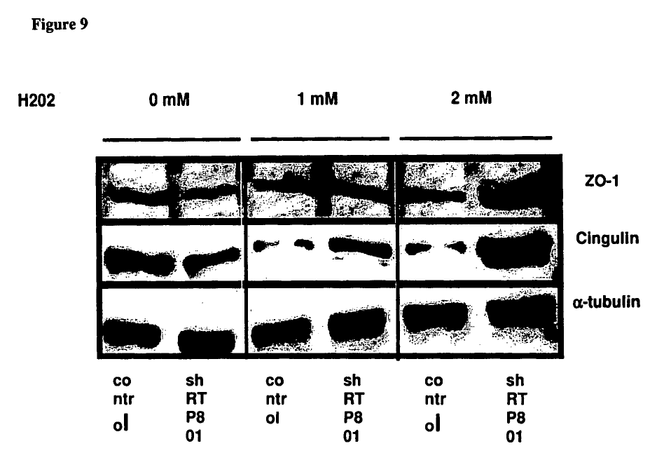

The inventors of the present invention have also discovered that inhibition of RTP801 expression results in increased amounts of the tight junction proteins cingulin and ZO-1 in H₂0₂-treated cells (see Example 3 and FIG. 9). Further, the inventors of the present invention have also discovered that RTP801 binds cyto-keratin9. Similar results are achieved with RTP801L. Said tight-junction proteins or cyto-keratin9 are used in all the methods of the present invention, as output indications in screening systems alone or in conjunction with other polypeptides disclosed herein. Further, additional tight junction proteins may also be used in the same capacity if desired.

Thus, in one embodiment the present invention comprises a process for determining whether a test compound modulates the activity of an RTP801L polypeptide comprising the following steps:
a) providing an RTP801L polypeptide and a second polypeptide selected from the group consisting of RTP801, RTP801L, TSC1, TSC2 and alpha-tubulin;
(b) treating or contacting the polypeptides of a) with the test compound;
(c) determining the amount of a complex comprising the RTP801L polypeptide and the second polypeptide; and
(d) comparing the amount of such complex determined in step c) with the amount determined for control polypeptides not treated or contacted with the test compound.
   and optionally wherein a difference in the amount determined in step c) with the amount determined for the control polypeptides indicates that the test compound modulates the activity of RTP801L.

As discussed above, the activity of the RTP801L polypeptide encompasses its ability to form a complex with one or more polypeptide, which is optionally selected from the group consisting of RTP801, RTP801L, TSC1, TSC2 and alpha-tubulin. The continuing activity exerted by the formation of such a complex may relate to the mTOR pathway and/or apoptosis, inter alia., but the complex formation in itself is defined as RTP801L activity, and a compound which disturbs or disrupts the formation of such a complex thereby modulates the activity of RTP801L. A compound which enhances the formation of such a complex also modulates the activity of RTP801L.

Additionally, the present invention further comprises the above process wherein one or both of the polypeptides are substantially purified, or wherein the RTP801L polypeptide is a form of RTP801L comprising a tag, or wherein the second polypeptide is a form of the second polypeptide comprising a tag, or wherein the RTP801L polypeptide is a form of RTP801L comprising a first tag and the second polypeptide is a form of the second polypeptide comprising a second tag. Further, one of the polypeptides may be attached to a solid support. Any of the polypeptides provided in the above process or any other processes of the present invention may be provided in a sample, and the subsequent steps of any of these processes performed on this sample.

The present invention additionally comprises a process for determining whether a test compound modulates the activity of an RTP801L polypeptide comprising the following steps:
(a) providing a cell which expresses
   (i) an RTP801L polypeptide and
   (ii) a second polypeptide selected from the group consisting of RTP801, RTP801L, TSC 1, TSC2 and alpha-tubulin;
(b) treating or contacting the cell of (a) with the test compound;
(c) determining the amount of a complex comprising the RTP801L polypeptide and the second polypeptide present in the cell; and
(d) comparing the amount of such complex determined in step c) with the amount determined in a control cell not treated or contacted with the test compound.
   and optionally wherein a difference in the amount determined in step c) with the amount determined in the control cell indicates that the test compound modulates the activity of RTP801L.

Additionally, a lysate may be prepared from the cell of step (b) and the detection of step (c) may be performed on the lysate. Further, a lysate may be prepared from the cell of step (a) and the treatment of step b) and detection of step (c) may be performed on the lysate.

In an additional embodiment, the present invention comprises a process for determining whether a test compound modulates the activity of RTP801L comprising the following steps:

a) providing a cell which expresses
(i) a form of RTP801L comprising a first tag; and
(ii) a form of a second polypeptide selected from the group consisting of RTP801, RTP801L, TSC1, TSC2 and alpha-tubulin, wherein the second polypeptide comprises a second tag;

(b) treating or contacting the cell of (a) with the test compound;

(c) determining the amount of a complex comprising the tagged form of RTP801L and the tagged form of the second polypeptide present in the cell; and

(d) comparing the amount of such complex determined in step c) with the amount determined in a control cell not treated or contacted with the test compound.

and optionally wherein a difference in the amount determined in step c) with the amount determined in the control sample indicates that the test compound modulates the activity of RTP801 L.

Additionally, a lysate may be prepared from the cell of step (b) and the detection of step (c) may be performed on the lysate. Further, a lysate may be prepared from the cell of step (a) and the treatment of step b) and detection of step (c) may be performed on the lysate.

Further, the first tag and the second tag may interact to produce a moiety, the amount of which can be determined. Exemplary moieties are discussed further below.

The present invention additionally provides a process for determining whether a test compound modulates the activity of an RTP801L polypeptide comprising the following steps:
a) providing an RTP801L polypeptide;
(b) treating or contacting the polypeptide of a) with the test compound;
(c) determining the amount of an RTP801L polypeptide complex; and
(d) comparing the amount of such complex determined in step c) with the amount determined for a control RTP801L polypeptide not treated or contacted with the test compound.
   and optionally wherein a difference in the amount determined in step c) with the amount determined for the control polypeptides indicates that the test compound modulates the activity of RTP801L.

The RTP801L polypeptide may be substantially purified; further, a portion of the RTP801L polypeptide may be a form of RTP801L comprising a tag. Additionally, a first portion of the RTP801L polypeptide may be a form of RTP801L comprising a first tag and the second portion of the RTP801L polypeptide may be a form of RTP801L comprising a second tag. Further, a portion of the RTP801L polypeptide may be attached to a solid support. Additionally, the complex formed may be a dimer.

Further provided is a process for obtaining a compound which modulates apoptosis in a cell comprising:

a) providing cells which express the human RTP801L polypeptide;

b) contacting the cells with a plurality of compounds;

c) determining which of the plurality of compounds modulates apoptosis in the cells; and

d) obtaining the compound determined to modulate apoptosis in step c).

The process may additionally comprise:

a) providing cells which express the human RTP801L polypeptide at a level such that about 50% of the cells undergo apoptosis in the presence of a known apoptosis-stimulating agent;

b) contacting the cells with the plurality of compounds;

c) treating the cells with an amount of the known apoptosis-stimulating agent so as to cause apoptosis in the cells;

d) determining which of the plurality of compounds modulates apoptosis in the cells; and

e) obtaining the compound determined to modulate apoptosis in step d).

An additionally embodiment comprises a process for obtaining a compound which modulates the activity of the RTP801L polypeptide comprising:

a) measuring the activity of the RTP801L polypeptide;

b) contacting the RTP801L polypeptide with a plurality of compounds;

c) determining which of the plurality of compounds modulates the activity of the RTP801L polypeptide; and

d) obtaining the compound determined to modulate the activity of the RTP801L polypeptide in step c).

Further provided is a process for obtaining a compound which modulates the activity of the RTP801L polypeptide comprising:
a) measuring the binding of the RTP801L polypeptide to a species with which the RTP801L polypeptide interacts;

b) contacting the RTP801L polypeptide with a plurality of compounds;

c) determining which of the plurality of compounds modulates the binding of the of the RTP801L polypeptide to the species; and

d) obtaining the compound determined to modulate the binding of the RTP801L polypeptide to the species in step c).

Additionally provided is a kit for obtaining a compound which modulates the biological activity of RTP801L comprising:

(a) RTP801L; and

(b) an interactor with which RTP801L interacts.

The interactor may be selected from the group consisting of an RTP801 polypeptide, a TSC1 polypeptide, a TSC2 polypeptide and an alpha-tubulin polypeptide.

In an additional embodiment, the present invention provides a process for identifying a compound which modulates the activity of RTP801L comprising the following steps:
a) providing a cell which expresses an RTP801L polypeptide and a second polypeptide selected from RTP801, RTP801L, TSC1, TSC2 and alpha-tubulin;
(b) treating the cell of (a) with a chemical compound;
(c) detecting the amount of a complex comprising RTP801L and the second polypeptide as compared to an untreated cell.

This process may be performed on cells or cell lysates, or alternatively in vitro using purified polypeptides instead of cells. The process would then comprise:
a) providing a purified RTP801L polypeptide
b) mixing the purified RTP801L polypeptide with a second purified polypeptide selected from RTP801, RTP801L, TSC1, TSC2 and alpha-tubulin;
(b) exposing the mixture of b) to a chemical compound;
(c) detecting the amount of a complex comprising RTP801L and the second polypeptide as compared to an unexposed sample.

The detection of polypeptides in any of the processes of the present invention may be performed using specific antibodies. Protein complexes may also be detected via gel electrophoresis (for example, under native conditions) or other methods known to those of skill in the art.

Additionally, as disclosed herein, the methods of the present invention may be performed using tagged polypeptides.

Thus, in another embodiment, the present invention provides a process for identifying a compound which modulates the activity of RTP801L comprising the following steps:

a) providing a cell which expresses RTP801L comprising, a first tag and which also expresses a second polypeptide selected from RTP801, RTP801L, TSC1, TSC2 and
alpha-tubulin, wherein the second polypeptide comprises a second tag;
(b) treating the cell of (a) with a chemical compound;
(c) detecting the amount of a complex comprising RTP801L and the second polypeptide as compared to a control.

Further provided is a process for identifying a compound which modulates the activity of RTP801L comprising the steps as above, wherein a lysate may be is prepared from the cell lo of step (b) and the detection of step (c) may be performed on the lysate. Further, a lysate may be prepared from the cell of step (a) and the treatment of step b) and detection of step (c) may be performed on the lysate.

In a particular embodiment, there is provided a process for identifying a compound which modulates the activity of RTP801L comprising the following steps:

a) providing a cell which expresses RTP801L comprising a first tag and which also expresses RTP801L comprising a second tag;

(b) treating the cell of (a) with a chemical compound;

(c) detecting the amount of an RTP801L homodimer as compared to a control cell.

Further provided is a process for identifying a compound which modulates the activity of RTP801L comprising the steps as above, wherein a lysate may be is prepared from the cell of step (b) and the detection of step (c) may be performed on the lysate. Further, a lysate may be prepared from the cell of step (a) and the treatment of step b) and detection of step (c) may be performed on the lysate.

Additionally provided is a process for identifying a compound which modulates the activity of RTP801L comprising the following steps:

a) providing purified RTP801L comprising a first tag;

b) providing purified RTP801L comprising a second tag;

(b) mixing a) and b) in vitro under binding conditions;

(c) detecting the amount of an RTP801L homodimer or oligomer as compared to a control sample.

Additionally, the present invention provides for a process for identifying a compound which modulates the activity of RTP801L comprising the following steps:
a) providing a cell which expresses RTP801L comprising a first tag and which also expresses a second polypeptide selected from RTP801, RTP801L, TSC1, TSC2 and alpha-tubulin, wherein the second polypeptide comprises a second tag, whereby the first and second tag interact in-vivo resulting in a detectable moiety;
b) treating the cells of step a) with a chemical compound;
c) detecting the amount of the detectable moiety in the cells or in a lysate of the cells as compared to a control.

Said detectable moiety may comprise, for example, a fluorescent molecule or protein, such as the split-YFP (BiFC) linker tagging system (Bracha-Drori et al, Plant J., 2004 Nov;40(3):419-27) or fluorescence achieved in a FRET or BRET (Issad T., et al., “The use of bioluminescence resonance energy transfer for the study of therapeutic targets: application to tyrosine kinase receptors” ert Opin Ther Targets. 2007 April;11(4):541-56; Koterba & Rowan, “Measuring ligand-dependent and ligand-independent interactions between nuclear receptors and associated proteins using Bioluminescence Resonance Energy Transfer (BRET)” Nucl Recept Signal. Jul. 26, 2006;4:e021; Prinz A., et al., “Application of bioluminescence resonance energy transfer (BRET) for biomolecular interaction studies” Chembiochem. 2006 Jul;7(7):1007-12) system, or a system based on an interaction detectable using, for example, western or protein blotting, such as an avidin-biotin interaction.

The control used in the processes of the present invention typically comprises an untreated cell, i.e., an identical cell which is not treated with a chemical. The control may additionally comprise a cell which does not express either TSC1, TSC2, RTP801 or alpha-tubulin (or cingulin, ZO-1 or cyto-keratin9), or a cell which expresses RTP801L but does not express TSC1, TSC2, RTP801 or alpha-tubulin (or cingulin, ZO-1 or cyto-keratin9), or a cell which cxpresses TSC1, TSC2, RTP801 or alpha-tubulin (or cingulin, ZO-1 or cyto-keratin9) but does not express RTP801L respectively. Preferably, said control cell expresses the necessary endogenous level of said polypeptides, in any of the combinations described, but does not over-express one or more of the polypeptides in question. Further, the control cell may comprise a cell essentially identical in its expression profile to the treatment cell, wherein the overexpressing polypeptides in the control cell do not comprise a tag.

According to the present invention, expression of RTP801L nucleic acid molecules and activity of RTP801L polypeptides are used in the screening of various compounds in order to obtain those which may be active in modulating the apoptotic process or the mTOR pathway, inter alia.

In a cell-based embodiment of this aspect of the invention, there is provided a process for obtaining a compound which modulates apoptosis in a cell comprising:

a) providing cells which express the human RTP801L polypeptide;

b) contacting said cells with said compound; and

c) determining the ability of said compound to modulate apoptosis in the cells.

The process may further comprise:

a) providing test cells and control cells which express the human RTP801L polypeptide at a level at which approximately 50% of the cells undergo apoptosis in the presence of an apoptosis-stimulating agent;

b) contacting said test cells with said compound;

c) treating said cells in conjunction with step (b) with an amount of apoptosis-stimulating agent capable of causing apoptosis in the control cell; and

d) determining the ability of said compound to modulate apoptosis in the test cell.

The process may further comprise:

a) providing a test cell which expresses the human RTP801L polypeptide and a control cell which does not express the human RTP801L polypeptide;

b) contacting said cells with said compound;

c) treating said cells in conjunction with step (b) with an amount of apoptosis-stimulating agent capable of causing apoptosis in the control cell but not in the test cell in the absence of said compound; and

d) determining the ability of said compound to promote apoptosis in the test cell.

Any of the above apoptosis-based methods may also be conducted on cells which overexpress or have reduced expression of a polypeptide selected from the group consisting of RTP801, TSC1, TSC2, alpha-tubulin, cingulin, ZO-1 or cytokeratin9.

In the processes of the invention, a preferred apoptosis-stimulafing agent may be a Fas activating agent such as a Fas ligand or an anti-Fas activating antibody or a chemotherapeutic drug such as those described above, or an analog of one of these chemotherapeutic drugs or a chemical analog or homolog thereof, or irradiation such as gamma irradiation. Additionally, the cells used in the above assays may be stimulated by treatment with cobalt, which causes the collapse of mitochondrial function in the cells and simulates some aspects of hypoxic and/or apoptotic states.

All of the screening methods described herein may be up-scaled to a larger scale format (including an industrial up-scaling) by methods known in the art. One up-scaling possibility involves transferring all the above methods to well plates comprising 96, 192, 384 or any other number of wells, which may serve in automated versions of the methods of the present invention. Up-scaling the methods of the present invention may involve performing them on a solid support, and possibly automating various steps of the methods. Appropriate automation procedures and solid supports are known to those of skill in the art. For example, a large-scale method according to the present invention may comprise the following steps:

(a) obtaining a solid support coated with purified RTP801L polypeptide;

(b) incubating the solid support with a lysate from cells which overexpress a tagged polypeptide selected from the group consisting of RTP801, RTP801L, TSC1, TSC2 and alpha-tubulin;

(c) washing the solid support;

(d) treating the solid support with a molecule such as a compound, chemical, siRNA or other potentially inhibitory molecule of any kind;

(e) washing the solid support; and

(f) assaying for the ability of the molecule of step (d) to disrupt the interaction between the tagged polypeptide of step (b) and RTP801L.

The purified polypeptide of step a and the tagged polypeptide of step b are interchangeable and thus, the methods may be performed with purified RTP801, RTP801L, TSC1, TSC2 or alpha-tubulin in step (a) and tagged RTP801L in step (b). Further, said method may be performed with any fragment of a relevant polypeptide, such as the particular fragments disclosed herein or any other biologically active fragment, i.e., a fragment that retains the relevant binding activity of the parent polypeptide.

A variety of tags for tagging polypeptides may be used with any of the methods of the present invention, such as fluorescent tags (fluorescent protein fusions, alexa dyes, cy dyes, FITC, etc.), biotin, amino acid tags (Myc, HA, 1A8, His) Flag, and GST, inter alia. The word “tag” is understood to include both cases where the mature polypeptide is bound to the tag by various chemical or biochemical means, and cases where the polypeptide is expressed as a fusion to the tag by biological means (expressed and purified from a bacterial system, or expressed directly as a fusion protein in mammalian systems).

It will be appreciated that, based on knowledge of the RTP801L polypeptide, it is possible to devise a non cell-based assay for screening for, i.e. obtaining compounds which modulate apoptosis through the human RTP801L polypeptide. An example of such a non cell-based assay is described below. Without being bound by theory, the anti-apoptotic effect of the RTP801L polypeptide may be due to the specific binding or interaction of part or all of the RTP801L polypeptide to a different species such as, without limitation, a factor, molecule, or specific binding substance, and this effect may be monitored by linking this specific binding or interaction to a signaling system. It is thus an aim of the present invention to identify compounds which, for example, modulate or disturb this specific interaction of the RTP801L polypeptide with such species.

Therefore, in a non cell-based embodiment there is provided a process for obtaining a compound which modulates apoptosis through the human RTP801L polypeptide comprising:

a) measuring activity of the human RTP801L polypeptide;

b) contacting said polypeptide with said compound; and

c) measuring the activity of said polypeptide as compared to a control.

For the purposes of this and other non-cell based assays, the activity of RTP801L may be in the modulation of apoptosis, as described herein; further, said activity may relate to the balance of reactive oxygen species in the sample being tested, or to the binding capacity of RTP801L to RTP801, RTP801L, TSC1, TSC2 or alpha-tubulin (or cingulin or ZO-1 or cyto-keratin9) in vitro.

Another non cell-based embodiment provides a process for obtaining a compound which modulates apoptosis through the human RTP801L polypeptide comprising:
a) measuring the binding of the human RTP801L polypeptide, or an active fragment thereof, to a species to which the human RTP801L polypeptide interacts specifically in vivo to produce an effect;

b) contacting said polypeptide or fragment with said compound; and

c) determining whether the activity of said polypeptide or fragment is affected by said compound.

The species may be RTP801, RTP801L, TSC1, TSC2 alpha-tubulin, cingulin, cyto-keratin9 or ZO-1, inter alia. Further, the effect may be an apoptosis modulation effect, an effect relating to energy metabolism or an effect on the mTOR pathway.

It is known that at times, fragments of polypeptides retain the essential biological properties of the parent, unfragmented polypeptide, and accordingly, a RTP801L DNA molecule useful in the methods of the present invention may also have a sequence encoding such fragments. Likewise, fragments of TSC1, TSC2 or alpha-tubulin may also be employed in the methods of the present invention. Preliminary results obtained by the inventors of the present invention indicate that the following fragments are useful in the screening systems of the present invention:

RTP801 N-fragment: a polypeptide comprising amino acids 1-88 of the RTP801 polypeptide, as presented in FIG. 2; this polypeptide serves as a control in TSC2 binding-based screening systems, and as a binding moiety in other screening systems.

RTP801 C-fragment: a polypeptide comprising amino acids 89-232 of the RTP801 polypeptide, as presented in FIG. 2; this polypeptide serves as a binding moiety in all the screening systems detailed herein, and may replace RTP801 in said systems, particularly those based on alpha-tubulin or TSC2 binding.

RTP801 N-C1 fragment: a polypeptide comprising amino acids 1-161 of the RTP801 polypeptide, as presented in FIG. 2.

RTP801 N-C2 fragment: a polypeptide comprising amino acids 1-195 of the RTP801 polypeptide, as presented in FIG. 2.

RTP801 C3 fragment: a polypeptide comprising amino acids 161-232 of the RTP801 polypeptide, as presented in FIG. 2.

RTP801 self association moiety: a polypeptide comprising amino acids 161-195 of the RTP801 polypeptide, as presented in FIG. 2.

RTP801L is homologous to RTP801 and the functional RTP801 fragments described above have parallel functional RTP801L fragments which are used in a similar capacity.

TSC2 N-fragment: a polypeptide comprising amino acids 1-935 of the TSC2 polypeptide, as presented in FIG. 6; this polypeptide can serve as control or replace TSC2 in all the TSC2 based assays of the present invention.

TSC2 C-fragment: a polypeptide comprising amino acids 853-1807 of the TSC2 polypeptide, as presented in FIG. 6; this polypeptide can serve as control or replace TSC2 in all the TSC2 based assays of the present invention.

Any of the methods of the present invention are practiced with the above fragments in lieu of their respective full-length polypeptides, as well as tagged fragments instead of tagged full-length polypeptides.

Said above fragments/polypeptides are in themselves novel and inventive and are considered per se a part of the present invention. Further details concerning the assays in which these fragments/polypeptides were used can be found in Examples 4-6.

An additional embodiment of the present invention concerns methods and processes for obtaining a species and/or chemical compound that modulates the biological activity of RTP801L. One aspect of this embodiment provides a process for obtaining a species and/or chemical compound that modulates the biological activity of RTP801L which comprises contacting a cell expressing RTP801L with a species and/or compound and determining the ability of the species and/or compound to modulate the biological activity of RTP801L of the cell as compared to a control. The cell being examined may be modified to express RTP801L, and without being bound by theory—apoptosis may be induced by the presence of RTP801L, or by neurotoxic stress, optionally caused by hydrogen peroxide, glutamate, dopamine, the Aβ protein or any known neurotoxin or neurotoxic treatment such as ischemia or hypoxia, or by a neurodegenerative disease such as stroke. In addition, this process may be used in order to prepare a pharmaceutical composition. The process then comprises admixing a species or compound obtained by the process recited above or a chemical analog or homolog thereof with a pharmaceutically acceptable carrier.

By cells being “modified to express” as used herein is meant that cells are modified by transfection, transduction, infection or any other known molecular biology method which will cause the cells to express the desired gene. Materials and protocols for carrying out such methods are evident to the skilled artisan.

Thus, an additional aspect of the screening embodiment provides a process of screening a plurality of species or compounds to obtain a species and/or compound that modulates the biological activity of RTP801L, which comprises:

(a) contacting cells expressing RTP801L with a plurality of species and/or chemical compounds;
(b) determining whether the biological activity of RTP801L is modulated in the presence of the species and/or compounds, as compared to a control; and if so
(c) separately determining whether the modulation of the biological activity of RTP801L is affected by each species and/or compound included in the plurality of species and/or compounds, so as to thereby identify the species and/or compound which modulates the biological activity of RTP801L.

The cells in the contacting step may be modified to express the RTP801L polypeptide, and—without being bound by theory—apoptosis may be induced spontaneously by RTP801L overexpression, or as a result of subjection of the cells to neurotoxic stress, optionally caused by hydrogen peroxide, glutamate, dopamine, the Aβ protein or any known neurotoxin or neurotoxic treatment such as ischemia or hypoxia, or by a neurodegenerative disease such as stroke. Further, the species may be a polypeptide such as, inter alia, RTP801, RTP801L, TSC1, TSC2, alpha-tubulin, cingulin, cyto-keratin9 or ZO-1, or any species which is known to have activity in the mTOR pathway. In addition, this process may be used in order to prepare a pharmaceutical composition. The process then comprises admixing a species or compound identified by the process recited above or a chemical analog or homolog thereof with a pharmaceutically acceptable carrier.

The process may additionally comprise modification of a species or compound found to modulate apoptosis by the above process to produce a compound with improved activity and admixing such compound with a pharmaceutically acceptable carrier. This additional act may be performed with a compound discovered by any of the processes which are disclosed in the screening embodiment of the present invention, so as to thereby obtain a pharmaceutical composition comprising a compound with improved activity.

Additionally, the screening embodiment of the present invention provides a non cell-based process for obtaining a species or compound which modulates the biological activity of RTP801L comprising:

(a) measuring the binding of RTP801L or the RTP801L gene to an interactor;

(b) contacting RTP801L or the RTP801L gene with said species or compound; and
(c) determining whether the binding of RTP801L or the RTP801L gene to said interactor is affected by said species or compound.

Said in-vitro system may be subjected to apoptotic conditions, which can be induced—without being bound by theory—by causing neurotoxic stress, as a result of treatment with, inter alia, hydrogen peroxide, glutamate, dopamine, the Aβ protein or any known neurotoxin. Further, said interactor may be RTP801, RTP801L, TSC1, TSC2, alpha-tubulin, cingulin, cyto-keratin9 or ZO-1, or any other interactor known to have activity in the mTOR pathway. In addition, this process may be used in order to prepare a pharmaceutical composition. The process then comprises admixing a species or compound identified by the process recited above or a chemical analog or homolog thereof with a pharmaceutically acceptable carrier.

Another aspect of the screening embodiment provided by the present invention concerns a kit for obtaining a species or compound which modulates the biological activity of RTP801L or the RTP801L gene in a cell comprising:
(a) RTP801L or the RTP801L gene; and
(b) an interactor with which RTP801L or the RTP801L gene interacts
(c) means for measuring the interaction of RTP801L or the RTP801L gene with the interactor; and
(d) means of determining whether the binding of RTP801L or the RTP801L gene to the interactor is affected by said species or compound.

The interactor in question may be RTP801, RTP801L, TSC1, TSC2, alpha-tubulin, cingulin, ZO-1 or cyto-keratin9; the interactor may also be a microtubule comprising or imicrotubule associated protein.

Means of measuring interactions between molecules and determining the strength, affinity, avidity and other parameters of the interaction are well known in the art (see, for example, Lubert Stryer, Biochemistry, W H Freeman & Co.; 5th edition (April 2002); and “Comprehensive Medicinal Chemistry”, by various authors and editors, published by Pergamon Press).

Interaction between RTP801L and TSC1 or TSC2 can be measured by assessing the activity of the mTOR pathway.

The activity and/or status of the mTOR pathway can be assessed, inter alia, by measuring Rheb activity; activity or phosphorylation state of S6K and/or eEF2K and/or 4E-BP1; TSC2 phosphorylation and HIF accumulation. For further information see: Jozwiak J, Jozwiak S, Grzela T, Lazarczyk M: Positive and negative regulation of TSC2 activity and its effects on downstream effectors of the mTOR pathway. Neuromolecular Med. 2005;7(4):287-96.; Brugarolas J, Lei K, Hurley R L, Manning B D, Reiling J H, Hafen E, Witters L A, Ellisen L W, Kaelin W G Jr.: Regulation of mTOR function in response to hypoxia by REDD1 and the TSC1/TSC2 tumor suppressor complex. Genes Dev. Dec. 1, 2004;18(23):2893-904.; Sofer A, Lei K, Johannessen C M, Ellisen L W.: Regulation of mTOR and cell growth in response to energy stress by REDD1. Mol Cell Biol. 2005 July;25(14):5834-45.; Corradetti M N, Inoki K, Guan K L: The stress-inducted proteins RTP801 and RTP801L are negative regulators of the mammalian target of rapamycin pathway. J Biol Chem. Mar. 18, 2005;280(11):9769-72.

Screening Systems

The RTP801L gene or polypeptide may be used in a screening assay for identifying and isolating compounds which modulate its activity such as the methods of screening for compounds which modulate RTP801L activity as disclosed herein. Compounds which modulate RTP801L activity typically also modulate neurotoxic stress or neurodegenerative diseases, and can thus be useful in the preparation of pharmaceutical compositions aimed at treating such conditions. The compounds to be screened comprise inter atia substances such as small chemical molecules, antibodies, antisense oligonucleotides, antisense DNA or RNA molecules, polypeptides and dominant negatives, and expression vectors. Many types of screening assays are known to those of ordinary skill in the art. The specific assay which is chosen depends to a great extent on the activity of the candidate gene or the polypeptide expressed thereby. Thus, if it is known that the expression product of a candidate gene has enzymatic activity, then an assay which is based on inhibition (or stimulation) of the enzymatic activity can be used. If the candidate polypeptide is known to bind to a ligand or other interactor, then the assay can be based on the inhibition of such binding or interaction. When the candidate gene is a known gene, then many of its properties can also be known, and these can be used to determine the best screening assay. If the candidate gene is novel, then some analysis and/or experimentation is appropriate in order to determine the best assay to be used to find inhibitors of the activity of that candidate gene. The analysis can involve a sequence analysis to find domains in the sequence which shed light on its activity.

As is well known in the art, the screening assays can be cell-based or non-cell-based. The cell-based assay is performed using eukaryotic cells such as HeLa cells, and such cell-based systems are particularly relevant in order to directly measure the activity of candidate genes which are anti-apoptotic functional genes, i.e., expression of the gene prevents apoptosis or otherwise prevents cell death in target cells. One way of running such a cell-based assay uses tetracycline-inducible (Tet-inducible) gene expression. Tet-inducible gene expression is well known in the art; see for example, Hofmann et al, 1996, Proc Natl Acad Sci 93(11):5185-5190.

Tet-inducible retroviruses have been designed incorporating the Self-inactivating (SIN) feature of a 3′ Ltr enhancer/promoter retroviral deletion mutant. Expression of this vector in cells is virtually undetectable in the presence of tetracycline or other active analogs. However, in the absence of Tet, expression is turned on to maximum within 48 hours after induction, with uniform increased expression of the whole population of cells that harbor the inducible retrovirus, thus indicating that expression is regulated uniformly within the infected cell population.

If the gene product of the candidate gene phosphorylates with a specific target protein, a specific reporter gene construct can be designed such that phosphorylation of this reporter gene product causes its activation, which can be followed by; a color reaction. The candidate gene can be specifically induced, using the Tet-inducible system discussed above, and a comparison of induced versus non-induced genes provides a measure of reporter gene activation.

In a similar indirect assay, a reporter system can be designed that responds to changes in protein-protein interaction of the candidate protein. If the reporter responds to actual interaction with the candidate protein, a color reaction occurs.

One can also measure inhibition or stimulation (referred to herein collectively as “modulation”) of e.g., reporter gene activity, by modulation of its expression levels via the specific candidate promoter or other regulatory elements. A specific promoter or regulatory element controlling the activity of a candidate gene is defined by methods well known in the art. A reporter gene is constructed which is controlled by the specific candidate gene promoter or regulatory elements. The DNA containing the specific promoter or regulatory agent is actually linked to the gene encoding the reporter. Reporter activity depends on specific activation of the promoter or regulatory element. Thus, inhibition or stimulation of the reporter is a direct assay of stimulafion/inhibition of the reporter gene; see, for example, Komarov et al (1999), Science vol 285,1733-7 and Storz et al (1999) Analytical Biochemistry, 276, 97-104.

Various non-cell-based screening assays are also well within the skill of those of ordinary skill in the art. For example, if enzymatic activity is to be measured, such as if the candidate protein has a kinase activity, the target protein can be defined and specific phosphorylation of the target can be followed. The assay can involve either inhibition of target phosphorylation or stimulation of target phosphorylation, both types of assay being well known in the art; for example see Mohney et al (1998) J.Neuroscience 18, 5285 and Tang et al (1997) J Clin. Invest. 100, 1180 for measurement of kinase activity. Additionally, there is a possibility that RTP801L interacts with an enzyme and regulates its enzymatic activity through protein-protein interaction.

One can also measure in vitro interaction of a candidate polypeptide with interactors. In this screen, the candidate polypeptide is immobilized on beads. An interactor, such as a receptor ligand, is radioactively labeled and added. When it binds to the candidate polypeptide on the bead, the amount of radioactivity carried on the beads (due to interaction with the candidate polypeptide) can be measured. The assay indicates inhibition of the interaction by measuring the amount of radioactivity on the bead.

Any of the screening assays, according to the present invention, can include a step of identifying the chemical compound (as described above) or other species which tests positive in the assay and can also include the further step of producing as a medicament that which has been so identified. It is considered that medicaments comprising such compounds, or chemical analogs or homologs thereof, are part of the present invention. The use of any such compounds identified for inhibition or stimulation of apoptosis is also considered to be part of the present invention.

Examples of viability assays that can be used with this bioassay include Annexin V stain (for apoptosis), and alamar blue or neutral red stains (for life/death).

An additional embodiment of the present invention concerns inhibition of the RTP801L gene or polypeptide for the treatment of eye diseases, respiratory disorders, microvascular disorders, hearing disorders and ischemic conditions, inter alia.

In addition to the above and without being bound by theory, the inventors of the present invention have found that RTP801L is involved in various disease states including microvascular disorders, eye diseases, respiratory disorders, hearing disorders, pressure sores, ischemic conditions and spinal cord injury and disease, and it would be beneficial to inhibit RTP801L in order to treat any of said diseases or disorders. Methods for identifying compounds and molecules that inhibit RTP801L are discussed herein at length, and any of said molecules and/or compositions may be beneficially employed in the treatment of a patient suffering from any of said conditions. Additionally, the molecules identified according to the methods of the present invention may potentially be used to treat patients suffering from diseases relating to abnormal function of the mTOR pathway, as well as diseases relating to abnormal TSC1 or TSC2 function such as, inter alia, tubular sclerosis.

The molecules identified according to the methods of the present invention and pharmaceutical compositions comprising them can have application in the treatment of any disease in which neuronal degeneration or damage is involved or implicated, such as, inter alia—the following conditions: hypertension, hypertensive cerebral vascular disease, a constriction or obstruction of a blood vessel—as occurs in the case of a thrombus or embolus, angioma, blood dyscrasias, any form of compromised cardiac function including cardiac arrest or failure, systemic hypotension,; and diseases such as stroke, Parkinson's disease, epilepsy, depression, ALS, Alzheimer's disease, Huntington's disease and any other disease-induced dementia (such as HIV induced dementia for example). These conditions are also referred to herein as “neurodegenerative diseases”. Trauma to the central nervous system, such as rupture of aneurysm, cardiac arrest, cardiogenic shock, septic shock, spinal cord trauma, head trauma, traumatic brain injury (TBI), seizure, bleeding from a tumor, etc., are also referred to herein as “injury to the central nervous system” and may also be treated using the compounds and compositions of the present invention.

The term “polynucleotide” refers to any molecule composed of DNA nucleotides, RNA nucleotides or a combination of both types, i.e. that comprises two or more of the bases guanidine, cytosine, thymidine, adenine, uracil or inosine, inter alia. A polynucleotide may include natural nucleotides, chemically modified nucleotides and synthetic nucleotides, or chemical analogs thereof. The term includes “oligonucleotides” and encompasses “nucleic acids”.

The term “amino acid” refers to a molecule which consists of any one of the 20 naturally occurring amino acids, amino acids which have been chemically modified (see below), or synthetic amino acids.

The term “polypeptide” refers to a molecule composed of two or more amino acids residues. The term includes peptides, polypeptides, proteins and peptidomimetics.

A “peptidomimetic” is a compound containing non-peptidic structural elements that is capable of mimicking the biological action(s) of a natural parent peptide. Some of the classical peptide characteristics such as enzymatically scissille peptidic bonds are normally not present in a peptidomimetic. Peptidomimetics may be used in the screening systems of the present invention.

By the term “dominant negative peptide” is meant a polypeptide encoded by a cDNA tragment that encodes for a part of a protein (see Herskowitz I.: Functional inactivation of genes by dominant negative mutations. Nature. Sep. 17-23, 1987;329(6136):219-22. Review; Roninson IB et al., Genetic suppressor elements: new tools for molecular oncology—thirteenth Cornelius P. Rhoads Memorial Award Lecture. Cancer Res. Sep. 15, 1995;55(18):4023). This peptide can have a different function from the protein from which it was derived. It can interact with the full protein and inhibit its activity or it can interact with other proteins and inhibit their activity in response to the full-length (parent) protein. Dominant negative means that the peptide is able to overcome the natural parent protein and inhibit its activity to give the cell a different characteristic, such as resistance or sensitization to death or any cellular phenotype of interest. For therapeutic intervention the peptide itself may be delivered as the active ingredient of a pharmaceutical composition, or the cDNA can be delivered to the cell utilizing known methods. Dominant negative peptides may be used in the screening systems of the present invention.

Preparation of Peptides and Polypeptides

Polypeptides may be produced via several methods, for example:

1) Synthetically:

Synthetic polypeptides can be made using a commercially available machine, using the known sequence of the desired protein or a portion thereof.

2) Recombinant Methods:

A preferred method of making the desired polypeptides of fragments thereof is to clone a polynucleotide comprising the cDNA of the desired gene into an expression vector and culture the cell harboring the vector so as to express the encoded polypeptide, and then purify the resulting polypeptide, all performed using methods known in the art as described in, for example, Marshak et al., “Strategies for Protein Purification and Characterization. A laboratory course manual.” CSHL Press (1996). (in addition, see Bibl Haematol. 1965,23.1165-74 Appl Microbiol. 1967 July;15(4):851-6; Can J Biochem. 1968 May;46(5):441-4; Biochemistry. 1968 July; 7(7):2574-80; Arch Biochem Biophys. Sep. 10, 1968;126(3):746-72; Biochem Biophys Res Commun. Feb. 20, 1970;38(4):825-30).).

The expression vector can include a promoter for controlling transcription of the heterologous material and can be either a constitutive or inducible promoter to allow selective transcription. Enhancers that can be required to obtain necessary transcription levels can optionally be included. The expression vehicle can also include a selection gene.

Vectors can be introduced into cells or tissues by any one of a variety of methods known within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al. (1986).

3) Purification from Natural Sources:

A desired polypeptide, or naturally occurring fragments thereof, can be purified from natural sources (such as tissues) using many methods known to one of ordinary skill in the art, such as for example: immuno-precipitation with an appropriate antibody, or matrix-bound affinity chromatography with any molecule known to bind the desired protein. Protein purification is practiced as is known in the art as described in, for example, Marshak et al., “Strategies for Protein Purification and Characterization. A laboratory course manual.” CSHL Press (1996).

“Apoptosis” refers to a physiological type of cell death which results from activation of some cellular mechanisms, i.e. death that is controlled by the machinery of the cell. Apoptosis may, for example, be the result of activation of the cell machinery by an external trigger, e.g. a cytokine or anti-FAS antibody, which leads to cell death or by an internal signal. The term “programmed cell death” may also be used interchangeably with “apoptosis”.

“Apoptosis-related disease” refers to a disease the etiology of which is related either wholly or partially to the process of apoptosis. The disease may be caused either by a malfunction of the apoptotic process (such as in cancer or an autoimmune disease) or by overactivity of the apoptotic process (such as in certain neurodegenerative diseases). Many diseases in which RTP801L is involved are apoptosis-related diseases. For example, apoptosis is a significant mechanism in dry AMD, whereby slow atrophy of photoreceptor and pigment epithelium cells, primarily in the central (macular) region of retina takes place. Neuroretinal apoptosis is also a significant mechanism in diabetic retinopathy.

An “inhibitor” is a compound which is capable of inhibiting the activity of a gene or the product of such gene to an extent sufficient to achieve a desired biological or physiological effect. An “RTP801L inhibitor” is a compound which is capable of inhibiting the activity of the RTP801L gene or RTP801L gene product, particularly the human RTP801L gene or gene product. Such inhibitors include substances that affect the transcription or translation of the gene as well as substances that affect the activity of the gene product. An RTP801L inhibitor may also be an inhibitor of the RTP801L promoter. Examples of such inhibitors may include, inter alia: polynucleotides such as AS fragments, siRNA, or vectors comprising them; polypeptides such as dominant negatives, antibodies, and enzymes; catalytic RNAs such as ribozymes; and chemical molecules with a low molecular weight e.g. a molecular weight below 2000 daltons. Specific RTP801L inhibitors are given below.

“Expression vector” refers to a vector that has the ability to incorporate and express heterologous DNA fragments in a foreign cell. Many prokaryotic and eukaryotic expression vectors are known and/or commercially available. Selection of appropriate expression vectors is within the knowledge of those having skill in the art.

The term “antibody” refers to IgG, IgM, IgD, IgA, and IgE antibody, inter alia. The definition includes polyclonal antibodies or monoclonal antibodies. This term refers to whole antibodies or fragments of antibodies comprising an antigen-binding domain, e.g. antibodies without the Fc portion, single chain antibodies, miniantibodies, fragments consisting of essentially only the variable, antigen-binding domain of the antibody, etc. The term “antibody” may also refer to antibodies against polynucleotide sequences obtained by cDNA vaccination. The term also encompasses antibody fragments which retain the ability to selectively bind with their antigen or receptor and are exemplified as follows, inter alia:
(1) Fab, the fragment which contains a monovalent antigen-binding fragment of an antibody molecule which can be produced by digestion of whole antibody with the enzyme papain to yield a light chain and a portion of the heavy chain;
(2) (Fab′)₂, the fragment of the antibody that can be obtained by treating whole antibody with the enzyme pepsin without subsequent reduction;
(Fab′₂) is a dimer of two Fab fragments held together by two disulfide bonds;
(3) Fv, defined as a genetically engineered fragment containing the variable region of the light chain and the variable region of the heavy chain expressed as two chains; and
(4) Single chain antibody (SCA), defined as a genetically engineered molecule containing the variable region of the light chain and the variable region of the heavy chain linked by a suitable polypeptide linker as a genetically fused single chain molecule.

By the term “epitope” as used in this invention is meant an antigenic determinant on an antigen to which the antibody binds. Epitopic determinants usually consist of chemically active surface groupings of molecules such as amino acids or sugar side chains and usually have specific three-dimensional structural characteristics, as well as specific charge characteristics.

Preparation of Antibodies

Antibodies which bind to a desired polypeptide or a fragment derived therefrom may be prepared using an intact polypeptide or fragments containing smaller polypeptides as the immunizing antigen. For example, it may be desirable to produce antibodies that specifically bind to the N- or C-terminal or any other suitable domains of the desired polypeptide. The polypeptide used to immunize an animal can be derived from translated cDNA or chemical synthesis and can be conjugated to a carrier protein, if desired. Such commonly used carriers which are chemically coupled to the polypeptide include keyhole limpet hemocyanin (KLH), thyroglobulin, bovine serum albumin (BSA) and tetanus toxoid. The coupled polypeptide is then used to immunize the animal.

If desired, polyclonal or monoclonal antibodies can be further purified, for example by binding to and elution from a matrix to which the polypeptide or a peptide to which the antibodies were raised is bound. Those skilled in the art know various techniques common in immunology for purification and/or concentration of polyclonal as well as monoclonal antibodies (Coligan et al, Unit 9, Current Protocols in Immunology, Wiley Interscience, 1994).

Methods for making antibodies of all types, including fragments, are known in the art (See for example, Harlow and Lane, Antibodies: A Laboratory Manual, Cold Spring Harbor Laboratory, New York (1988)). Methods of immunization, including all necessary steps of preparing the immunogen in a suitable adjuvant, determining antibody binding, isolation of antibodies, methods for obtaining monoclonal antibodies, and humanization of monoclonal antibodies are all known to the skilled artisan

The antibodies may be humanized antibodies or human antibodies. Antibodies can be humanized using a variety of techniques known in the art including CDR-grafting (EP239,400: PCT publication WO.91/09967; U.S. Pat. Nos. 5,225,539; 5,530,101; and 5,585,089, veneering or resurfacing (EP 592,106; EP 519,596; Padlan, Molecular Immunology 28(4/5):489-498 (1991); Studnicka et al., Protein Engineering 7(6):805-814 (1994); Roguska et al., PNAS 91:969-973 (1994)), and chain shuffling (U.S. Pat. No. 5,565,332).

The monoclonal antibodies as defined include antibodies derived from one species (such as murine, rabbit, goat, rat, human, etc.) as well as antibodies derived from two (or more) species, such as chimeric and humanized antibodies.

Completely human antibodies are particularly desirable for therapeutic treatment of human patients. Human antibodies can be made by a variety of methods known in the art including phage display methods using antibody libraries derived from human immunoglobulin sequences. See also U.S. Pat. Nos. 4,444,887 and 4,716,111; and PCT publications WO 98/46645, WO 98/50433, WO 98/24893, WO 98/16654, WO 96/34096, WO 96/33735, and WO 91/10741, each of which is incorporated herein by reference in its entirety.

Additional information regarding all types of antibodies, including humanized antibodies, human antibodies and antibody fragments can be found in WO 01/05998, which is incorporated herein by reference in its entirety.

Neutralizing antibodies can be prepared by the methods discussed above, possibly with an additional step of screening for neutralizing activity by, for example, a survival assay.

The terms “chemical compound”, “small molecule”, “chemical molecule” “small chemical molecule” and “small chemical compound” are used interchangeably herein and are understood to refer to chemical moieties of any particular type which may be synthetically produced or obtained from natural sources and usually have a molecular weight of less than 2000 daltons, less than 1000 daltons or even less than 600 daltons.

Hypoxia has been recognised as a key element in the pathomechanism of quite a number of diseases such as stroke, emphysema and infarct which are associated with sub-optimum oxygen availability and tissue damaging responses to the hypoxia conditions. In fast-growing tissues, including tumor, a sub-optimum oxygen availability is compensated by undesired neo- angiogenesis. Therefore, at least in case of cancer diseases, the growth of vasculature is undesired.

In view of this, the inhibition of angiogenesis and vascular growth, respectively, is subject to intense research. Already today some compounds are available which inhibit undesired angiogenesis and vascular growth. Some of the more prominent compounds are those inhibiting VEGF and the VEGF receptor. In both cases, the effect of VEGF is avoided by either blocking VEGF as such, for example by using an antibody directed against VEGF such as pursued by Genentech's AVASTIN (monoclonal AB specific for VEGF) (Ferrara N.; Endocr Rev. 2004 August;25(4):581-611), or by blocking the corresponding receptor, i.e. the VEGF receptor (Traxler P; Cancer Res. Jul. 15, 2004;64(14):4931-41; or Stadler WM et al., Clin Cancer Res. May 15, 2004;10(10):3365-70).

As, however, angiogenesis and the growth of vasculature is a very basic and vital process in any animal and human being, the effect of this kind of compound has to be focused at the particular site where angiogenesis and vascular growth is actually undesired which renders appropriate targeting or delivery a critical issue in connection with this kind of therapeutic approach.

It is thus an objective of the present invention to provide further means for the treatment of diseases involving undesired growth of vasculature and angiogenesis, respectively.

By “small interfering RNA” (siRNA) is meant an RNA molecule which decreases or silences (prevents) the expression of a gene/mRNA of its endogenous cellular counterpart. The term is understood to encompass “RNA interference” (RNAi). RNA interference (RNAi) refers to the process of sequence-specific post transcriptional gene silencing in mammals mediated by small interfering RNAs (siRNAs) (Fire et al, 1998, Nature 391, 806). The corresponding process in plants is commonly referred to as specific post transcriptional gene silencing or RNA silencing and is also referred to as quelling in fungi. The RNA interference response may feature an endonuclease complex containing an siRNA, commonly referred to as an RNA-induced silencing complex (RISC), which mediates cleavage of single-stranded RNA having sequence complementary to the antisense strand of the siRNA duplex. Cleavage of the target RNA may take place in the middle of the region complementary to the antisense strand of the siRNA duplex (Elbashir et al 2001, Genes Dev., 15, 188). For recent information on these terms and proposed mechanisms, see Bernstein E., Denli A M., Hannon G J: The rest is silence. RNA. 2001 November;7(11):1509-21; and Nishikura K.: A short primer on RNAi: RNA-directed RNA polymerase acts as a key catalyst. Cell. November 16, 2001;107(4):415-8.

siRNAs may be used in the screening processes of the present invention. The assignee of the present invention has found that siRNAs which inhibit the expression of the RTP801L polypeptide are useful in the treatment of various diseases and conditions. In the context of the present invention, siRNAs known to inhibit the expression of RTP801L may be used as to competitive agents in the screening of chemical compounds or biological molecules which inhibit RTP801L (thereby competing with said siRNAs for RTP801L inhibition) or in the screening of chemical compounds or other molecules which enhance the expression or activity of RTP801L (thereby reversing the RTP801L inhibition effected by said siRNA molecules). For further information on RTP801L siRNAs and methods of examining the inhibition effected by these siRNAs, see PCT Application No. PCT/IL 2007/000695, assigned to the assignee of the present invention, which is hereby incorporated by reference in its entirety.

During recent years, RNAi has emerged as one of the most efficient methods for inactivation of genes (Nature Reviews, 2002, v.3, p.737-47; Nature, 2002, v.418,p.244-51). As a method, it is based on the ability of dsRNA species to enter a specific protein complex, where it is then targeted to the complementary cellular RNA and specifically degrades it. In more detail, dsRNAs are digested into short (17-29 bp) inhibitory RNAs (siRNAs) by type III RNAses (DICER, Drosha, etc) (Nature, 2001, v.409, p.363-6; Nature, 2003, 425, p.415-9). These fragments and complementary mRNA are recognized by the specific RISC protein complex. The whole process is culminated by endonuclease cleavage of target mRNA (Nature Reviews, 2002, v.3, p.737-47; Curr Opin Mol Ther. 2003 June;5(3):217-24).

For disclosure on how to design and prepare siRNA to known genes see for example Chalk A M, Wahlestedt C, Sonnhammer E L. Improved and automated prediction of effective siRNA Biochem. Biophys. Res. Commun. Jun. 18, 2004;319(1):264-74; Sioud M, Leirdal M., Potential design rules and enzymatic synthesis of siRNAs, Methods Mol Biol.2004;252:457-69; Levenkova N, Gu Q, Rux J J.: Gene specific siRNA selector Bioinfornatics. Feb. 12, 2004;20(3):430-2. and Ui-Tei K, Naito Y, Takahashi F, Haraguchi T, Ohki-Hamazaki H, Juni A, Ueda R, Saigo K., Guidelines for the selection of highly effective siRNA sequences for mammalian and chick RNA interference Nucleic Acids Res. Feb. 9, 2004;32(3):936-48. See also Liu Y, Braasch D A, Nulf C J, Corey D R. Efficient and isoform-selective inhibition of cellular gene expression by peptide nucleic acids Biochemistry, Feb. 24, 2004;43(7):1921-7. See also PCT publications WO 2004/015107 (Atugen) and WO 02/44321 (Tuschl et al), and also Chiu Y L, Rana T M. siRNA function in RNAi: a chemical modification analysis, RNA 2003 September;9(9):1034-48 and U.S. Pat. Nos. 5,898,031 and 6,107,094 (Crooke) for production of modified/more stable siRNAs.

In a preferred embodiment, the molecules identified according to the screening systems of the present invention down-regulate RTP801L function. Down-regulation of RTP801L function preferably happens by reduction in the level of expression at the protein level and/or the mRNA level, whereby such reduced level of expression, preferably at the protein level, can be as little as 5% and be as high as 100%, with reference to an expression under conditions where the nucleic acid according to the present invention is not administered or is not functionally active. Such conditions are preferably the conditions of or as present in an expression system, preferably an expression system for RTP801L. Such expression system is preferably a translation system which can be an in vitro translation system, more preferably a cell, organ and/or organism. It is more preferred that the organism is a multicellular organism, more preferably a mammal, whereby such mammal is preferably selected from the group comprising man, monkey, mouse, rat, guinea pig, rabbit, cat, dog, sheep, cow, horse, cattle and pig. In connection with the down-regulation it is to be acknowledged that said down-regulation may be a function of time, i.e. the down-regulation effect is not necessarily observed immediately upon administration or functional activation of the nucleic acids according to the present invention, but may be deferred in time as well as in space, i.e. in various cells, tissues and/or organs. Such deferment may range from 5%-100%, preferably 10 to 50%. It will be acknowledged by the ones skilled in the art that a 5% reduction for a longer time period might be as effective as a 100% reduction over a shorter time period. It will also be acknowledged by the ones skilled in the art that such deferment strongly depends on the particular functional nucleic acid actually used, as well as on the target cell population and thus, ultimately, on the disease to be treated and/or prevented according to the technical teaching of the present application. It will also be acknowledged by the ones skilled in the art that the deferment can occur at any level as outlined above, i.e. a deferment in function, whereby such function is any function exhibited by RTP801L, a deferment in protein expression or a deferment at mRNA expression level.

When a nucleic acid to be employed in the processes of the present invention is manufactured or expressed, preferably expressed in vivo, such manufacture or expression preferably uses an expression vector, preferably a mammalian expression vector. Expression vectors are known in the art and preferably comprise plasmids, cosmids, viral expression systems. Preferred viral expression systems include, but are not limited to, adenovirus, retrovirus and lentivirus.

Methods are known in the art to introduce the vectors into cells or tissues. Such methods can be found generally described in Sambrook et al., Molecular cloning: A Laboratory Manual, Cold Springs Harbour Laboratory, New York (1983, 1992), or in Ausubel et al., Current Protocols in Molecular Biology, John Wiley and Sons, Baltimore, Md., 1998.

Suitable methods comprise, among others, transfection, lipofection, electroporation and infection with recombinant viral vectors. In connection with the present invention, an additional feature of the vector is in one embodiment an expression limiting feature such as a promoter and regulatory element, respectively, that are specific for the desired cell type thus allowing the expression of the nucleic acid sequence according to the present invention only once the background is provided which allows the desired expression.

In a further aspect the present invention is related to a pharmaceutical composition comprising a molecule identified according to the methods of the present invention and/or a vector according to the present invention and, optionally, a pharmaceutically acceptable carrier, diluent or adjuvants or other vehicle(s). Preferably, such carrier, diluents, adjuvants and vehicles are inert, and non-toxic. The pharmaceutical composition is in its various embodiments adapted for administration in various ways. Such administration comprises systemic and local administration as well as oral, subcutaneous, parenteral, intravenous, intraarterial, intramuscular, intraperitonial, intranasal, and intrategral.

It will be acknowledged by one skilled in the art that the amount of the pharmaceutical composition and the respective nucleic acid and vector, respectively, depends on the clinical condition of the individual patient, the site and method of administration, scheduling of administration, patient age, sex, bodyweight and other factors known to medical practitioners. The pharmaceutically effective amount for purposes of prevention and/or treatment is thus determined by such considerations as are known in the medical arts. Preferably, the amount is effective to achieve improvement including but limited to improve the diseased condition or to provide for a more rapid recovery, improvement or elimination of symptoms and other indicators as are selected as appropriate measures by those skilled in the medical arts.

In a preferred embodiment, the pharmaceutical composition according to the present invention may comprise other pharmaceutically active compounds. Preferably, such other pharmaceutically active compounds are selected from the group comprising compounds which allow for uptake intracellular cell delivery, compounds which allow for endosomal release, compounds which allow for, longer circulation time and compounds which allow for targeting of endothelial cells or pathogenic cells. Preferred compounds for endosomal release are chloroquine, and inhibitors of ATP dependent H⁺ pumps.

The pharmaceutical composition is preferably formulated so as to provide for a single dosage administration or a multi-dosage administration.

It will be acknowledged that the pharmaceutical composition according to the present invention can be used for any disease which involves undesired development or growth of vasculature including angiogenesis, as well as any of the diseases and conditions described herein. Preferably, these kind of diseases are tumor diseases. Among tumor diseases, the following tumors are most preferred: endometrial cancer, colorectal carcinomas, gliomas, endometrial cancers, adenocarcinomas, endometrial hyperplasias, Cowden's syndrome, hereditary non-polyposis colorectal carcinoma, Li-Fraumene's syndrome, breast-ovarian cancer, prostate cancer (Ali, I. U., Journal of the National Cancer Institute, Vol. 92, no. 11, Jun. 7, 2000, page 861-863), Bannayan-Zonana syndrome, LDD (Lhermitte-Duklos' syndrome) (Macleod, K., supra) hamartoma-macrocephaly diseases including Cow disease (CD) and Bannayan-Ruvalcaba-Rily syndrome (BRR), mucocutaneous lesions (e.g. trichilemmonmas), macrocephaly, mental retardation, gastrointestinal harmatomas, lipomas, thyroid adenomas, fibrocystic disease of the breast, cerebellar dysplastic gangliocytoma and breast and thyroid malignancies (Vazquez, F., Sellers, W. R., supra).

The pharmaceutical composition according to the present invention can also be used in a method for preventing and/or treating a disease as disclosed herein, whereby the method comprises the administration of a pharmaceutical composition or medicament comprising a molecule identified according to the methods or processes of present invention for any of the diseases described herein. Additional pharmacological considerations, formulations and delivery modes are disclosed in PCT Publication No.WO06/023544A2, assigned to assignee of the instant application.

The synthesis of any of the nucleic acids described herein is within the skills of the one of the art. Such synthesis is, among others, described in Beaucage S. L. and Iyer R. P., Tetrahedron 1992; 48: 2223-2311, Beaucage S. L. and Iyer R. P., Tetrahedron 1993; 49: 6123-6194 and Caruthers M. H. et. al., Methods Enzymol. 1987; 154: 287-313, the synthesis of thioates is, among others, described in Eckstein F., Annu. Rev. Biochem. 1985; 54: 367-402, the synthesis of RNA molecules is described in Sproat B., in Humana Press 2005 Edited by Herdewijn P.; Kap. 2: 17-31 and respective downstream processes are, among others, described in Pingoud A. et. al., in IRL Press 1989 Edited by Oliver R. W. A.; Kap. 7: 183-208 and Sproat B., in Humana Press 2005 Edited by Herdewijn P.; Kap. 2: 17-31 (supra).

All analogues of, or modifications to, a polynucleotide may be employed with the present invention, provided that said analogue or modification does not substantially affect the lunction of the polynucleotide. The nucleotides can be selected from naturally occurring or synthetic modified bases. Naturally occurring bases include adenine, guanine, cytosine, thymine and uracil. Modified bases of nucleotides include inosine, xanthine, hypoxanthine, 2-aminoadenine, 6-methyl, 2-propyl and other alkyl adenines, 5-halo uracil, 5-halo cytosine, 6-aza cytosine and 6-aza thymine, psuedo uracil, 4- thiuracil, 8-halo adenine, 8-aiminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine. In addition, analogues of polynucleotides can be prepared wherein the structure of the nucleotide is fundamentally altered and that are better suited as therapeutic or experimental reagents. An example of a nucleotide analogue is a peptide nucleic acid (PNA) wherein the deoxyribose (or ribose) phosphate backbone in DNA (or RNA is replaced with a polyamide backbone which is similar to that found in peptides. PNA analogues have been shown to be resistant to degradation by enzymes and to have extended lives in vivo and in vitro. Further, PNAs have been shown to bind stronger to a complementary DNA sequence than a DNA molecule. This observation is attributed to the lack of charge repulsion between the PNA strand and the DNA strand. Other modifications that can be made to oligonucleotides include polymer backbones, cyclic backbones, acyclic backbones, thiophosphate-D-ribose backbones, triester backbones, thioate backbones, 5′-2′ bridged backbone, artificial nucleic acids, morpholino nucleic acids, locked nucleic acid (LNA), glycol nucleic acid (GNA), threose nucleic acid (TNA), arabinoside, and mirror nucleoside (for example, beta-L-deoxynucleoside instead of beta-D-deoxynucleoside).

The polypeptides employed in the present invention may also be modified, optionally chemically modified, in order to improve their therapeutic activity. “Chemically modified”—when referring to the polypeptides, means a polypeptide where at least one of its amino acid residues is modified either by natural processes, such as processing or other post-translational modifications, or by chemical modification techniques which are well known in the art. Among the numerous known modifications typical, but not exclusive examples include: acetylation, acylation, amidation, ADP-ribosylation, glycosylation, GPI anchor formation, covalent attachment of a lipid or lipid derivative, methylation, myristlyation, pegylation, prenylation, phosphorylation, ubiqutination, or any similar process.

Additional possible polypeptide modifications (such as those resulting from nucleic acid sequence alteration) include the following:

“Conservative substitution”—refers to the substitution of an amino acid in one class by an amino acid of the same class, where a class is defined by common physicochemical amino acid side chain properties and high substitution frequencies in homologous polypeptides found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution.

“Non-conservative substitution”—refers to the substitution of an amino acid in one class with an amino acid from another class; for example, substitution of an Ala, a class II residue, with a class III residue such as Asp, Asn, Glu, or Gln.

“Deletion”—is a change in either nucleotide or amino acid sequence in which one or more nucleotides or amino acid residues, respectively, are absent.

“Insertion” or “addition”—is that change in a nucleotide or amino acid sequence which has resulted in the addition of one or more nucleotides or amino acid residues, respectively, as compared to the naturally occurring sequence.

“Substitution”—replacement of one or more nucleotides: or amino acids by different nucleotides or amino acids, respectively. As regards amino acid sequences the substitution may be conservative or non-conservative.

By “homolog/homology”, as utilized in the present invention, is meant at least about 70%, preferably at least about 75% homology, advantageously at least about 80% homology, more advantageously at least about 90% homology, even more advantageously at least about 95%, e.g., at least about 97%, about 98%, about 99% or even about 100% homology. The invention also comprehends that these polynucleotides and polypeptides can be used in the same fashion as the herein or aforementioned polynucleotides and polypeptides.

Alternatively or additionally, “homology”, with respect to sequences, can refer to the number of positions with identical nucleotides or amino acid residues, divided by the number of nucleotides or amino acid residues in the shorter of the two sequences, wherein alignment of the two sequences can be determined in accordance with the Wilbur and Lipman algorithm ((1983) Proc. Natl. Acad. Sci. USA 80:726); for instance, using a window size of 20 nucleotides, a word length of 4 nucleotides, and a gap penalty of 4, computer-assisted analysis and interpretation of the sequence data, including alignment, can be conveniently performed using commercially available programs (e.g., Intelligenetics™ Suite, Intelligenetics Inc., Calif.). When RNA sequences are said to be similar, or to have a degree of sequence identity or homology with DNA sequences, thymidine (T) in the DNA sequence is considered equal to uracil (U) in the RNA sequence. RNA sequences within the scope of the invention can be derived from DNA sequences or their complements, by substituting thymidine (T) in the DNA sequence with uracil (U).

Additionally or alternatively, amino acid sequence similarity or homology can be determined, for instance, using the BlastP program (Altschul et al., Nucl. Acids Res. 25:3389-3402) and available at NCBI. The following references provide algorithms for comparing the relative identity or homology of amino acid residues of two polypeptides, and additionally, or alternatively, with respect to the foregoing, the teachings in these references can be used for determining percent homology: Smith et al., (1981) Adv. Appl. Math. 2:482-489; Smith et al., (1983) Nucl. Acids Res. 11:2205-2220; Devereux et al., (1984) Nucl. Acids Res. 12:387-395; Feng et al., (1987) J. Molec. Evol. 25:351-360; Higgins et al., (1989) CABIOS 5:151-153; and Thompson et al., (1994) Nucl. Acids Res. 22:4673-4680.

“Having at least X % homolgy”—with respect to two amino acid or nucleotide sequences, refers to the percentage of residues that are identical in the two sequences when the sequences are optimally aligned. Thus, 90% amino acid sequence identity means that 90% of the amino acids in two or more optimally aligned polypeptide sequences are identical.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New-York (1989, 1992), and in Ausubel et al., Current Protocols in Molecular Biology, John Wiley and Sons, Baltimore, Md. (1988).

Standard organic synthesis protocols known in the art not specifically described herein are generally followed essentially as in Organic syntheses: Vol.1-79, editors vary, J. Wiley, New York, (1941-2003); Gewert et al., Organic synthesis workbook, Wiley-VCH, Weinheim (2000); Smith & March, Advanced Organic Chemistry, Wiley-Interscience; 5th edition (2001).

Standard medicinal chemistry methods known in the art not specifically described herein are generally followed essentially as in the series “Comprehensive Medicinal Chemistry”, by various authors and editors, published by Pergamon Press.

The features of the present invention disclosed in the specification, the claims and/or the drawings may both separately and in any combination thereof be material for realizing the invention in various forms thereof.

### Example 1

General Materials and Methods

If not indicated to the contrary, the following materials and methods were used in Examples 1-6:

Cell Culture

The first human cell line, namely HeLa cells (American Type Culture Collection) were cultured as follows: Hela cells (American Type Culture, Collection) were cultured as described in Czauderna F et al. (Czauderna, F., Fechtner, M., Aygun, H., Arnold, W., Klippel, A., Giese, K. & Kaufmann, J. (2003). Nucleic Acids Res, 31,670-82).

The second human cell line was a human keratinozyte cell line which was cultivated as follows: Human keratinocytes were cultured at 37° C. in Dulbecco's modified Eagle medium (DMEM) containing 10% FCS.

The mouse cell line was B16V (American Type Culture Collection) cultured at 37° C. in Dulbecco's modified Eagle medium (DMEM) containing 10% FCS. Culture conditions were as described in Methods Find Exp Clin Pharmacol. 1997 May; 19(4):231-9:

In each case, the cells were subject to the experiments as described herein at a density of about 50,000 cells per well and the double-stranded nucleic acid according to the present invention was added at 20 nM, whereby the double-stranded nucleic acid was complexed using 1 μg/ml of a proprietary lipid.

Induction of Hypoxia-Like Condition

The cells were treated with CoCl₂ for inducing a hypoxia-like condition as follows: siRNA transfections were carried out in 10-cm plates (30-50% confluency) as described by (Czauderna et al., 2003; Kretschmer et al., 2003). Briefly, siRNA were transfected by adding a preformed lOx concentrated complex of GB and lipid in serum-free medium to cells in complete medium. The total transfection volume was 10 ml. The final lipid concentration was 1.0 μg/ml; the final siRNA concentration was 20 nM unless otherwise stated. Induction of the hypoxic responses was carried out by adding CoCl₂ (100 μM) directly to the tissue culture medium 24 h before lysis.

Preparation of Cell Extracts and Immuno Blotting

The preparation of cell extracts and immuno blot analysis were carried out essentially as described by Klippel et al. (Klippel, A., Escobedo, M. A., Wachowicz, M. S., Apell, G., Brown, T. W., Giedlin, M. A., Kavanaugh, W. M. & Williams, L. T. (1998). Mol Cell Biol, 18, 5699-711; Klippel, A., Reinhard, C., Kavanaugh, W. M., Apell, G., Escobedo, M. A. & Williams, L. T. (1996). Mol Cell Biol, 16, 4117-27). Polyclonal antibodies against full length RTP801 were generated by immunising rabbits with recombinant RTP801 protein producing bacteria from pET19-b expression vector (Merck Biosciences GmbH, Schwalbach, Germany). The murine monoclonal anti-p110a and anti-p85 antibodies have been described by Klippel et al. (supra).

### Example 2

Experimental Models and Methods

In-vivo and in-vitro models which are useful in the identification of compounds which modulate RTP801L and animal models which can be used for validation of the activity of said identified compounds and their therapeutic potential are disclosed in PCT application. No. PCT/IL 2007/000695, PCT Publication No.WO06/023544A2 and PCT application No. PCT/US2007/01468, all assigned to the assignee of the instant application.

### Example 3

Experimental Methods Used to Identify Tight-Junction Proteins

Permeability Experiments

EOMA cells stably infected with Lentivirus encoding shRNA 14 (aka REDD14, which decreases levels of the RTP801 polypeptide) and Lentivirus controls (empty vector; Luciferase shRNA encoding vector and “Yeast” siRNA encoding vector) were used in the experiment.

Permeability was measured using the kit “In vitro Vascular Permeability Assay Kit” ECM640, Chemicon. Cells were grown in an collagen-coated inserts, seeding density-100.000/insert. Growth—4 days, in DMEM medium with 10% FCS.

H2O2 (1-2 mM) was added after 4 d of growth for 2 h. Then medium was replaced with fresh medium containing FITC-dextran. Incubation was continued for 10-40 min and aliquots were taken for fluorescence measurements (485-530 nM)

Western Blotting

Cells were grown in 6 well plates in similar conditions as above (±H2O2), and were lysed in RIPA buffer containing protease inhibitor cocktail and phosphatase inhibitors. Protein extracts were separated on 6% PAGE-SDS gel and transferred onto nitrocellulose membrane.

The membrane was probed using anti-ZO-1 sc-8146 (Santa Cruz) and anti-Cingulin 36-4401 (Zymed).

The results are presented in FIG. 9 and demonstrate that down-regulation of RTP801 (using shRNA) causes up-regulation of ZO-1 and cingulin in response to hypoxia.

### Example 4

Experimental Results

A) Co-Immuno Precipitation

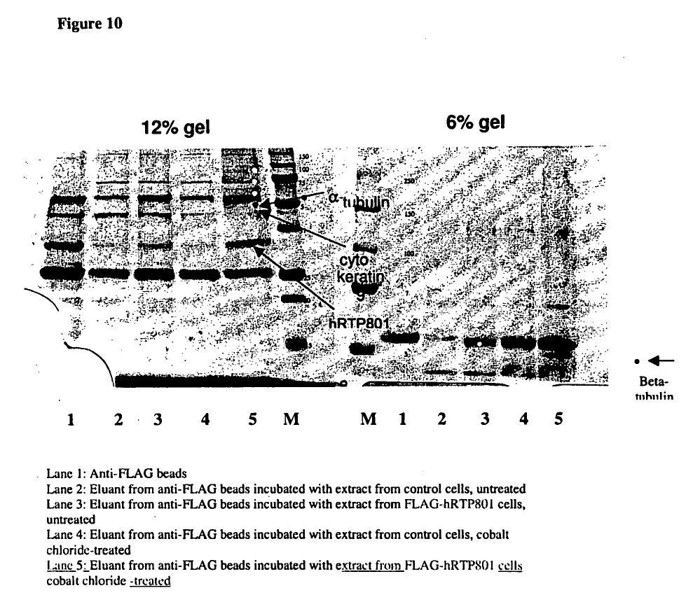

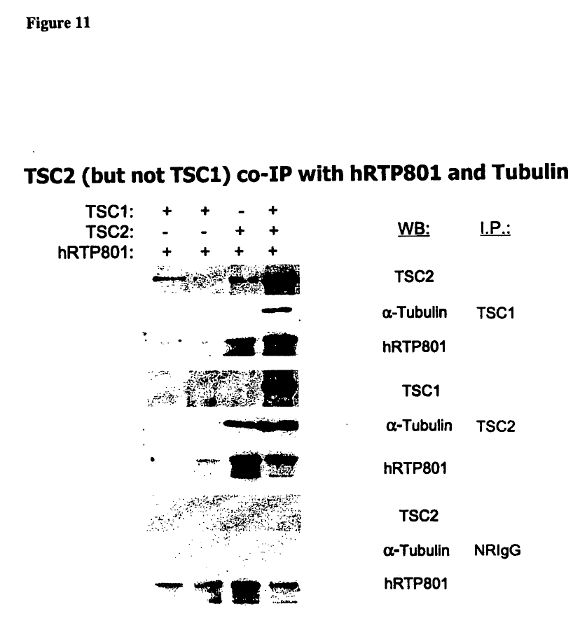

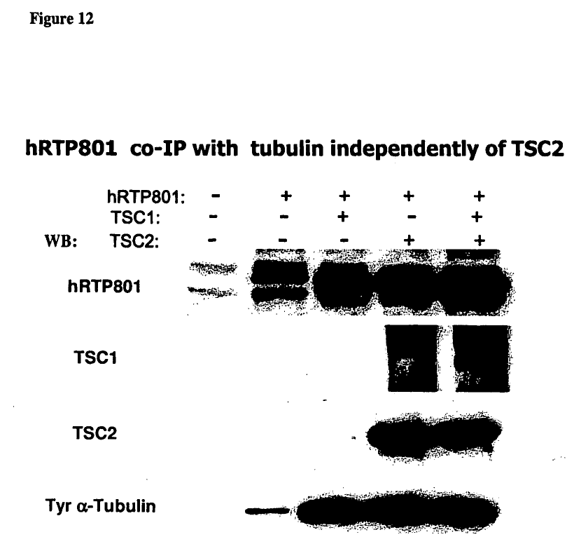

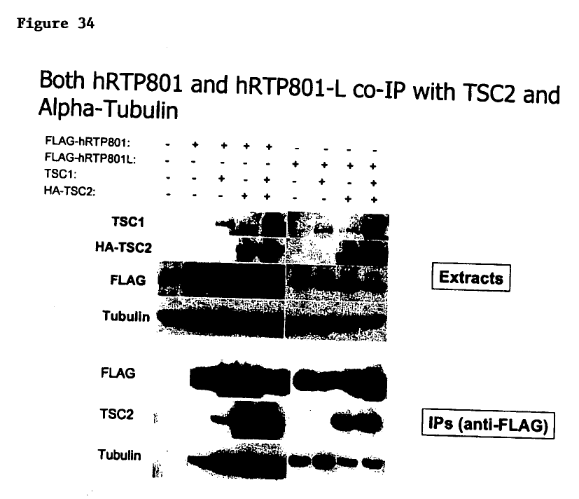

Description: 293T cells were transiently transfected with either empty plasmid or with plasmid containing FLAG-hRTP801 or FLAG-hRTP801-L (REDD2) cDNAs. 48 hrs. post-transfection, cobalt chloride (150 uM) was added (or omitted) for another 24 hrs. Cytosolic extracts prepared and IP was done using anti-FLAG antibodies. Alternatively, 293T cells were transiently transfected with plasmid containing FLAG-hRTP801 cDNA and plasmid containing TSC1 or TSC2 cDNAs or both. 48 hrs. post transfection, cobalt chloride (150 uM) was added for another 24 hrs. Cytosolic extracts prepared and IP was done using anti-TSC1, anti-TSC2 or normal rabbit IgG (NRIgG) antibodies. Alternatively, 293T cells were transiently transfected with either empty plasmid,plasmid containing FLAG-hRTP801 cDNA and plasmids containing either TSC1 and/or TSC2 cDNAs. 48 hrs. post transfection, cobalt chloride (150 uM) was added for another 24 hrs. Cytosolic extracts prepared and IP was done using anti-FLAG antibodies. Immunocomplexes were analysed by immunoblotting with the indicated antibodies (see FIGS. 10-12 and 34. The results are presented in FIGS. 10-12 and 34. FIG. 10 demonstrates that alpha/beta tubulin and cytokeratin-9 co-immunoprecipitate with RTP801. FIG. 11 demonstrates that TSSC2 co-immunoprecipitates with alpha tubulin and RTP801. FIG. 12 demonstrates that RTP801 co-immunoprecipitates with tubulin independently of exogenous TSC2. FIG. 34 demonstrates that RTP801 and RTP801-L co-immunoprecipitate with endogenous alpha tubulin and TSC2.

B) “Pull-Down” Experiments

Description: Recombinant hRTP801 (purified as a GST-fusion protein from bacteria) as well as free GST were used to capture interacting proteins from cell extract. GST or GST-hRTP801 were immobilized on glutathione beads and similar amount of each protein was incubated with various 293T cell extracts. Elution was done using reduced glutathione. Binding of TSC2 or alpha tubulin was assessed by Western Blotting with specific antibodies.

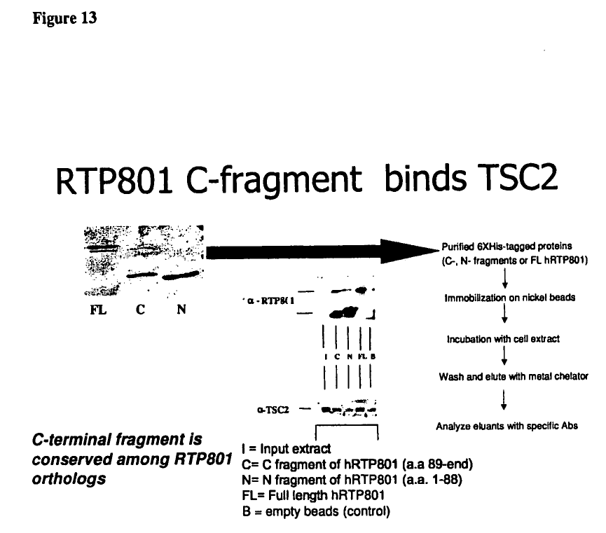

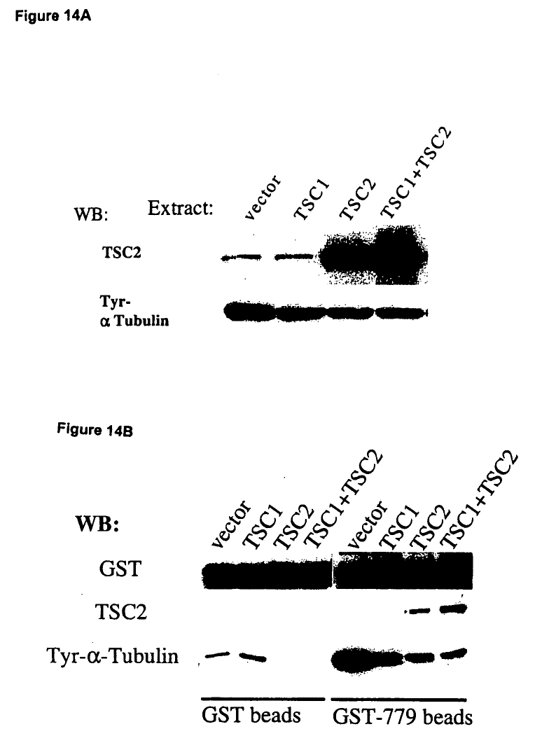

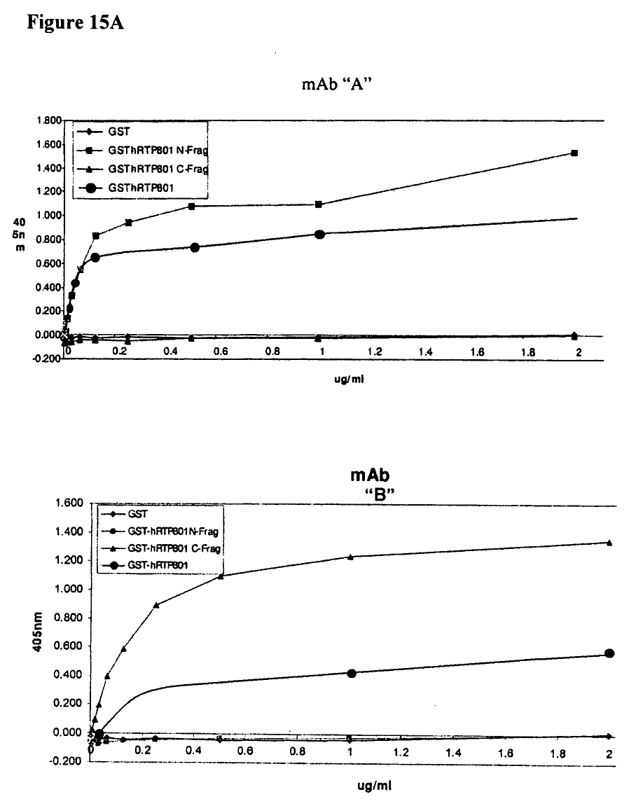

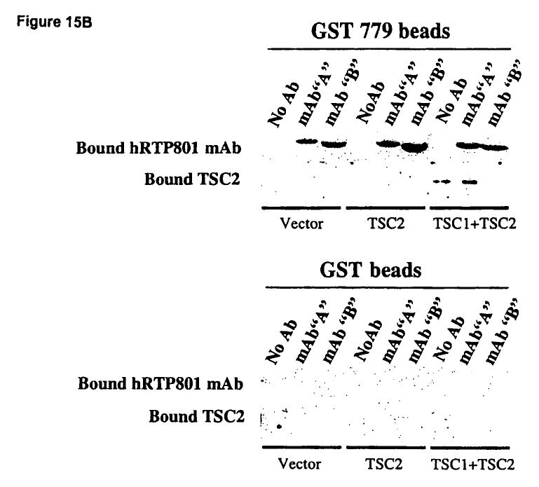

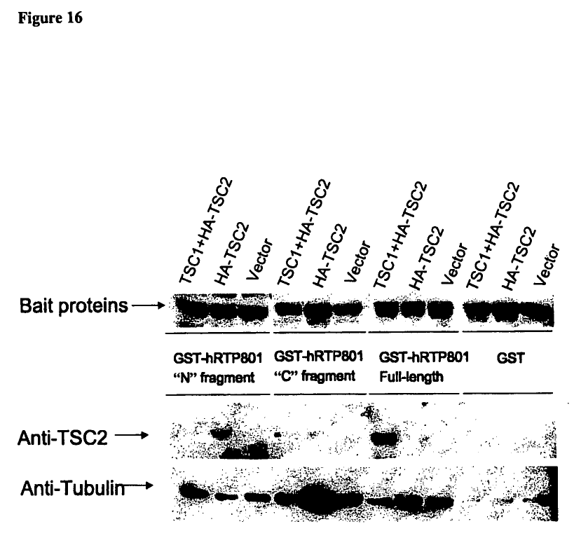

The results are presented in FIGS. 13-16. FIG. 13 demonstrates that RTP801 and RTP801 C-fragment but not RTP801 N-fragment bind TSC2 in vitro (“pull-down” from extract). FIG. 14 demonstrates that GST-RTP801 (but not free GST) binds to TSC2 and Tubulin in vitro. A. shows the Input extracts used for the experiment, while B. shows the pull-down results. FIG. 15 demonstrates that Monoclonal anti-hRTP801 C-fragment (termed mAb “B”) abolishes binding in vitro of GST-hRTP801 to TSC2 whereas monoclonal anti-hRTP801 N-fragment (termed mAb “A”) has no effect. A. Specificity of mAbs as judged by ELISA. B. Effect of pre-incubation with mAbs “A” or “B” on binding of GST-hRTP801 to TSC2. FIG. 16 demonstrates that binding of TSC2 to hRTP801 occurs within the C-fragment while binding of alpha tubulin to hRTP801 requires both C- and N-fragments.

C) Identification of TSC2 Fragment Sufficient for Interaction with RTP801

Description: 293T cells were transfected with plasmid containing FLAG-hRTP801 cDNA and one of the constructs shown in the figure. Cytosolic extracts were prepared and IP was done using anti-FLAG antibodies. Analysis of the immnocomplexes was done with anti-HA.

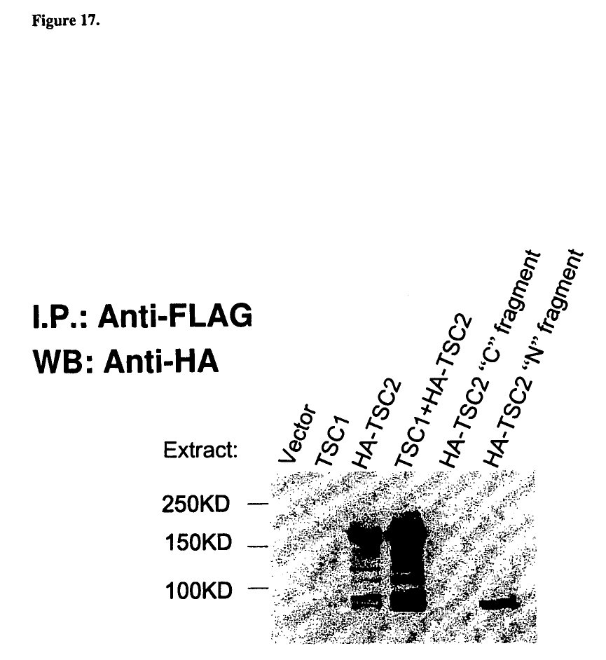

The results, showing that TSC2 “N” fragment (a.a. 2-935) is sufficient for interaction with FLAG-hRTP801, are presented in FIG. 17.

D) Up-Scaling of an Exemplary Screening Assay

Description: Purified hRTP801 (or as GST-hRTP801 ) is used to coat multi-well plates. Coating can either be directly or via anti-GST antibodies that are easily produced. Following a blocking step, small molecules are introduced followed by addition of extract from cells that express tagged TSC2 or TSC1/TSC2 complex. Following washes, bound TSC2 can be tested via its tag by an ELISA-based protocol. Wells which have a reduced signal contain inhibitory compounds which are thus identified.

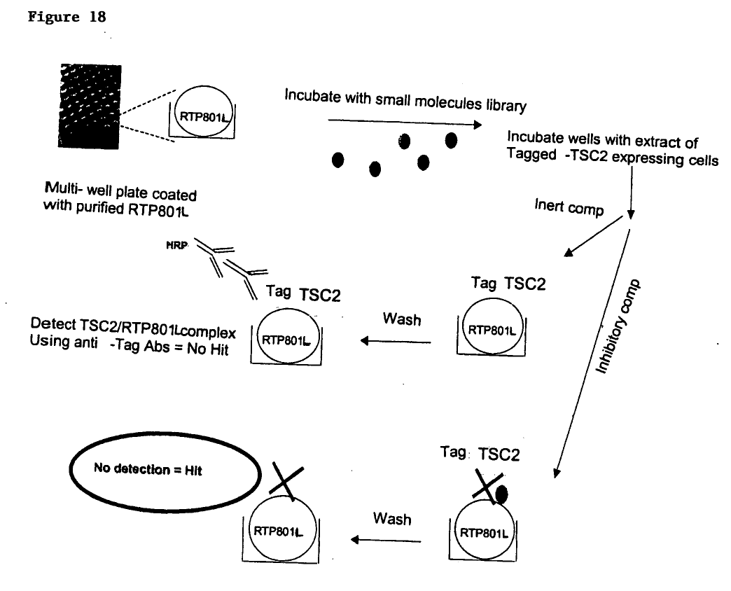

FIG. 18 is a schematic description of suggested ELISA-based assay for discovery of small molecules that can inhibit hRTP801/TSC2 complex.

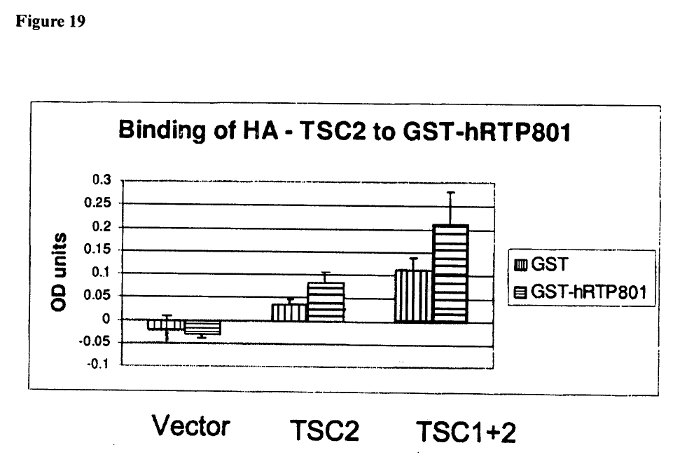

The validation results demonstrated in FIG. 19 show that Binding of HA-tagged TSC2 to GST-hRTP801 can be detected using an ELISA-based assay (as described above).

E) Binding of Purified Tubulin to RTP801

Description: Binding to purified tubulin (Cytoskeleton Inc.) was done essentially as decribed in Chen et al., JBC Vol. 281, pp. 7983-7993.

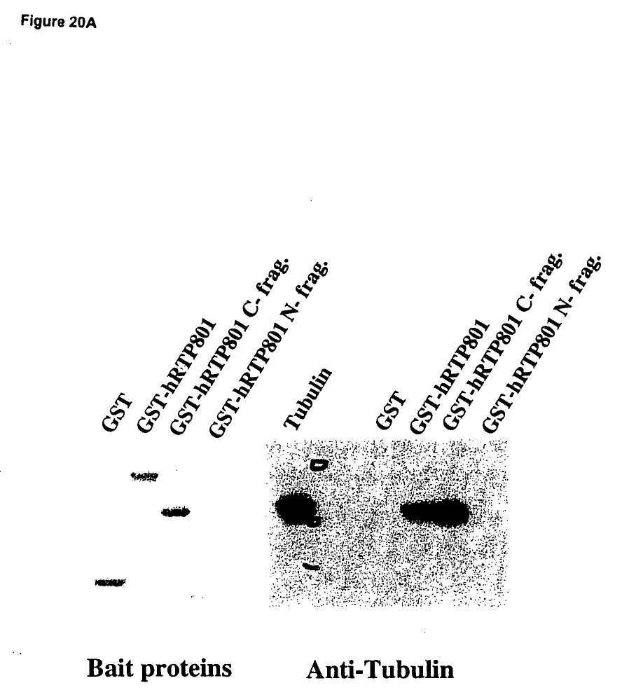

The results are presented in FIG. 20. Binding of purified tubulin to GST-hRTP801, GST-hRTP C-frag. and GST-hRTP801 N-frag. but not to free GST. A. Purified tubulin binds to both full hRTP801 and to its C-frag. A second experiment performed with a higher amount of the N-frag. Shows that the N-frag. also binds tubulin (B.).

### IN SUMMARY

Alpha/beta tubulin and cytokeratin 9 were discovered to be proteins that co-immuno precipitate with FLAG-hRTP801.

FLAG-hRTP801 and FLAG-hRTP801 -L co-immuno precipitate with endogenous alpha tubulin and TSC2

Exogenous TSC2 co-immuno precipitates with alpha tubulin and FLAG-hRTP801

hRTP801 co-immuno precipitates with tubulin independently of exogenous TSC2

TSC2 binds in vitro to 6× His-hRTP801 and 6× His-hRTP801 C-fragment (but not 6× His hRTP801 N-fragment)(“pull-down” from extract)

TSC2 and to tubulin bind in vitro to GST-hRTP801 (but not of free GST).

Monoclonal anti-hRTP801 C-fragment (termed mAb “A”) abolishes binding in vitro of GST-hRTP801 to TSC2 whereas monoclonal anti-hRTP801 N-fragment (termed mAb “B”) has no effect.

Binding of TSC2 to hRTP801 occurs at the C-fragment while binding of alpha tubulin to hRTP801 requires both C- and N-fragments.

TSC2 “N” fragment (a.a. 2-935) is sufficient for interaction with FLAG-hRTP801.

GST-hRTP801 (full length, C-fragment and N-fragment) Binds in vitro to purified brain tubulin

ELISA-format assay is effective for measuring thr binding of HA-TSC2 to GST-hRTP801.

The inventors of the present invention have thus shown that hRTP801L and hRTP801 both bind TSC2 and Tyr-tubulin. It has been demonstrated that RTP801 and RTP-801L both inhibit the mTOR pathway (Corradetti et al. The Stress-inducted Proteins RTP801 and RTP801L Are Negative Regulators of the Mammalian Target of Rapamycin Pathway J. Biol. Chem., Vol. 280, Issue 11, 9769-9772, Mar. 18, 2005). In addition, the inventors of the present invention have found, as disclosed herein, that a.a 161-195 of hRTP801 are sufficient for TSC2 binding and are essential for self-interaction. This region is very conserved between hRTP801 and hRTP801L. Therefore, without being bound by theory, hRTP801 and hRTP801L are functionally similar to each other, and inhibition of both hRTP801 and hRTP801L is more effective than inhibition of either one alone.

### Example 5

Further Experimental Results Relating to RTP801 Self-Association

A) hRTP801 Self Associates and the Region Between a.a 161-195 is Essential for Self-Association

293T HEK cells were co-transfected with a plasmid containing cDNA of HA-SV5-full length hRTP801 (“Prey”) as well as plasmid containing cDNA of one of the following: FLAG-full length hRTP801, FLAG-(C) hRTP801, FLAG-(N-C1) hRTP801, FLAG-(N-C1) hRTP801, FLAG-(N-C2)hRTP801 and FLAG-(C3) hRTP801. Forty-eight hours after transfection, cells were treated with 150 uM cobalt chloride for 18 hrs to mimick hypoxic stress conditions. The next day, cytosolic extracts were made by mechanic lysis under hypotonic conditions. FLAG-tagged bait proteins were immunoprecipitated with M2 anti-FLAG resin (Sigma). Following extensive washing, immunoprecipitated material was analyzed by immunblotting with either anti-hRTP801 polyclonal antibodies (proprietary) or with anti-SV5 polyclonal antibodies (AbCam).

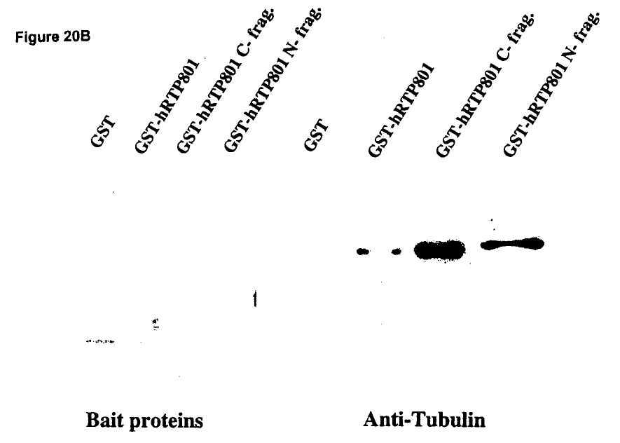

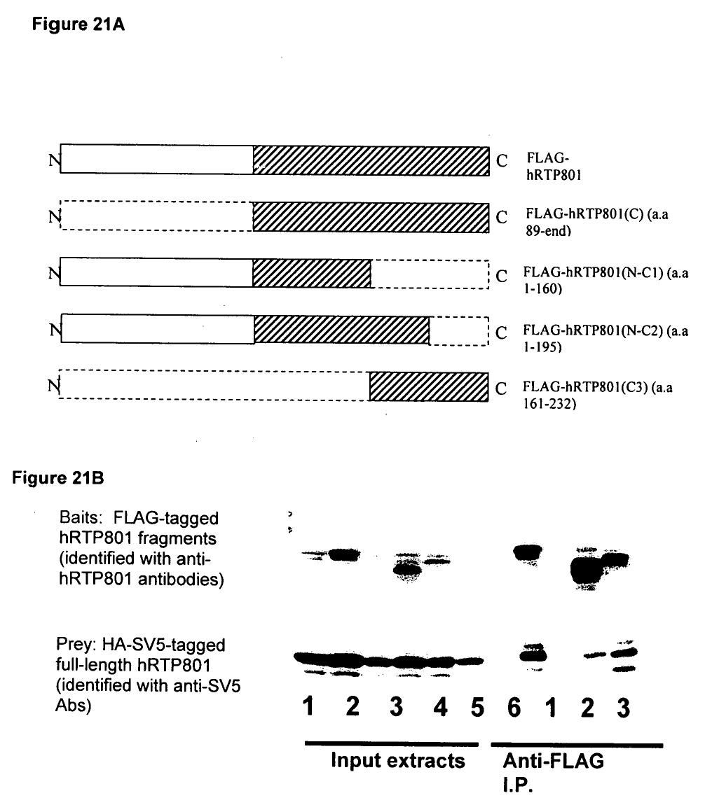

As shown in FIG. 21, HA-full length hRTP801 co-immuno-precipitated with FLAG-hRTP801, indicating self association of hRTP801 (lane 2, right panel). Moreover, hRTP801 N-C1 fragment lacking the last 72 a.a was markedly impaired in its ability to associate with the full-length hRTP801. hRTP801 N-C2 fragment lacking only the last 37 a.a was almost as efficient as the full-length protein in self association (lane 4, right panel). Thus, a.a 161-195 of hRTP801 are important for self association.

B) A Deletion Mutant of hRTP801 that is Defective in Self Association is Functionally Impaired

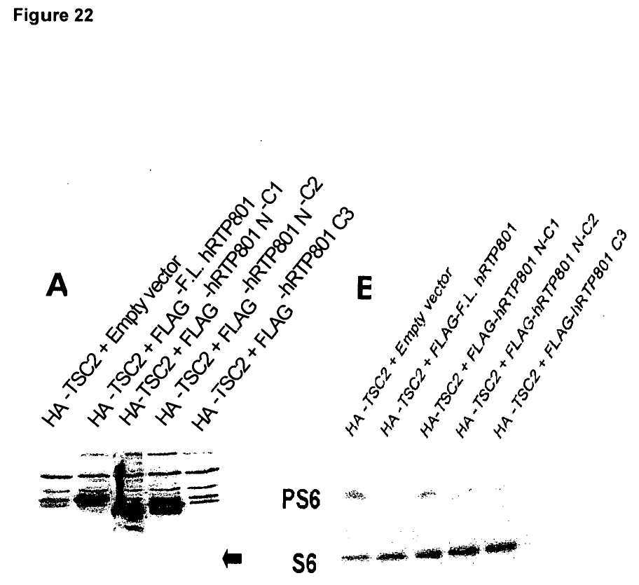

The Experiment was performed essentially as described in A) above, except cells were transfected with HA-TSC2 cDNA in addition to the hRTP801 constructs. Cell extracts were analyzed by anti-FLAG for expression of the FLAG-hRTP801 proteins (panel A) and by anti-phospho-S6 (pS6) which serves as a commonly used marker for mTOR activity (Averous J & Proud C G, Oncogene (2006) 25, 6423-6435). As a normalizer, total S6 antibody was used. As shown in FIG. 22 panel B, pS6 was absent in cells expressing full-length hRTP801 whereas cells expressing the hRTP801 N-C1 mutant (which is impaired in its ability to self associate), displayed similar amount of pS6 as control cells. In contrast, cells expressing hRTP801 N-C2 mutant (which was almost as efficient as full-length in self association) had lower level of pS6 than control. Interestingly, hRTP801 C3 fragment (a.a 161-232) was as efficient as hRTP801 N-C2 fragment (a.a 1-195) in inhibition of pS6 despite its very low expression (see in panel A). Thus, a.a 161-195 of hRTP801 are important for function of hRTP801 and its inhibition of mTOR activity.

Note that since RTP801 and RTP801L are homologous and share functional similarity, the fragments of RTP801L which are parallel to the RTP801 fragments tested above are also useful and novel and can be used in the screening systems of the present invention. Additionally, as will be demonstrated in d) below, RTP801L also associates with itself and with RTP801, and this can also be used as the basis for the screening systems of the present invention.

C) HTRF Measurement of hRTP801 Self Association

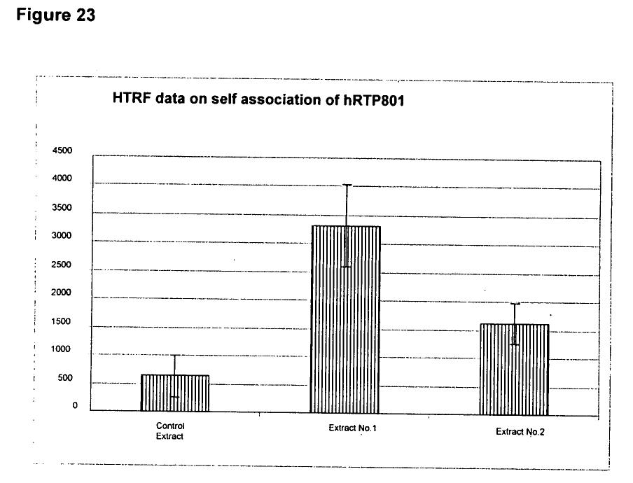

Self association of hRTP801 by was tested with HTRF technology (Jia Y, et al., “I-lomogeneous time-resolved fluorescence and its applications for kinase assays in drug discovery” Anal Biochem. Sep. 15, 2006;356(2):273-81. Epub 2006 May 24; Gabourdes et al., “A homogeneous time-resolved fluorescence detection of telomerase activity” Anal Biochem. Oct. 1, 2004;333(1):105-13). Eu-labeled anti-HA and XL665-labeled anti-FLAG antibodies (CisBio) were added at a 1:100 dilution to 6 ug cytosolic extract of 293T HEK cells that were transfected with either empty plasmid (control) or co-transfected with two plasmids each containing cDNAs of either FLAG-full length hRTP801 or HA-SV5-hRTP801. Following overnight incubation at 40C, the samples were excited at 330 nm and emission was read at 615 nm (Eu) and at 665 nm (FRET by XL665). The units shown in FIG. 23 refer to ratio of readings at 665 nm/615 nm*10⁴ factor. Two batches of extracts expressing hRTP801 with both tags were tested.

As shown in FIG. 23, FRET between Eu-anti HA and XL665-anti-FLAG were measured in extracts of cells that were transfected with the HA-hRTP801 and FLAG-hRTP801 cDNAs but not in control cells. Thus, self association of hRTP801 can be measured in an HTRF-based assay.

D) Bacterial Two-Hybrid Screening of RTP801 and RTP801L Association

The Bacterial 2-Hybrid System provides a rapid, cost effective and powerful tool for identifying and optimizing of different kinds of protein-protein interactions. The system is based on protein fragment complementation assay (PCA): two enzyme fragments are each fused to one interacting protein. An interaction between the two proteins leads to dimerization (assembly) of the 2 enzyme fragments and to the reconstruction of enzymatic activity. The system with which the results were obtained uses the Beta-Lactamase enzyme as a reporter with a detectable activity rendering Ampicillin resistance to host bacterial cells. The system is essentially composed of two plasmids, pALFA and pOMEGA, each one carrying a domain of the b-lactamase protein. Each domain is expressed and secreted into the periplasmic space of E. coli bacteria. If two interacting partners are fused with the b-lactamase fragments, the system will allow the positive selection of the interaction reconstituting the ampicillin resistance in bacterial cell.

The following interactions were tested in the baterial two-hybrid system:

RTP801 self interaction;

RTP801L (DDIT4L-Redd2) self interaction;

cross-interaction between RTP801 and RTP801L:

Map interacting domains (N/C×N/C for either protein)

The Following Control DNA Vectors Were Used:

1) pOMEGA-RTP801-SKP

2) pOMEGA RTP801-SKP_FKPA

3) pOMEGA Redd2_SKP

4) pOMEGA Redd2_SKP_FKPA

DNA was transformed into DH5AF′ and several colonies tested by DNA fingerprinting confirming insert size ands sequence.

Control of Fusion Protein Expression Level

Expression level of the fusion protein OMEGA-X (RTP801 or REDD2) was checked both at the total bacterial level and for periplasmic space expression. Experiments were repeated twice.

Total amount of bacteria was normalized and loaded on a SDS page. WB was developed with SV5 Tag.

Performing PCA Interaction

On the basis of WB expression level, the following vectors were selected for PCA experiments:

1—pOMEGA_RTP801_SKP

2—pOMEGA_Redd2_SKP_FKPA.

Bacteria containing the 3 different pAlfa vector s (RTP801; Redd2 and DELTAG (a negative control—a cholera toxin protein of 15 Kd) were co-transformed with the pOmega vectors and then plated on the selective medium.

As positive control known interactors (coiled coil domains) Alfa-E/Omega K were used. Bacteria were plated on different AMP concentrations (30/50100 μg/ml AMP) and different IPTG concentrations. (1 mM and 0,2 mM), and the experiment was repeated 3 times. SUMMARY OF THE RESULTS

All 9 combinations of interactors grow on double selection media (kanamicin and chloraphenicol), meaning all proteins were properly expressed.

All 4 combinations of pAlfa-RTP801/redd2 vs pOmega-RTP/redd2 interactions grew on 30 and 50 μg/ml of ampicillin, indicating reporter protein re-constitution meaning that the 2 tested proteins interact:

| | pOMEGA_RTP801 | pOMEGA-Redd2 | pOMEGA-2.8 |
|---|---|---|---|
| pALFA-RTP | + | + | − |
| pALFA-Redd2 | + | + | − |
| pALFA-deltaG | − | +/− | − |

All the controls for interaction were negative, excluding a low background for the combination pOmega-redd2/pAlfa-DG (which disappeared at 50 μg/ml).

### Example 6

Additional Results and Assays

Without being bound by theory, the inventors of the present invention have discovered the following:

1. RTP801 forms a physical complex with TSC2; interaction between RTP801 and TSC2 occurs via the C-terminal domain of RTP801 and N-terminal half of TSC2. For the purpose of a screening assay, recombinant bacterially expressed RTP801 can bind TSC2 expressed in cells.

2. RTP801 forms a physical complex with tyrosinated alpha-tubulin (Tyr-tubulin), and both N- and C-terminal fragments of RTP801 can bind Tyr-tubulin. Recombinant RTP801 or its C-terminal fragment can directly interact with purified tubulin.

3. Further, it was noted that RTP801-TSC2 and RTP801-tubulin complexes are separate entities and, moreover, mutually exclusive.

4. RTP801 and RTP801L can associate with each other and self associate.

The Following is a Non Exclusive List of Possible Screening Assays which can be Conducted Utilizing RTP801L:

a. ELISA-based assay utilizing immobilized GST-RTP801L baits and protein extracts from HA-TSC2 overexpressing cells—disruption of RTP801L-TSC2 interaction.

b. ELISA-based assay utilizing immobilized purified tubulin as a bait and recombinant GST-RTP801L—disruption of RTP801-tubulin interaction.

c. FRETWorks S.Tag based assay utilizing immobilized GST-RTP801L baits and extracts of cells overexpressing S-tagged TSC2—disruption of RTP801L-TSC2 interaction.

d. FRETWorks S.Tag based assay utilizing immobilized tubulin as a bait and recombinant S-tagged RTP801L (or fragments thereof)—disruption of RTP801L—tubulin interaction.

The above assays can also be used as secondary assays to test the function of small molecules identified, potentially, in a “Neogenesis-type” assay (identification of small molecules that directly bind to recombinant RTP801L protein).

Additional Assays which may be Employed Include:

1. Cell free assay utilizing recombinant minimal interacting fragments of RTP801L and TSC2—as described herein—disruption of RTP801L-TSC2 interaction.

2. Cell-free assay utilizing differently tagged recombinant RTP801L proteins or fragments thereof—disruption of RTP801L self-association.

RTP801L-TSC2 Interaction

Background

Without being bound by theory, RTP801L is involved in the mammalian target of rapamycin (mTOR) pathway. Specifically, RTP801L, whose expression is induced under a variety of cell stresses, is importantfor inhibition of activity of mTOR rapamycin-sensitive complex 1 under stress conditions such as hypoxia or energy deprivation. The exact molecular mechanism via which RTP801L inhibits mTOR activity remains obscure. However, it has been shown that RTP801L acts upstream to mTOR and exerts its inhibitory activity in a strict dependence on tuberin (TSC2) (Sofer et al ). TSC2 serves as a GTPase activating protein (GAP) for Rheb, a membrane-bound GTPase which, when in an active GTP-bound state, can activate the mTOR kinase (Zhang et al, Tee et al). As a consequence, activation of TSC2 leads to mTOR inhibition. TSC2 regulates Rheb function in cell membranes where Rheb resides. Lacking its own membrane targeting motifs, TSC2 is held in the membranes via interaction with hamartin (TSC1). Phosphorylation of TSC2 by AKT leads to its dissociation from TSC1, translocation to the cytosol and subsequent degradation (Cai et al).

Since RTP801L and TSC2 are functionally linked and both act to inhibit mTOR activity, it is possible to inhibit RTP801L by decoupling it from TSC2.

Results Relating to the RTP801 and TSC2 Interaction

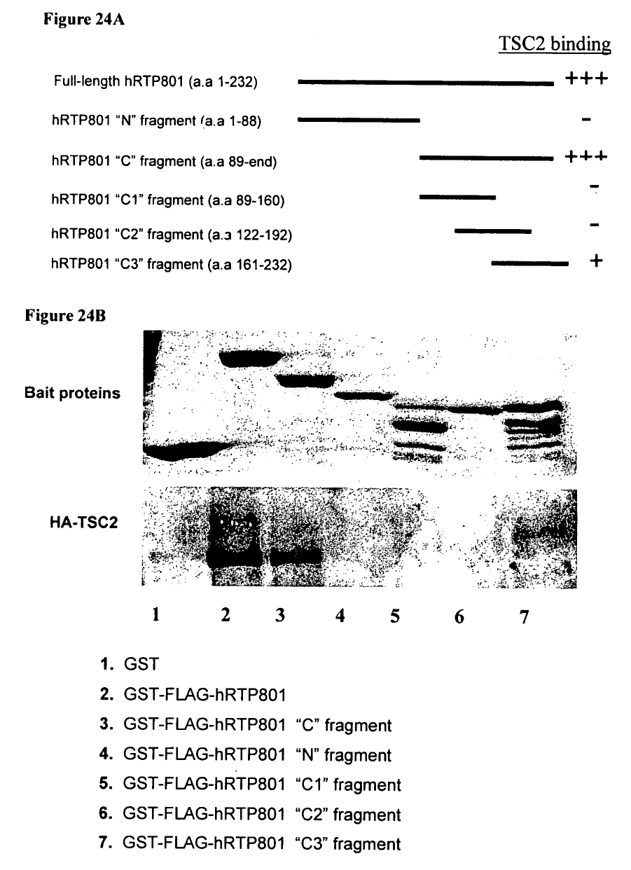

hRTP801 region that binds TSC2 (FIG. 24). Various cDNA fragments of hRTP801 (FIG. 24A) were subcloned into a pGEX6P plasmid, to produce GST-FLAG fusion proteins which were purified on glutathione resin (FIG. 24B). The purified GST-FLAG fusion proteins (“baits”) were immobilized to glutathione resin and incubated with post-nuclear supernatant of 293T cells transfected with either HA-tagged TSC2 (HA-TSC2) or with empty plasmid. Following elution, column-bound HA-TSC2 was then detected by immunoblotting with anti-HA antibodies. As shown in FIG. 24, both GST-full length hRTP801 and GST-hRTP801 “C” fragment bound HA-TSC2 present in the cell extract (lanes 2 and 3, respectively), while free GST (lane 1) failed to do so. Notably, GST-hRTP801 “C3” bait encompassing the last 70 a.a of hRTP801 was able to bind HA-TSC2 ailbeit with lower efficiency (lane 7). In contrast, GST-hRTP801 “N” and GST-hRTP801 “C1” and GST-hRTP801 “C2” baits, failed to bind HA-TSC2 (lanes 4, 5, 6, respectively). Thus, the last 70 amino acids of hRTP801 comprising the C3 fragment are sufficient to bind TSC2. Note that the C-terminal domain of RTP801 is the most conserved portion among all RTP801 orthologues. Similar results are achieved with RTP801L.

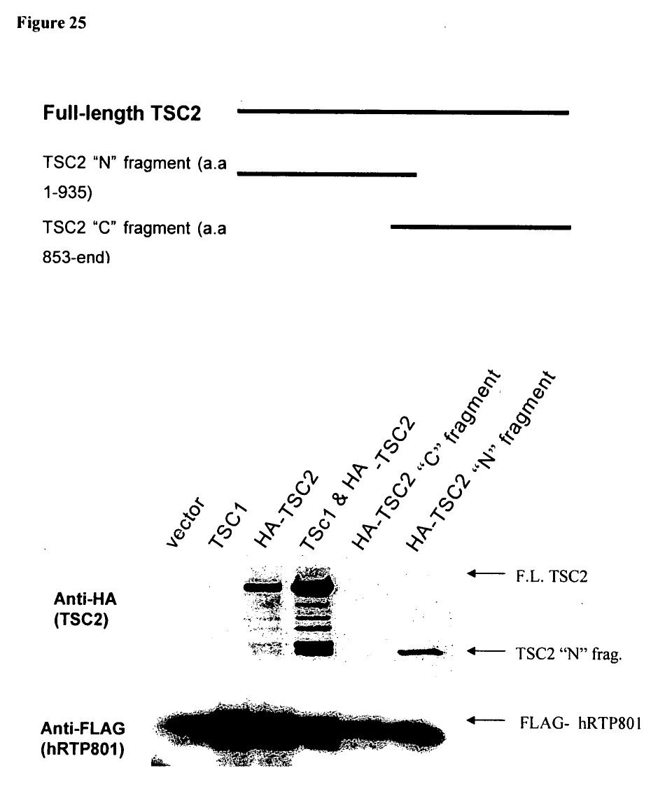

TSC2 region that binds hRTP801 (FIG. 25). Human TSC2 HA-tagged “N” and “C” fragments (FIG. 25, upper panel) as well as full length HA-tagged TSC2 were transfected into 293T cells along with FLAG-hRTP801 or with empty vector. Forty-eight hours after transfection, the cells were treated with CoCl₂ for overnight. Cells were harvested and post-nuclear supernatant was prepared and used for IP with anti-FLAG antibodies. As shown in FIG. 4, FLAG-hRTP801 was co-IP with both full length HA-TSC2 and HA-“N” fragment of TSC2 (lower panel). Unfortunately, the HA-“C” fragment of TSC2 was poorly expressed (undetectable in input extracts following immunoblotting with anti-HA antibodies) and hence could not be tested for co-IP with hRTP801. Nevertheless, these results show that aa 1-935 of human TSC2 are sufficient to bind to hRTP801. Similar results are achieved with RTP801L

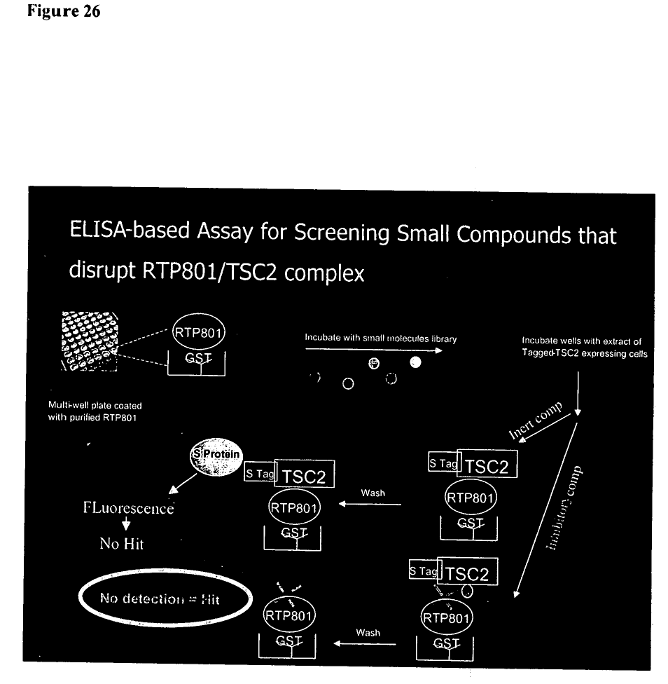

FIGS. 18 and 19 show schematic details of some of the bioassays proposed herein; anadditional possible proposed assay is shown in FIG. 26. This exemplary assay is based on the FRETWorks S-Tag assay kit sold by Novagen. Briefly, a protein of interest (in our case TSC2) is fused to a 15 aa-long peptide (S Tag). This peptide binds with nM affinity to a 104aa enzymatically inactive fragment of Rnase S (S protein). Upon binding, it reconstitutes a functional RNase S enzyme. The reconstituted enzymatic activity can then be assayed using a ribo-oligo substrate having a fluorophore group on one of its ends and a quencher group—on the other. Upon cleavage by the reconstituted RNase S, a fluorescence signal is obtained. Thus, as a modification of the first generation assay, S Tagged-TSC2-containing extract is allowed to bind GST-FLAG-hRTP801L bait bound to the plate. Bound S-tagged-TSC2 is assayed by a simple addition of the S protein and oligo-substrate followed by fluorescence measurement. This saves the need for the last 2 steps included in the first assay. Sensitivity of the assay may also be increased.

Note that screening assays employing any of the interactions disclosed herein can be performed along the lines of those exemplified in FIGS. 18, 19 and 26.

RTP801L-Tyr-Alpha-Tubulin Interaction

Background

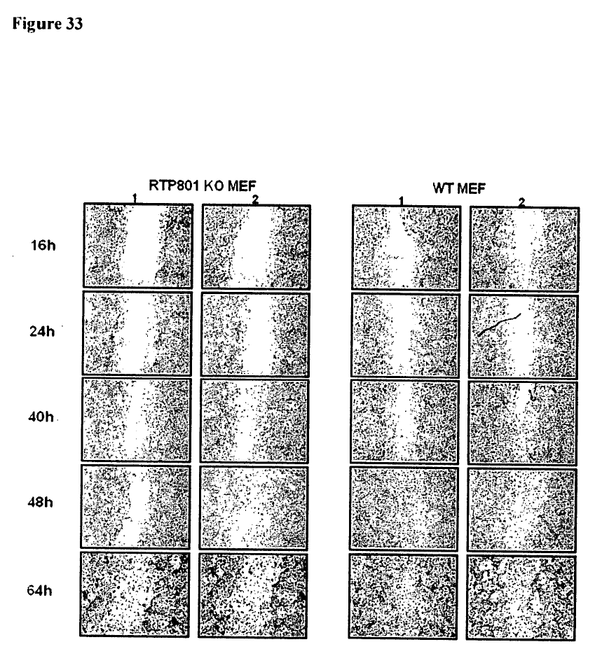

Alpha-Tubulin was identified by the inventors of the present invention as a protein that co-immunoprecipitated with FLAG-RTP801L from overexpressing cells. No functional linkage between RTP801L and cytoskeleton has been previously suggested in the literature. However, several lines of evidence suggest a functional connection between mTOR and TSC1/TSC2 complex with this subcellular compartment, involving both actin cytoskeleton and microtubules. Inhibition of mTOR complex 1 by rapamycin significantly affects microtubules assembly, elongation and stability (Choi et al). TSC1- and TSC2-null cells have disorganized microtubules and are defective microtubule-dependent protein transport (Jiang and Yeung). TSC1-and TSC2-null cells have altered distribution of actin filaments, which is reversed by either rapamycin or by Rheb inhibitors (Gau et al.). mTOR-rictor-bound complex, which is rapamycin-resistant, regulate the actin cytoskeleton (Sarbassov et al). There is also a compelling evidence that TSC1/TSC2 complex has an independent from mTOR activity impact on cytoskeleton through regulation of Rac1 and Rho small GTPases. Thus, inactivation of TSC2 complex leads to reduced Rho-GTPase activity, decreased actin stress fibers and focal adhesions, and reduced motility and invasion (Liu et al). Interestingly, our proprietary data demonstrates also a reduced motility of RTP801 KO mouse embryo fibroblasts (MEF) (FIG. 33) in a standard cell monolayer scratching assay. Similar results are achieved in RTP801L knock-out mice. This is in line with the fact that RTP801L acts as an activator of TSC1 /TSC2 complex under stress conditions. Reduced motility of cells with inhibited RTP801L may be relevant to quite a number of therapeutic outcomes associated with RTP801L inhibition: e.g., reduced tumor growth and metastasis, reduced infiltration of inflammatory cells in the tissues, reduced pathological neoangiogenesis.

RTP801 interacts specifically with tyrosinated alpha-tubulin (see below), and similar results are observed with RTP801L. Tubulin undergoes tyrosination at its carboxyl terminus. This tyrosination is reversible leading to two distinct populations of microtubules: one, composed of tyrosinated tubulin (Tyr-tubulin), is dynamic and prone to depolymerization and another one, composed of detyrosinated or Glu-tubulin, is more stable (Bulinski et al). There are several proteins known to bind preferentially to Tyr-tubulin (Peris et al). Interestingly, one of these proteins, CLIP-170, was also shown to bind mTOR (Choi et al.). Moreover, the inventors of the present invention have discovered that CLIP-170 associated protein (CLASP2) is elevated ˜3 folds in retinas of diabetic WT mice as compared with diabetic RTP801 KO mice whereas in non-diabetic mice its expression is unchanged in RTP801 KO mice compared to WT animals, and similar results are observed in RTP801L KO mice. Potential direct influence of RTP801L on microtubule dynamics may be of therapeutic importance influencing cell proliferation, motility and endothelial layers permeability (Birukova et al).

Results Relating to the RTP801 L and Alpha-Tubulin Interaction

A. Evidence of an hRTP801L—Tyr-Tubulin Complex

i. Co-IP of endogenous Tyr-tubulin with exogenous FLAG-tagged hRTP801L As shown in FIG. 34, tubulin was specifically co-immunoprecipitated with FLAG-hRTP801L.

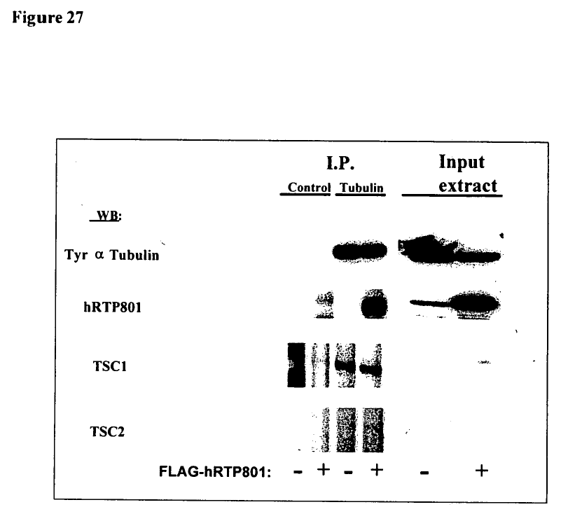

ii. Reciprocal co-IP of exogenous FLAG-hRTP801 with endogenous Tyr-tubulin (FIG. 12 and 27). Co-IP of endogenous Tyr-tubulin with exogenous FLAG-tagged hRTP801 (FIG. 12). As shown in FIG. 12, tubulin was specifically co-immunoprecipitated with FLAG-hRTP801. A reciprocal experiment was done essentially as described for FIG. 12 except that IP was performed using anti-Tyr-tubulin antibodies. As evident, hRTP801 was specifically and efficiently co-immunoprecipitated along with Tyr-tubulin where as no co-immunoprecipitation of RTP801 was observed with control antibodies.

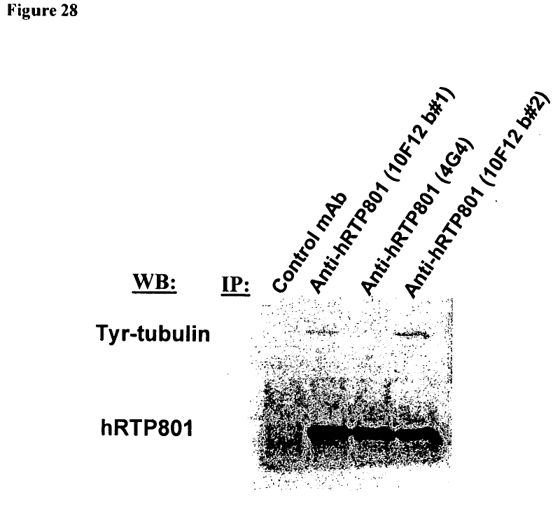

iii. Co-IP of endogenous Tyr-tubulin with endogenous hRTP801 (FIG. 28). Undifferentiated neuroblastoma cells (BE2C) were treated for 20 hrs with 150 uM CoCl₂ to stress the cells and to induce the expression of endogenous hRTP801. Post-nuclear supernatant was prepared and used for IP with either monoclonal antibodies (mAbs) against hRTP801 (two batches of mAb 10F12 and mAb 4G4) or with control monoclonal antibody. As evident, endogenous hRTP801 was specifically IP by both 10F12 and 4G4. Tyr-tubulin was co-IP with hRTP801 only when 10F12 mAb was used potentially indicating that mAb 4G4 interferes with RTP801-tubulin interactions. No co-IP of Tyr-tubulin was observed with control mAb.

B. Defining the Minimal Tubulin-Binding Regions in hRTP801

Pull-down of Tyr-tubulin from cell extract (FIG. 16). Various regions (N-erminus, C-terminus, full-length—for construct details, see FIGS. 13 and 24) of hRTP801 were cloned in pGEX6P plasmids and expressed as GST-FLAG fusion proteins in bacteria followed by purification on glutathione resin (upper panel). The purified GST-FLAG fusion proteins (“baits”) were immobilized on glutathione resin and incubated with post-nuclear supernatants of various transfectants of 293T cells (transfection details are irrelevant to this particular description). Following elution, RTP801-bound Tyr-tubulin was then detected by immunoblotting with anti-Tyr-tubulin antibodies. As evident, all RTP801 baits used were capable of Tyr-tubulin binding. Thus, Tyr-tubulin may bind hRTP801 in at least different two locations. Similar results are achieved with RTP801L.

C. Evidence of Direct Binding Between hRTP801 and Tyr-Tubulin

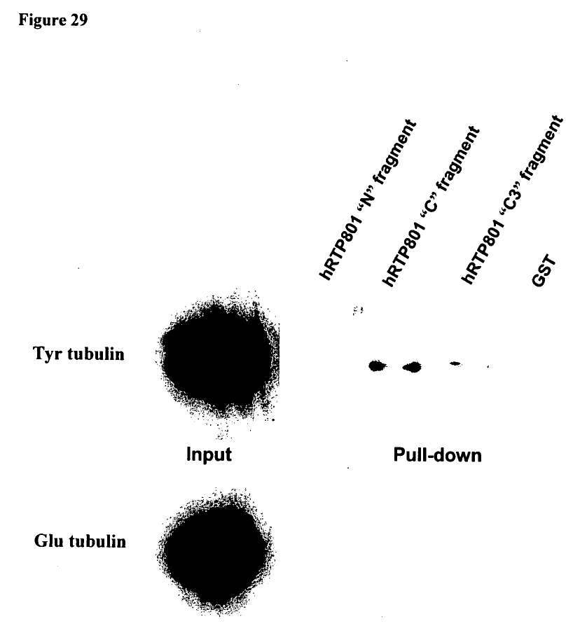

Direct binding of hRTP801 to Tyr-tubulin was assessed using ultra-pure brain tubulin (Cytoskeleton Inc., cat# TL238). Pull-down experiments using various GST-fused RTP801 baits were done essentially as described above except the fact that the beads with immobilized GST-RTP801 baits were incubated with purified tubulin under stringent conditions. Binding of Tyr-tubulin was assessed using specific anti-Tyr-tubulin antibodies. As shown herein, purified Tyr-tubulin bound to GST-FLAG-hRTP801 as well as to the hRTP801 “C” and “N” fragments but not to free GST. Thus, hRTP801 binds Tyr-tubulin directly. Results (FIG. 29) suggest that hRTP801 has preference for Tyr-tubulin as compared with detyrosinated tubulin (Glu-tubulin). This was determined by probing the hRTP801-bound purified tubulin with either Tyr-tubulin or Glu-tubulin antibodies. Similar results are achieved with RTP801L.

Development of an in vitro Bioassay for hRTP801L-Tyr-Tubulin Interaction

Of the many possible screening assays discussed herein, the two following assays were tested:
a. GST-hRTP801 was immobilized on an ELISA plate, incubated with purified tubulin and, following washes, bound Tyr-tubulin was detected using anti-Tyr-tubulin antibodies.

b. Purified tubulin was immobilized on an ELISA plate, incubated with purified GST-hRTP801 baits or with free GST. Following washes, bound GST-hRTP801 was detected using anti-GST antibodies.

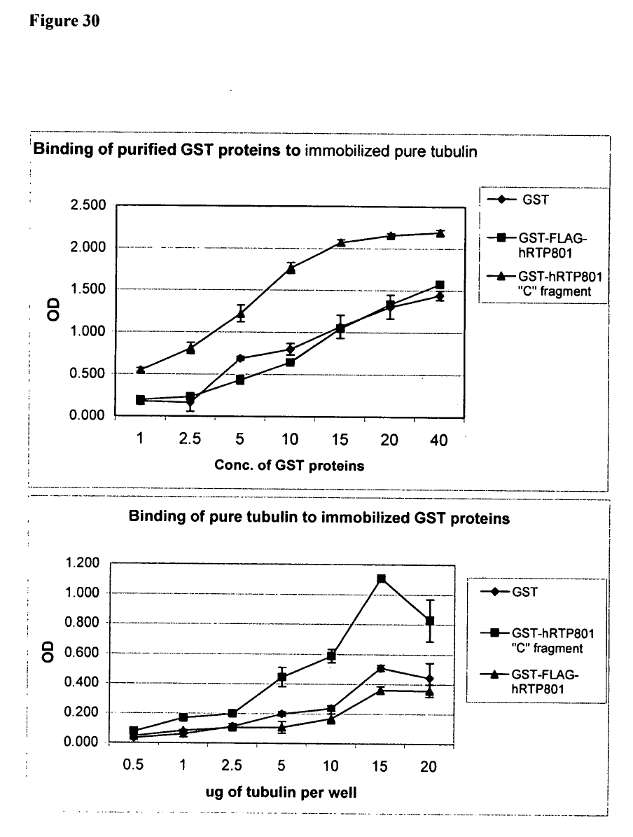

Preliminary results (FIG. 30). In the 96-well format experiment, the GST-FLAG-hRTP801 bait did not bind tubulin above control levels (free GST). In contrast, GST-hRTP801 “C” fragment displayed saturating binding curves in both assay types. Similar results are achieved with RTP801L.

An alternative assay may involve usage FRETWorks S Tag assay kit according to the principles described for RTP801L-TSC2 interaction above. However, in the case of
tubulin-RTP801L interaction, the plates will be coated with purified tubulin and
binding of S-tagged RTP801L will be assessed by monitoring RNase S activity.

TSC2-Tyr-Alpha Tubulin Interactions

Background

As discussed above, TSC2 null cells are defective in their cytoskeleton organization and microtubule-dependent transport (Jiang and Yeung). The inventors of the present invention were the first to discover physical association between the TSC1/TSC2 complex and tubulin.

Results

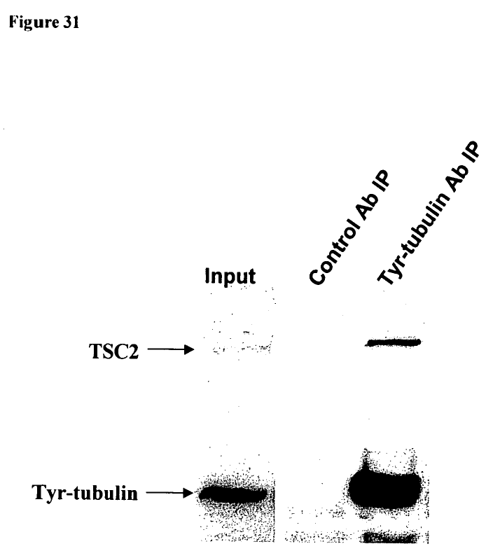

Endogenous TSC2 co-immunoprecipitated with endogenous Tyr-alpha-tubulin (FIG. 31). Briefly, 293T cells were treated with CoCl₂ as described above; post nuclear supernatant was prepared and used for IP with either control antibodies or anti-Tyr-tubulin antibodies. Co-immunoprecipitated proteins were identified using either anti-Tyr tubulin or anti-TSC2 antibodies. As evident, TSC2 was specifically co-immunoprecipitated with Tyr-alpha tubulin. Thus, the inventors of the present invention have demonstrated association of TSC2 with tubulin.

Interplay Between hRTP801, TSC2 and Tyr-Tubulin Complexes

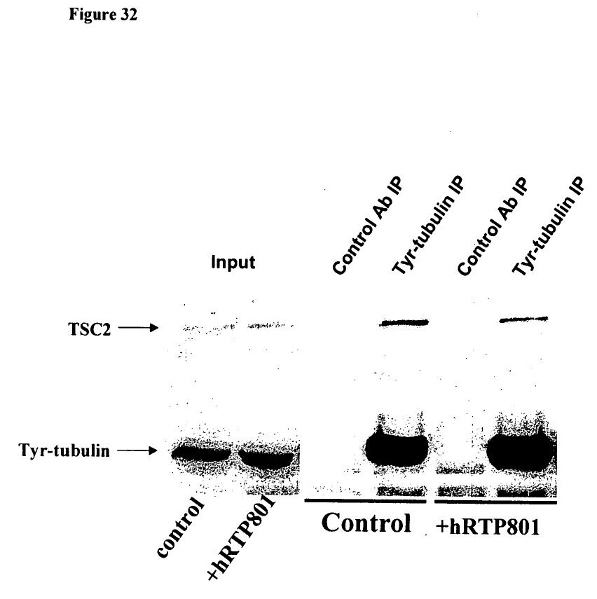

As Tyr-tubulin, TSC2 and RTP801 may interact with each other in a pair-wise manner, the inventors of the present invention examined whether hRTP801, TSC2 and Tyr-tubulin can affect the binding of each pair to the third binding partner. As shown in FIG. 32, co-IP of endogenous TSC2 with tubulin (conditions of experiment are as described for FIG. 31 except that exogenous hRTP801 was over-expressed for 48 hrs. prior to IP in a portion of the cells) was significantly reduced in the presence of overexpressed exogenous hRTP801.

As both tubulin and hRTP801 were probably in high excess over TSC2, it is likely that hRTP801 and tubulin competed for the binding on TSC2. Likewise, FIG. 16 shows reduced tubulin binding to GST-hRTP801 when TSC2 is bound (in HA-TSC2 overexpressing cells). Therefore, without being bound by theory there are separate mutually exclusive complexes of hRTP801-Tyr-tubulin, hRTP801-TSC2 and TSC2-Tyr-tubulin. Similar results are achieved with RTP801L.

RTP801L Self Association

Data obtained by the inventors of he present invention from bacterial two-hybrid system, suggests that hRTP801L forms homodimers (see Example 5). A screening assay may also be based upon inhibition of RTP801L function by abolishing homodimerization.

### REFERENCES

Brugarolas J, Lei K, Hurley R L, Manning B D, Reiling J H, Hafen E, Witters L A, Ellisen L W, Kaelin W G Jr. Regulation of mTOR function in response to hypoxia by REDD1 and the TSC1/TSC2 tumor suppressor complex. Genes Dev. Dec. 1, 2004;18(23):2893-904

Ellisen L W. Growth control under stress: mTOR regulation through the REDD1-TSC pathway. Cell Cycle. 2005 November;4(11):1500-02

Sofer A, Lei K, Johannessen C M, Ellisen L W. Regulation of mTOR and cell growth in response to energy stress by REDD1. Mol Cell Biol. 2005 July;25(14):5834-45.

Zhang Y, Gao X, Saucedo L J, Ru B, Edgar B A, Pan D. Rheb is a direct target of the tuberous sclerosis tumor suppressor proteins. Nat Cell Biol. 2003 June;5(6):578-81.

Tee A R, Manning B D, Roux P P, Cantley L C, Blenis J. Tuberous sclerosis complex gene products, Tuberin and Hamartin, control mTOR signaling by acting as a GTPase-activating protein complex toward Rheb. Curr Biol. Aug. 5, 2003;13(15):1259-68.

Cai S L, Tee A R, Short J D, Bergeron J M, Kim J, Shen J, Guo R, Johnson C L, Kiguchi K, Walker C L. Activity of TSC2 is inhibited by AKT-mediated phosphorylation and membrane partitioning. J Cell Biol. Apr. 24, 2006;173(2):279-89.

Li Y, Inoki K, Vikis H, Guan K L. Measurements of TSC2 GAP Activity Toward Rheb. Methods Enzymol. 2005;407:46-54.

Choi J H, Adames N R, Chan T F, Zeng C, Cooper J A, Zheng X F. TOR signaling regulates microtubule structure and function. Curr Biol. Jul. 13, 2000;10(14):861-4.

Jiang X, Yeung R S. Regulation of microtubule-dependent protein transport by the TISC2/mammalian target of rapamycin pathway. Cancer Res. May 15, 2006;66(10):5258-69.

Gau C L, Kato-Stankiewicz J, Jiang C, Miyamoto S, Guo L, Tamanoi F. Famesyltransferase inhibitors reverse altered growth and distribution of actin filaments in Tsc-deficient cells via inhibition of both rapamycin-sensitive and -insensitive pathways. Mol Cancer Ther. 2005 June;4(6):918-26.

Sarbassov D D, Ali S M, Sengupta S, Sheen J H, Hsu P P, Bagley A F, Markhard AL, Sabatini D M. Prolonged rapamycin treatment inhibits mTORC2 assembly and Akt/PKB. Mol Cell. Apr. 21, 2006;22(2):159-68.

Bulinski J C, Gundersen G G. Stabilization of post-translational modification of microtubules during cellular morphogenesis. Bioessays. 1991 June; 13(6):285-93. Review. Peris L, Thery M, Faure J, Saoudi Y, Lafanechere L, Chilton J K, Gordon-Weeks P, Galjart N, Bornens M, Wordeman L, Wehland J, Andrieux A, Job D. Tubulin tyrosination is a major factor affecting the recruitment of CAP-Gly proteins at microtubule plus ends. J Cell Biol. Sep. 11, 2006;174(6):839-49.

Honore S, Pasquier E, Braguer D. Understanding microtubule dynamics for improved cancer therapy. Cell Mol Life Sci. 2005 December;62(24):3039-56. Review.

Liu H, Derek C. Radisky D C, Nelson C M, Zhang H, Fata J E, Roth R A, Bissell M. Mechanism of Akt1 inhibition of breast cancer cell invasion reveals a protumorigenic role for TSC2. Proc Natl Acad Sci U S A. Mar 14, 2006;103(11):4134-9.

Birukova A A, Birukov K G, Smurova K, Adyshev D, Kaibuchi K, Alieva I, Garcia J G, Verin A D. Novel role of microtubules in thrombin-induced endothelial barrier dysfunction. FASEB J. 2004 December;18(15):1879-90.

1. A process for determining whether a test compound modulates the activity of an RTP801L polypeptide comprising the following steps:
a) providing an RTP801L polypeptide and a second polypeptide selected from the group consisting of RTP801, RTP801L, TSC1, TSC2 and alpha-tubulin;
(b) treating or contacting the polypeptides of a) with the test compound;
(c) determining the amount of a complex comprising the RTP801L polypeptide and the second polypeptide; and
(d) comparing the amount of such complex determined in step c) with the amount determined for control polypeptides not treated or contacted with the test compound.

2. The process of claim 1 wherein a difference in the amount determined in step c) with the amount determined for the control polypeptides indicates that the test compound modulates the activity of RTP801L.

3. The process of claim 1 wherein one or both of the polypeptides are substantially purified.

4. The process of claim 1 wherein the RTP801L polypeptide is a form of RTP801L comprising a tag.

5. The process of claim 1 wherein the second polypeptide is a form of the second polypeptide comprising a tag.

6. The process of claim 1 wherein the RTP801L polypeptide is a form of RTP801L comprising a first tag and the second polypeptide is a form of the second polypeptide comprising a second tag.

7. The process of claim 1 wherein one of the polypeptides is attached to a solid support.

8. A process for determining whether a test compound modulates the activity of an RTP801L polypeptide comprising the following steps:
a) providing a cell which expresses
(i) an RTP801L polypeptide and
(ii) a second polypeptide selected from the group consisting of RTP801, RTP801L, TSCl, TSC2 and alpha-tubulin;
(b) treating or contacting the cell of (a) with the test compound;
(c) determining the amount of a complex comprising the RTP801L polypeptide and the second polypeptide present in the cell; and
(d) comparing the amount of such complex determined in step c) with the amount determined in a control cell not treated or contacted with the test compound.

9. The process of claim 8 wherein a difference in the amount determined in step c) with the amount determined in the control cell indicates that the test compound modulates the activity of RTP801L.

10. The process of claim 8 wherein a lysate is prepared from the cell of step (b) and the detection of step (c) is performed on the lysate.

11. The process of claim 8 wherein a lysate is prepared from the cell of step (a) and the treatment of step b) and detection of step (c) are performed on the lysate.

12. A process for determining whether a test compound modulates the activity of RTP801L comprising the following steps:
a) providing a cell which expresses
(i) a form of RTP801L comprising a first tag; and
(ii) a form of a second polypeptide selected from the group consisting of RTP801, RTP801L, TSC 1, TSC2 and alpha-tubulin, wherein the second polypeptide comprises a second tag;
(b) treating or contacting the cell of (a) with the test compound;
(c) determining the amount of a complex comprising the tagged form of RTP801L and the tagged form of the second polypeptide present in the cell; and
(d) comparing the amount of such complex determined in step c) with the amount determined in a control cell not treated or contacted with the test compound.

13. The process of claim 12 wherein a difference in the amount determined in step c) with the amount determined in the control sample indicates that the test compound modulates the activity of RTP801L.

14. The process of claim 12 wherein a lysate is prepared from the cell of step (b) and the detection of step (c) is performed on the lysate.

15. The process of claim 12 wherein a lysate is prepared from the cell of step (a) and the treatment of step b) and detection of step (c) are performed on the lysate.

16. The process of claim 12 wherein the first tag and the second tag interact to produce a moiety, the amount of which can be determined.

17. A process for determining whether a test compound modulates the activity of an RTP801L polypeptide comprising the following steps:
a) providing an RTP801L polypeptide;
(b) treating or contacting the polypeptide of a) with the test compound;
(c) determining the amount of an RTP801L polypeptide complex; and
(d) comparing the amount of such complex determined in step c) with the amount determined for a control RTP801L polypeptide not treated or contacted with the test compound.

18. The process of claim 17 wherein a difference in the amount determined in step c) with the amount determined for the control polypeptides indicates that the test compound modulates the activity of RTP801L.

19. The process of claim 17 wherein the RTP801L polypeptide is substantially purified.

20. The process of claim 17 wherein a portion of the RTP801L polypeptide is a form of RTP801L comprising a tag.

21. The process of claim 17 wherein a first portion of the RTP801L polypeptide is a form of RTP801L comprising a first tag and the second portion of the RTP801L polypeptide is a form of RTP801L comprising a second tag.

22. The process of claim 17 wherein a portion of the RTP801L polypeptide is attached to a solid support.

23. The process of claim 17 wherein the complex is a dimer.

24. A process for obtaining a compound which modulates apoptosis in a cell comprising:
a) providing cells which express the human RTP801L polypeptide;
b) contacting the cells with a plurality of compounds;
c) determining which of the plurality of compounds modulates apoptosis in the cells; and
d) obtaining the compound determined to modulate apoptosis in step c).

25. The process according to claim 24 comprising:
a) providing cells which express the human RTP801L polypeptide at a level such that about 50% of the cells undergo apoptosis in the presence of a known apoptosis-stimulating agent;
b) contacting the cells with the plurality of compounds;
c) treating the cells with an amount of the known apoptosis-stimulating agent so as to cause apoptosis in the cells;
d) determining which of the plurality of compounds modulates apoptosis in the cells; and
e) obtaining the compound determined to modulate apoptosis in step d).

26. A process for obtaining a compound which modulates the activity of the RTP801L polypeptide comprising:
a) measuring the activity of the RTP801L polypeptide;
b) contacting the RTP801L polypeptide with a plurality of compounds;
c) determining which of the plurality of compounds modulates the activity of the RTP801L polypeptide; and
d) obtaining the compound determined to modulate the activity of the RTP801L polypeptide in step c).

27. A process for obtaining a compound which modulates the activity of the RTP801L polypeptide comprising:
a) measuring the binding of the RTP801L polypeptide to a species with which the RTP801L polypeptide interacts;
b) contacting the RTP801L polypeptide with a plurality of compounds;
c) determining which of the plurality of compounds modulates the binding of the of the RTP801L polypeptide to the species; and
d) obtaining the compound determined to modulate the binding of the RTP801L polypeptide to the species in step c).

28. A kit for obtaining a compound which modulates the biological activity of RTP801L comprising:
(a) RTP801L; and
(b) an interactor with which RTP801L interacts.

29. The kit of claim 28 wherein the interactor is selected from the group consisting of an RTP801 polypeptide, a TSC1 polypeptide, a TSC2 polypeptide and an alpha-tubulin polypeptide.
